SEQUENCE LISTING (371 sequences)

SEQ ID NO: 1 - protein, 32 aa, Cicer arietinum
TKPCQSDKDCKKFACRKPKVPKCINGFCKCVR

SEQ ID NO: 2 - protein, 34 aa, Cicer arietinum
KKMPCKRRRDCKTYPCPHPKVRDCVKGYCKCVVR

SEQ ID NO: 3 - protein, 26 aa, Cicer arietinum
SRDCKTYPCPPSKVKDCIKGYCKCVR

SEQ ID NO: 4 - protein, 33 aa, Cicer arietinum
TKQPCKSRKHCKTYRCPTPKVPNCVNGFCKCVR

SEQ ID NO: 5 - protein, 24 aa, Medicago truncatula
RNGCIVDPRCPYQQCRRPLYCRRR

SEQ ID NO: 6 - protein, 38 aa, Medicago truncatula
EDIGHIKYCGIVDDCYKSKKPLFKIWKCVENVCVLWYK

SEQ ID NO: 7 - protein, 6 aa, synthetic construct
KREAEA

SEQ ID NO: 8 - protein, 34 aa, Cicer arietinum
KMICKTRVDCKKYRCPRSKIKDCVKGYCRCVRKK

SEQ ID NO: 9 - protein, 37 aa, Cicer arietinum
KQVKKSCKSHKDCKTHPCPPSKVKQCVNGYCRCVRIY

SEQ ID NO: 10 - protein, 32 aa, Cicer arietinum
KQRCKSRVDCKTYPCPIPKVKSCLNGYCKCVR

SEQ ID NO: 11 - protein, 50 aa, Cicer arietinum
HPRYPCTKDEDCPKDMCKYPGSYPRCVWGWCECFVKMRPLRRPIKRKQLQ

SEQ ID NO: 12 - protein, 43 aa, Cicer arietinum
YYLTNVTWTKSCPKICNPPNGYCYCHKYREGLPKSLRKXTLND

SEQ ID NO: 13 - protein, 35 aa, Cicer arietinum
KQSCKSWNDCKTYPCPRPNGRNCVNGYCRCYVRIW

SEQ ID NO: 14 - protein, 32 aa, Cicer arietinum
KQDCKSHNDCKTYPCPTSKVKQCVNGYCRCVR

SEQ ID NO: 15 - protein, 54 aa, Oxytropis lambertii
KPVFSHQKKKKKIYIGPDQIPPCRNNKQCAPLICVDRKPWCIKGGCRCVPLGWW

SEQ ID NO: 16 - protein, 49 aa, Oxytropis lambertii
KPPFCRQDKPSFPCRWESDCPKNMCFTGFTPRCIKFVCTCAPSNIRMFF

SEQ ID NO: 17 - protein, 40 aa, Astragalus canadensis
NPFIPIRCIRNSDCPKNMCPLGKTPACTKRRCRCVPQFGA

SEQ ID NO: 18 - protein, 50 aa, Astragalus canadensis
KSVFSHPKKKVYTGPNQYPPCTNDTQCAPLYCLEEGTKSRCMKGRCRCWK

SEQ ID NO: 19 - protein, 50 aa, Astragalus canadensis; identical to SEQ ID NO: 18

SEQ ID NO: 20 - protein, 36 aa, Astragalus canadensis
LRCKQDFDCQQLRCLSGYKPVCHRRRVCKCAPLLSP

SEQ ID NO: 21 - protein, 52 aa, Astragalus canadensis
QRRRRRFPIPLPLGFPSIEWCFTAGDCPVDFCENKVGSKIVCSFLYVCRCRK

SEQ ID NO: 22 - protein, 49 aa, Astragalus canadensis
PPMPKFVPKKPKKAPRCFQDSDCPPDMCLPSLTPKCIMGYCVCVRMMSR

SEQ ID NO: 23 - protein, 31 aa, Astragalus canadensis
SIPCKTDKDCRQVATIPPGIKLRCKNGFCVS

SEQ ID NO: 24 - protein, 38 aa, Astragalus canadensis
ATPCKTDFDCRKNMAPPGYEPRCRKGFCVNVRLYMMWT

SEQ ID NO: 25 - protein, 30 aa, Astragalus canadensis
LPCIRGSNCPRNMCPSGSRTKCIRHVCECR

SEQ ID NO: 26 - protein, 31 aa, Astragalus canadensis
AFPCKRGVDCPKNMCPPWSKPMCERRICKCP

SEQ ID NO: 27 - protein, 37 aa, Astragalus canadensis
SSFPCRQDSDCTKNMCRSGFKPACIRRVCKCMPIFGF

SEQ ID NO: 28 - protein, 35 aa, Astragalus canadensis
LRCKQDSDCQQLRCLSGYKPVCSSWRVCKCARLGP

SEQ ID NO: 29 - protein, 39 aa, Astragalus canadensis
ARPIKRAIPCRYHFHCKNMYCFQEPKCVLGICECKGMYP

SEQ ID NO: 30 - protein, 35 aa, Onobrychis viciifolia
VNTIVVGCIKASDCPKKMCPRSLKRKCIERKCICQ

SEQ ID NO: 31 - protein, 32 aa, Onobrychis viciifolia
GAVILCFKAWNCPKKMCPNGVIPWCMKRKCAC

SEQ ID NO: 32 - protein, 32 aa, Onobrychis viciifolia
EKTGCVTEADCTGCKRKCKVRFIWCLVRGACR

SEQ ID NO: 33 - protein, 37 aa, Onobrychis viciifolia
LFECKTATNCPLAMCTAPHSRWCYKRRCRCIRNEIIM

SEQ ID NO: 34 - protein, 46 aa, Onobrychis viciifolia
KPYPNLVPKAKLGLTIMCFGDQHCPSVCPNGANPRCIKGKCTPACF

SEQ ID NO: 35 - protein, 33 aa, Onobrychis viciifolia
CVVHNDCKHSGGFCTYFRKCVRGECRCARKLYW

SEQ ID NO: 36 - protein, 43 aa, Onobrychis viciifolia
YKSGKPIFPIRGECKMDSHCPGPCPYLHYPSCRNHYCVCLVRR

SEQ ID NO: 37 - protein, 54 aa, Ononis spinosa
SWCRPPKRSRPPQKSIFCKTHADCKKLLCHFPRVRRCIEGMCKCVKISPSGPPV

SEQ ID NO: 38 - protein, 39 aa, Ononis spinosa
SSTHLCSKRRKPWWRSTRTPPKPPPISCVTDFDCKEKRC

SEQ ID NO: 39 - protein, 57 aa, Ononis spinosa
IPPNRRPRIRIFCKSDDDCKEKKCKLPTLSKCVERICKCLKKYIPLVRRYFCQSDAD

SEQ ID NO: 40 - protein, 70 aa, Ononis spinosa
TLFRSMKNYFTFFTRTPHRVSIMRIHSFTIFIIYTILNTILNRCSTFTSCIILNRCCTFTNCTNFSNDEK

SEQ ID NO: 41 - protein, 50 aa, Ononis spinosa
KPKPKPKPKPKPKPKGSICVIHDHCPKEICSEDRTPKCYDGICYCVKTYF

SEQ ID NO: 42 - protein, 32 aa, Ononis spinosa
RSCYENKNCPQKLCPYNLIAACRSHVCKCIKR

SEQ ID NO: 43 - protein, 30 aa, Ononis spinosa
LYCENDGDCKRKFPRWIAKCVNKKCYPLLL

SEQ ID NO: 44 - protein, 44 aa, Ononis spinosa
SAPFKCMTDGDCISKYPLSKLRVVCSRGNCYSLVPIHSTIFSVK

SEQ ID NO: 45 - protein, 46 aa, Ononis spinosa
IPPNNRPRIRIFCKSDIDCKNKTCTTPRIPKCVEGLCKCNLCFVNL

SEQ ID NO: 46 - protein, 32 aa, Ononis spinosa
TPIPCIRDGDCPRRVCIFPFKVRCVRTNCKCF

SEQ ID NO: 47 - protein, 43 aa, Ononis spinosa
RGCYSVNDCMERLPFPCERGRSDQLVKCQNMQCVCIQKKKKFP

SEQ ID NO: 48 - protein, 34 aa, Ononis spinosa
NVKCVKDSDCRKVMCPVGSRPRCYFNYCKCPMVF

SEQ ID NO: 49 - protein, 31 aa, Ononis spinosa
LTPYCRDSRNCPPFFCKYPKVAKCIQFKCTC

SEQ ID NO: 50 - protein, 36 aa, Ononis spinosa
RRFTKCTNNKECKGYYRCRSRESPVCFMGLCDCMLT

SEQ ID NO: 51 - protein, 44 aa, Ononis spinosa
KPKPKPKRKGSNCVIHDHCPKEICSDDRTPKCYDGICYCVKTYF

SEQ ID NO: 52 - protein, 44 aa, Galega orientalis
WFKPIPCVSKKDCPPIKSTTFIHRCIGGLCKVRKRGIKGFINIP

SEQ ID NO: 53 - protein, 37 aa, Galega orientalis
QLTRLSNCKTDVDCPVSNRVVLRCRKGWCRAGFARKY

SEQ ID NO: 54 - protein, 30 aa, Galega orientalis
IITNRRCKSHKDCPGFGKCLKLKCVYTPKR

SEQ ID NO: 55 - protein, 39 aa, Galega orientalis
ANLLVPCISDADCPKTQKAIFRCRKGYCRSFPKFGRLKK

SEQ ID NO: 56 - protein, 39 aa, Galega orientalis; identical to SEQ ID NO: 55

SEQ ID NO: 57 - protein, 37 aa, Galega orientalis
GGPRHCNRDADCQQFRSMIRYIFKCINKRCKKVPNLI

SEQ ID NO: 58 - protein, 66 aa, Galega orientalis
QILLRKTFFIILKLSSLRYSLHMKFYHTLVIMFFIAAYKRCETDADCRLSRKHKFVCRKGWCEPHL

SEQ ID NO: 59 - protein, 36 aa, Galega orientalis
NGSLQRFCKSNKDCPSSHRNIKVRCVKGMCVAYTKI

SEQ ID NO: 60 - protein, 31 aa, Galega orientalis
YGIKGCKTNKDCPTGSKCKKGRCVHYRPSFK

SEQ ID NO: 61 - protein, 33 aa, Galega orientalis
FVNIPCKKNKDCPEPVFGYFKCRKGFCKRFHKS

SEQ ID NO: 62 - protein, 46 aa, Galega orientalis
NFYHVLLTMVFIAVQRFCKSNKDCPSSHRNIKVRCVKGMCVAYTKI

SEQ ID NO: 63 - protein, 52 aa, Galega orientalis
YGKLYSNPSQIFIASNNILIPCKTVKNCPPPLRNGSYRCSKGYCVLIQRYRP

SEQ ID NO: 64 - protein, 39 aa, Galega orientalis
APFLRNSQIRCKTDKDCPFSISIRRHYWCRNGYCKPMNN

SEQ ID NO: 65 - protein, 39 aa, Galega orientalis
SFYYQPCKTDGDCPKSKSFVYRCRKSYCQPYSRFQPKRV

SEQ ID NO: 66 - protein, 45 aa, Galega orientalis
SFYNQPCKTDGDCPKSKSLVYRCRKGYCLPYSRFQSKRVQKYPTE

SEQ ID NO: 67 - protein, 49 aa, Galega orientalis
QYPCVTSDDCPKEITSRFYYRCINNKCRVYDKVVKKFTKIRGYKNVIRE

SEQ ID NO: 68 - protein, 41 aa, Galega orientalis
NIRLISCKSYKDCPISPFDDFKCRKGYCKPFPKPSKNELLR

SEQ ID NO: 69 - protein, 30 aa, Galega orientalis
QFNGFLPCETNKNCPRHSKCRKGFCQHPFR

SEQ ID NO: 70 - protein, 31 aa, Galega orientalis
GNIPCKTNKDCPIPLFGYLKCVKGYCRRYLR

SEQ ID NO: 71 - protein, 37 aa, Galega orientalis
QFPCETHADCPYDIILYRYRCIHGICNLVRKKRKRNK

SEQ ID NO: 72 - protein, 55 aa, Galega orientalis
KNIDHKQIILSRFNPTSNIIPCKTDADCPKLHKDYAFSYQCINKKCVFRSKVMMN

SEQ ID NO: 73 - protein, 36 aa, Galega orientalis
QFNGRGPCKTDKDCYIIFKCRKGYCERRHNVYVLKP

SEQ ID NO: 74 - protein, 44 aa, Galega orientalis
LDIPCKTGADCPKPTSAYRMYKCIDFKCFELYRPRNPRPRKPQF

SEQ ID NO: 75 - protein, 38 aa, Galega orientalis
VSFTRPRKCGPCGPKLPDSSCPPPKKWKCIPPGVWVCS

SEQ ID NO: 76 - protein, 41 aa, Galega orientalis
NFYHVLVTKLFIAINLPCKTNGDCPTTSKCKKGQCIKRAYY

SEQ ID NO: 77 - protein, 46 aa, Galega orientalis
GRIKENGYPCVTNADCPQAHRLTYKSLCINNKCRYYRSRIGNEFIK

SEQ ID NO: 78 - protein, 31 aa, Galega orientalis
NRPCTSDADCPISRRTIFRCRGGWCSPYPKR

SEQ ID NO: 79 - protein, 43 aa, Galega orientalis
FTCKTRADCPKYHKPQFYYECIKRNCELYSTTNKPWIPQKRLQ

SEQ ID NO: 80 - protein, 46 aa, Galega orientalis
FPCQIDADCPKSANPSLLYKCLNFTCFLCCGPLKPFKPRKPRKPRS

SEQ ID NO: 81 - protein, 40 aa, Galega orientalis
TMINIPYTSTPCKTSADCPKRIMDIVFKCRKGWCEGFRTR

SEQ ID NO: 82 - protein, 69 aa, Galega orientalis
VIIKDNGYPCVTNADCPPSGRITYKSLCHNNQCRYYRSRIGKEFIEYQTQEIGNVMPRRMSSYVRNKNM

SEQ ID NO: 83 - protein, 69 aa, Galega orientalis; identical to SEQ ID NO: 82

SEQ ID NO: 84 - protein, 60 aa, Galega orientalis
SIECYADSDCLKTMCDQRPWNQWCINNKCVCVHTYRTKNPSSKRPKRVHKSLARKNEPLH

SEQ ID NO: 85 - protein, 35 aa, Galega orientalis
YGIITCKTNKDCPTGSKCKKGVCKLNNMRHFNHEY

SEQ ID NO: 86 - protein, 34 aa, Galega orientalis
HFMIPCKSDGDCPRHANGFYRCRKGYCQPYRSRP

SEQ ID NO: 87 - protein, 56 aa, Galega orientalis
ECITHVDCYKIYPCPNGALLKCLNKVCYQNTPPWEKREVFQRKGRPMTKAHMPRNL

SEQ ID NO: 88 - protein, 32 aa, Galega orientalis
IIPCKTVDDCPKTKSLVYICRKGYCYARFAKT

SEQ ID NO: 89 - protein, 39 aa, Galega orientalis
YNSRFKLCKTDADCPKIPDSSETYYGCRKGICRHFFKLK

SEQ ID NO: 90 - protein, 42 aa, Galega orientalis
KIVLHRSCKTDADCPVTTQLNFIYRCLNFYCFKCYKHRKPWY

SEQ ID NO: 91 - protein, 42 aa, Galega orientalis
IRSSKASNYNLCNTDADCPKFSDSSEFYISCRKFVCRKFYKK

SEQ ID NO: 92 - protein, 39 aa, Galega orientalis
YYSNFKLCKTDADCPKPPNSPETYYGCRKGICRHFFKLK

SEQ ID NO: 93 - protein, 43 aa, Galega orientalis
FICETTNDCPKYRKPQFYYVCIKRKCELYSATDKPWIPQKRLQ

SEQ ID NO: 94 - protein, 74 aa, Galega orientalis
RNTYFYHSKMSLFNSYSLYTIFYQVLLIIFFIVESIDIPCKTDVDCPKSTRSSLIYRCFNFKCFLCCRPRKPRY

SEQ ID NO: 95 - protein, 49 aa, Galega orientalis
FPCKRSIDCPRYHKPQYHYLCIKQKCELFLTVYEPSRTDNPWIPYKRVQ

SEQ ID NO: 96 - protein, 33 aa, Galega orientalis
MPMYPKPCKTKRQCPSCFHPHKVDCVKGFCRCN

SEQ ID NO: 97 - protein, 46 aa, Galega orientalis
SNKDPITCHHHKDCANHKCTIRHRVPKCLFYWCACVRKSLVIKLDY

SEQ ID NO: 98 - protein, 37 aa, Galega orientalis
PLIPRLSCITDVDCQTSDKLKFRCRKGLCKPYFKHCP

SEQ ID NO: 99 - protein, 33 aa, Galega orientalis
KLDYKRCKTNTDCEKPCKCFRGRCKTVFSDLNP

SEQ ID NO: 100 - protein, 37 aa, Galega orientalis
EYTFFSICKIDADCPLHPRLIVRCSNGRCKFSGVLRR

SEQ ID NO: 101 - protein, 34 aa, Galega orientalis
QILLPYKRCETDADCRLSRKHKFVCRKGWCEPHL

SEQ ID NO: 102 - protein, 37 aa, Galega orientalis
WFTDPIPCVSKRDCPPIKSPDFVYKCIGGFCKINPRK

SEQ ID NO: 103 - protein, 44 aa, Galega orientalis
IDIPCRTDADCPKSTSPSLIYKCIDFKCFELFSPRTPRPRKPRF

SEQ ID NO: 104 - protein, 35 aa, Galega orientalis
QFTCETIADCPPPREKNNKIICINNICRYYRSRMK

SEQ ID NO: 105 - protein, 34 aa, Galega orientalis
VVLYLCKTDADCRKKLQSFVHWCVKGYCQPSSRF

SEQ ID NO: 106 - protein, 40 aa, Galega orientalis
QVPCRTVEDCPKHMDSRFAYVCYRKKCRLLFKNIQGSSEQ

SEQ ID NO: 107 - protein, 39 aa, Galega orientalis
IDVIPCKKDVDCPENWYFFPYIHKCISKKCKWIKAPGRF

SEQ ID NO: 108 - protein, 37 aa, Galega orientalis
TKRCITDDDCPKFFSTVPYEYKCVNKKCIKLPKITTT

SEQ ID NO: 109 - protein, 37 aa, Galega orientalis
GYHEVMVCKTNKDCPSMSTRYSYFKCYKGYCKAFCKY

SEQ ID NO: 110 - protein, 39 aa, Galega orientalis
SGGRLIEGFPIPCKTDKDCPFNPNTHRYRCRKGYCTFIY

SEQ ID NO: 111 - protein, 39 aa, Galega orientalis
VNVVFSRFCKIDRDCYAYQRCRNGVCTPPKPGDYHIHKP

SEQ ID NO: 112 - protein, 43 aa, Galega orientalis
NCIMGCRDDSDCSNSICILPKYPLCRKSLCRCVKKKNQLRSFV

SEQ ID NO: 113 - protein, 37 aa, Galega orientalis
ERKYSYCNTYTDCPPSTRVAYVIKCIHHKCIYFSKGL

SEQ ID NO: 114 - protein, 28 aa, Galega orientalis
WPCKHKRDCTICYPPLKPDCIKRKCHCV

SEQ ID NO: 115 - protein, 48 aa, Galega orientalis
MIIFSLLFIVVMDRFLNRLPWECRNKRDCPTCYHPKKRDCIKHFCGCF

SEQ ID NO: 116 - protein, 32 aa, Galega orientalis
YCRMNSECPKKMCTPFMIRKCIWNRCECVTLR

SEQ ID NO: 117 - protein, 38 aa, Galega orientalis
ENIRKCKSYLDCMDFCILPQYRRCIWDKCICTKGKQIR

SEQ ID NO: 118 - protein, 40 aa, Galega orientalis
KRCSFTWDCYYLCPVGKVAVCNWGRCTCANSYFPRVINRI

SEQ ID NO: 119 - protein, 40 aa, Galega orientalis
GSIPCKKYTDCREMLCLPLQVRQCKNSYCQCVKKYFYIRE

SEQ ID NO: 120 - protein, 40 aa, Galega orientalis
TMINIPYTSTPCKTSADCPKRMMDIVFKCCKGWCEKFRIF

SEQ ID NO: 121 - protein, 27 aa, Galega orientalis
GCPKDHCRYPKIPYCNLFWRCRCGNIL

SEQ ID NO: 122 - protein, 42 aa, Galega orientalis
YSRLTKEKLYSCFTNTDCPPPEDYHKCLCHNNICRYYRSRMK

SEQ ID NO: 123 - protein, 34 aa, Galega orientalis
GFHACRSDADCRINQCPPLLVPKCIVCKCRCRKV

SEQ ID NO: 124 - protein, 76 aa, Galega orientalis
ECITDVDCYKIYLCPYGSYIKCLNKMCYEITPETVHIYNKSGHQRSISKERKTNDKGAHAKKPMKVLNEMVKTLME

SEQ ID NO: 125 - protein, 36 aa, Pisum sativum
AAVAIGCVTDNDCPSRTASRRYKCVRNKCRHTRVMH

SEQ ID NO: 126 - protein, 36 aa, Pisum sativum
PSGLRCLNDSDCLRFRCSKIYKVLCIERRCRRIKMH

SEQ ID NO: 127 - protein, 36 aa, Pisum sativum
AVATKCKTDVDCPPSTSIRRYKCTKNKCRYTRLSYG

SEQ ID NO: 128 - protein, 50 aa, Pisum sativum
YNLKYCTNDKDCPTMMCFPPDVSKCVWKTCYCVQKHKKKLKKKKKLTFNM

SEQ ID NO: 129 - protein, 35 aa, Pisum sativum
YKCSKDSDCFVLRCSKHFRPVCVNRRCVRVRILID

SEQ ID NO: 130 - protein, 43 aa, Pisum sativum
RIAKCVRDGDCFTHLCQPPRMVKCDNKKCTCVRVTSILRYVSK

SEQ ID NO: 131 - protein, 38 aa, Pisum sativum
LRKKCRTDSDCPKQMCRFPTYNKCVIRNHCQCVLSKII

SEQ ID NO: 132 - protein, 39 aa, Pisum sativum
PAGNMGCIRDSDCSFLFCRKGYKAVCINKRCKRVKIMVD

SEQ ID NO: 133 - protein, 37 aa, Pisum sativum
LARMRCLKDSDCSFYVCRKSYKAIFTGTRCRCVKIID

SEQ ID NO: 134 - protein, 71 aa, Pisum sativum
GSRNLFSHHFQIFFYKLVSFIYHVSNNIILLCLFFTARRFIPCKTRADCPDSSEFQKYMCIESLCKLIRLK

SEQ ID NO: 135 - protein, 34 aa, Pisum sativum
FRHQCLEHSHCYRVLCRRPAIPKCDFRRGLCKCV

SEQ ID NO: 136 - protein, 42 aa, Pisum sativum
FYQRCSSDSDCRLNKCERPRISKCVRNWCRCLHPKELISRNQ

SEQ ID NO: 137 - protein, 42 aa, Pisum sativum; identical to SEQ ID NO: 136

SEQ ID NO: 138 - protein, 41 aa, Pisum sativum
WMRRCFKDSDCPKDWCRKPKIMTCVSHSLCKCMVQGYLKPI

SEQ ID NO: 139 - protein, 42 aa, Pisum sativum
WYGRRCSRDSDCRINMLCRKPKIAKCVARMCNCVVSTYSIPE

SEQ ID NO: 140 - protein, 42 aa, Pisum sativum
RQACVHDRECKCPGHPVSRCVKGFCRCYTFPAIPSFKGMNKY

SEQ ID NO: 141 - protein, 40 aa, Pisum sativum
WKRTCFKDSHCPDSCRRPKIMKCVFQSYCKCVMPGYIAPP

SEQ ID NO: 142 - protein, 43 aa, Pisum sativum
TFYRCFDDKDCVKRLYCRIPTKAKCVSIFCKCRIDKELLNPRA

SEQ ID NO: 143 - protein, 42 aa, Pisum sativum
GKSIKCSKDLDCPKSMCKSPMRVKCWYSSFCRCYNAATYNPW

SEQ ID NO: 144 - protein, 40 aa, Pisum sativum
VRDKPHPYFSSPCQTKTDCRDKACYRPLKRKCISYRCECA

SEQ ID NO: 145 - protein, 38 aa, Pisum sativum
GIIPEKTHKKCEKDIHCPRSSCPPYLYAKCVRFRCYCR

SEQ ID NO: 146 - protein, 50 aa, Pisum sativum
INNDNKSFFTKLSPYANYPISCIRTKDCPKSWCYPPQKPKCIRSKCDCLH

SEQ ID NO: 147 - protein, 38 aa, Pisum sativum
PKHITCLQDSDCSILFCWKSYKAICVNKRCKRVKLLID

SEQ ID NO: 148 - protein, 36 aa, Pisum sativum
AKNKCSEDSDCWYLYCSRTFKAICVDRKCKRVKILG

SEQ ID NO: 149 - protein, 33 aa, Pisum sativum
AAIKCEVQADCPKIPNLFPKFFKCINKKCILFT

SEQ ID NO: 150 - protein, 41 aa, Pisum sativum
ATPAAIDCITDADCPNRTQIFRYKCVNKKCKGTRVTSITLL

SEQ ID NO: 151 - protein, 35 aa, Pisum sativum
QIKCKTNDDCPKIPHLFPSIYRCIGNKCIIGTVKS

SEQ ID NO: 152 - protein, 40 aa, Pisum sativum
KNDNNCYRFNYLCMPPKIMRCYNKQCTCVRLTSLFHYVPV

SEQ ID NO: 153 - protein, 37 aa, Pisum sativum
LKHMSCVTHSDCSNLLGAKSYMVICKNKICRYVSLAL

SEQ ID NO: 154 - protein, 37 aa, Pisum sativum
AKVKCKKNGDCPKLPHMFPIIYRCYQQECTLVRVLDS

SEQ ID NO: 155 - protein, 43 aa, Pisum sativum
NIKCKINGDCPKILHLYPYIYRCFNQRCRLVREMPVSDSRDFT

SEQ ID NO: 156 - protein, 37 aa, Pisum sativum
KNNQASFRCKTDADCLKVLPLMWKCIYYECTYVKKLQ

SEQ ID NO: 157 - protein, 41 aa, Pisum sativum
WHGSRRCLTDSDCNKNKCRKPTIIKCVDRLCTCVAVFQPQS

SEQ ID NO: 158 - protein, 44 aa, Pisum sativum
ERIKCFGDSDCKNRMFCRSPFKVTCINNNHCKCVLARELLNPRI

SEQ ID NO: 159 - protein, 40 aa, Pisum sativum
IEATRFKCTQDADCLCLPCWKSFKAVCIRKRCVRVKVMID

SEQ ID NO: 160 - protein, 39 aa, Pisum sativum
FIRCVTVDDCSRRVCGFGLKRICYRDFCRCVDPATRIKP

SEQ ID NO: 161 - protein, 38 aa, Pisum sativum
SINRCFRDSDCPPNLCQGPYVAKCVGRRCRCTKVITPI

SEQ ID NO: 162 - protein, 48 aa, Pisum sativum
AEGRPTIFQRFPCQTDVDCRNRACHRPLKRKCVFFYCECVEVLKIFKI

SEQ ID NO: 163 - protein, 43 aa, Pisum sativum
RISRCRNDSDCFNRACQPPRIVKCDNKQCACLKISSLFQYVPL

SEQ ID NO: 164 - protein, 33 aa, Pisum sativum
VPIKCSGDHDCPKNWCPLSMKHKCKSGYCKCVK

SEQ ID NO: 165 - protein, 37 aa, Pisum sativum
NNKIFCYKVEHCDSSICRSPYVLKCRMFRCICERTKQ

SEQ ID NO: 166 - protein, 42 aa, Pisum sativum
TYKCNDDHDCPKKWCPFSMKHKCKGGYCKCVKMTVLRDSVPT

SEQ ID NO: 167 - protein, 44 aa, Pisum sativum
EKIKCFDDSDCENKIYCRPPYRGKCMNRTNCKCIIVRELLNPRR

SEQ ID NO: 168 - protein, 32 aa, Pisum sativum
RIPCHNNHGCTRYLSCPPPLVAKCYNFYCKCR

SEQ ID NO: 169 - protein, 40 aa, Pisum sativum
QACIHDRQCRCTQHTVSKCINGFCKCYTYSDIRLKLMNKY

SEQ ID NO: 170 - protein, 42 aa, Pisum sativum
VYLHFCYEDKDCLQMRCIVPNSPTCVGSVCRCVYQKRKKVLK

SEQ ID NO: 171 - protein, 42 aa, Pisum sativum
RRQNCKEHSDCANYFCVTPYVPKCLYLKCRCRLLDGKLMLRD

SEQ ID NO: 172 - protein, 35 aa, Pisum sativum
YTLCKKRYDCPRDMCIPRITPKCMNGICMCIYTKN

SEQ ID NO: 173 - protein, 37 aa, Pisum sativum
KLIKFPILKCKTVADCPKFITKWVAIMCVDQCCAIVY

SEQ ID NO: 174 - protein, 43 aa, Medicago sativa
AFVPNSGPCKTDKDCKQVKGYMARCRKGYCMQSVKRIRSSYSR

SEQ ID NO: 175 - protein, 40 aa, Medicago sativa
TPQKKVTDVSRPCRTDKDCPKVEFGYKLRCRKGWCVHIPR

SEQ ID NO: 176 - protein, 42 aa, Medicago sativa
IIVCDFAFLPTSRNCITDKDCRQVRNYIARCRKGICQQRPRR

SEQ ID NO: 177 - protein, 34 aa, Medicago sativa
SINLRNHSCIKDKDCPRVGILNIRCRKGFCVHIS

SEQ ID NO: 178 - protein, 32 aa, Medicago sativa
IECRTVADCPKLISSKFVIKCIKKRCVAQFFK

SEQ ID NO: 179 - protein, 35 aa, Medicago sativa
FPDKIFFGDCQTDKDCKPKRGVNFRCRKGKCFPVR

SEQ ID NO: 180 - protein, 38 aa, Medicago sativa
TPQKKVTDVSRPCRMDKDCPKVQYGYKLRCRKGQCVHM

SEQ ID NO: 181 - protein, 36 aa, Medicago sativa
QKTKTPCKNVNDCPKAIKPIFVKCLGNFCHYSIGKI

SEQ ID NO: 182 - protein, 63 aa, Medicago sativa
YLKCKTVDDCPKSQVVYKCVANHCRSVRIRRWILDCKVGRRNPGSNPGPHRCVSLISVDHHFI

SEQ ID NO: 183 - protein, 61 aa, Medicago sativa
ESNRTQRNDITKNYCKKNADCPKHMCIRPQKPKGIDSWCKCIGIDSSISARRDRIIQSSTK

SEQ ID NO: 184 - protein, 36 aa, Medicago sativa
IHFHVSRPCMKDNDCAPVKYYNIRCRKGFCVQIRKY

SEQ ID NO: 185 - protein, 36 aa, Medicago sativa
TYLPTTRPCITDKDCPNVKNYIGRCRKGYCQASKLR

SEQ ID NO: 186 - protein, 39 aa, Medicago sativa
IPYYNFGRCVTDKDCPKVSGDNIRCRKGECVRIPYNRKK

SEQ ID NO: 187 - protein, 55 aa, Medicago sativa
LKFVYVLILFLSIFLIMIVYDSKSFYFSRRCQTDKDCPKNPPHNIRCRKGFCVQI

SEQ ID NO: 188 - protein, 33 aa, Medicago sativa
FQSSVNRPCLSDKDCKKIHGINNRCRKGFCTPI

SEQ ID NO: 189 - protein, 36 aa, Medicago sativa
YHLCKTRFDCPRTYLLFFPRMWKCINRRCRYVYFFE

SEQ ID NO: 190 - protein, 49 aa, Medicago sativa
KSLNGGGKDKCFRDSDCPKHMCPSNMVAKCIHRLCKCRRPELQVQLNPK

SEQ ID NO: 191 - protein, 48 aa, Medicago sativa
YPPCETVADCPKSWFRIYRCEDNFCRYSEKIRRLKPPLRKKIQNKMDN

SEQ ID NO: 192 - protein, 45 aa, Medicago sativa
NFECKSTNDCPKSVLRVWRCINNYCRPVRLKKIHKHHVGENEFLH

SEQ ID NO: 193 - protein, 29 aa, Medicago sativa
FPGCITDKYCQKCRGVNFRCLKGNCVRLR

SEQ ID NO: 194 - protein, 26 aa, Medicago sativa
QTYGHCPRLMCGPKTTKCKKNVCKCV

SEQ ID NO: 195 - protein, 32 aa, Medicago sativa
FPLMPQPCMTHKDCPKIHNIGVKCRKGYCVRI

SEQ ID NO: 196 - protein, 57 aa, Medicago sativa
ESNRTQRNDITKNYCKKNADCPKHMCIRPQKPKGIDSWCKCIGIEVFSSIKGVSARC

SEQ ID NO: 197 - protein, 57 aa, Medicago sativa
FKTYKECKSASDCYTIYRRAFYGSMRCVKGFCKHLRDVKLTVGYATCMYGIARLLDV

SEQ ID NO: 198 - protein, 46 aa, Medicago sativa
WRIPTRPNCKENYGCPIFFCATWIPKCIKNKCYCIRPWGVNSAVKY

SEQ ID NO: 199 - protein, 64 aa, Medicago sativa
VYIKCKTDADCPKSESTIFAMKCNNGQCIYDYIHKRNSYATYLVFLPKKKKNLLLVIVFFIFLF

SEQ ID NO: 200 - protein, 33 aa, Medicago sativa
VLIDCKHVRDCPKGSWSRMYKCIDNKCRFTYPR

SEQ ID NO: 201 - protein, 34 aa, Medicago sativa
ESIECRTVADCPKLISSKFVIKCIKKRCVAQFFK

SEQ ID NO: 202 - protein, 39 aa, Medicago sativa
DGVKNTNRECIQTSDCYKKYQFIPWGKVRCVKGRCRLEM

SEQ ID NO: 203 - protein, 71 aa, Medicago sativa
GQISIQNKRRMNMTQTLIFIYALFIFVSLFLVVTSETRIPCVSRNDCPKRPYPLFMKCVDNFCEIWKIGKE

SEQ ID NO: 204 - protein, 24 aa, Medicago sativa
RNGCIVDARCPYQKCSYPLYCRRR

SEQ ID NO: 205 - protein, 35 aa, Medicago sativa
KQSRIPCTSNRDCPRRLYPKYVVCIEGSCRFHYPL

SEQ ID NO: 206 - protein, 74 aa, Medicago sativa
KQFFNLPKFPSLLCKQYFILFSYILLFYFFITAGVRCHDVSSCPKKMYCKVGTHMQCVKHQCKCIKNFEPKDPA

SEQ ID NO: 207 - protein, 30 aa, Medicago sativa
QVKCKTVKDCPIRRNRKYYCLFGECKYDVI

SEQ ID NO: 208 - protein, 39 aa, Medicago sativa
LKSNRECILTDDCYKRYPNAPFGKLACIKGYCKVLNSLR

SEQ ID NO: 209 - protein, 46 aa, Medicago sativa
EFHRLKFCGTDEDCRRSGSNQHWVYKCINYKCQYVPSKKKCQYVHR

SEQ ID NO: 210 - protein, 51 aa, Medicago sativa
LILFISTFLVIRVYDFIPYVNYGPCVTHKDCPKLPQYHISCRRGQCIRKLL

SEQ ID NO: 211 - protein, 33 aa, Medicago sativa
KWPGCKAAIDCGKNFCIRPLKAKCIMFTCFCVR

SEQ ID NO: 212 - protein, 37 aa, Medicago sativa
AHKRCRVDFDCRMRMCVYPTVPVCIDRLCRCRRPPTM

SEQ ID NO: 213 - protein, 36 aa, Medicago sativa
HKRCRVDFDCRMRMCVYPTVPVCIDRLCRCRRPPTM

SEQ ID NO: 214 - protein, 35 aa, Medicago sativa
LRLRCVHVSKCPKMYCNRGYELSCVQKYCQCVRIT

SEQ ID NO: 215 - protein, 51 aa, Medicago sativa
ESNRTQRNDITKNYCKKNADCPKHMCIRPQKPKGIDSWCKCIGVSLSGHVD

SEQ ID NO: 216 - protein, 59 aa, Medicago sativa
SYIIFISSHFYDISLLYSFTIGYVACITSNDCPPNTRLIRCKCINGKCRFYRRIVLQIV

SEQ ID NO: 217 - protein, 32 aa, Medicago sativa
HTPPPCITDKDCPNIRKTIRCRKGFCVEIIKM

SEQ ID NO: 218 - protein, 62 aa, Medicago sativa
YLKCKTVDDCPKSQVVYKCVGNHCHAVRIRRWGLESLAQLTNVKIVRPDVATCVQTRDLTAV

SEQ ID NO: 219 - protein, 31 aa, Medicago sativa
APKPCVTPKDCPKTFFPIGYKCIKNLCILVI

SEQ ID NO: 220 - protein, 38 aa, Medicago sativa
SNYSPTPFPCMLDIDCTRRKGFSVTCRKGFCVEFKHFK

SEQ ID NO: 221 - protein, 42 aa, Medicago sativa
KTFLMAEYIKCDTDADCPKILHRSFYKCIDNLCKRFRRQHIV

SEQ ID NO: 222 - protein, 41 aa, Medicago sativa
YDSIYFRKSPPCITDKDCPNLKDFTVRCRKGFCIQIHKLTP

SEQ ID NO: 223 - protein, 51 aa, Medicago sativa
MTLLFSIILVLIVYDGIYFRKPPSCIRDKDCPQVKNSNVRCRKGFCIEIFS

SEQ ID NO: 224 - protein, 38 aa, Medicago sativa
YDSIPYVNSGPCVTDKDCPKVSQYNIRCRKGQCVRIRV

SEQ ID NO: 225 - protein, 37 aa, Medicago sativa
LKKCITDEDCPISKTRVYKCLHGECRYTIPYIPKNKF

SEQ ID NO: 226 - protein, 42 aa, Medicago sativa
KTFLMAEYIKCDSDADCPKILHRSYYKCIDNLCKRFRRQHIV

SEQ ID NO: 227 - protein, 37 aa, Medicago sativa
YRNIKYCFIDTDCPRSMSRYPEIVRCVVQCKCVRMMP

SEQ ID NO: 228 - protein, 40 aa, Medicago sativa
VRCHDVSSCPKKMYCKVGTHMQCVKHQCKCIKNFEPKDPA

SEQ ID NO: 229 - protein, 39 aa, Medicago sativa
KFTRCFRDSDCPKTLCHSPGKAKCMHHSICKCIFFGYNI

SEQ ID NO: 230 - protein, 35 aa, Medicago sativa
HVTCKKNADCPKYLCMLPLKPKCNETFWCTCIGKK

SEQ ID NO: 231 - protein, 45 aa, Medicago sativa
APGRKPFNPMNCQTIVDCRYKACYRPLKPQCIDGKCVCMPVLQLS

SEQ ID NO: 232 - protein, 44 aa, Medicago sativa
AIRIRRLNCNLDDGCPNNYCTPPHVGKCVSKHCVCMKPAWYTKS

SEQ ID NO: 233 - protein, 38 aa, Medicago sativa
SNTLLAFRECVSDKDCPIMPRCNIRCRKGLCIPVGKTF

SEQ ID NO: 234 - protein, 42 aa, Medicago sativa
LHCNDANECPPSTWKPFVRCKMNRCIYSRVEPPFGCRVRAQT

SEQ ID NO: 235 - protein, 36 aa, Medicago sativa
ERLYRCLIILIVPTFMCSPGFNPKCMNPRVCKCVHV

SEQ ID NO: 236 - protein, 41 aa, Medicago sativa
KTINISLIRHSPNIFVKQFRCHVSNCSFSCGHCYNAILCWI

SEQ ID NO: 237 - protein, 29 aa, Medicago sativa
SSFTRCRNDVECPQYSCLRGLKMKCICFK

SEQ ID NO: 238 - protein, 96 aa, Medicago truncatula
MQIGKNMVETPKLDYVIIFFFLYFFFRQMIILRLNTTFRPLNFKMLRFWGQNRNIMKHRGQKVHFSLILSDCKTNKDCPKLRRANVRCRKSYCVPI

SEQ ID NO: 239 - protein, 36 aa, Medicago truncatula
AFIQLSKPCISDKECSIVKNYRARCRKGYCVRRRIR

SEQ ID NO: 240 - protein, 35 aa, Medicago truncatula
FLPTSRNCITNKDCRQVRNYIARCRKGQCLQSPVR

SEQ ID NO: 241 - protein, 36 aa, Medicago truncatula
AFLPRSRTCITDKDCLQVRNYIARCRKGICQQHPRR

SEQ ID NO: 242 - protein, 60 aa, Medicago truncatula
ESNWSQRNDITKIHCIKNADCPKHMCIRPQKPKCFDSWYMSQNQNQGNVRVMRRTKKNRL

SEQ ID NO: 243 - protein, 42 aa, Medicago truncatula
ITISNSSFGRIVYWNCKTDKDCKQHRGFNFRCRSGNCIPIRR

SEQ ID NO: 244 - protein, 36 aa, Medicago truncatula
AFIQLSKPCITDKDCSPMKNYKARCRKGYCVSRRIR

SEQ ID NO: 245 - protein, 47 aa, Medicago truncatula
RRVSYNSFFRALPVCQTAADCPEGTRGRTYKCINNKCRYPKLLKPIQ

SEQ ID NO: 246 - protein, 47 aa, Medicago truncatula
LNGHGRNRCFRDSDCPKVMCPSYLVTKCFKKHCRCRKPGLQVQLNPK

SEQ ID NO: 247 - protein, 37 aa, Medicago truncatula
FGMTLRPCLTDKDCPRMPPHNIKCRKGHCVPIGKPFK

SEQ ID NO: 248 - protein, 34 aa, Medicago truncatula
ITPWLKKTCVTDKDCPKVAKINIRCRRGQCVQVF

SEQ ID NO: 249 - protein, 34 aa, Medicago truncatula
ITPWLKKNCVTDKDCPKVAKINIRCRRGQCVQVF

SEQ ID NO: 250 - protein, 38 aa, Medicago truncatula
NSFNSKIVFTDCKTDKDCQNHRGFNFRCRKGNCVAKIR

SEQ ID NO: 251 - protein, 34 aa, Medicago truncatula
ITPWLKKICVTDKDCPKVAKINIRCRRGQCVQVF

SEQ ID NO: 252 - protein, 110 aa, Medicago truncatula
TNFKRKQIPFYFFIREFYPCFIDGNCPRNMCKVYQIPKCVGGLCRCIPLRQVRWKLRLQNSLSPKQKFDDFDKNGIRQGCIFDNTRFKFSEEARLNVHTRGKDAKDVERI

SEQ ID NO: 253 - protein, 34 aa, Medicago truncatula
FPDKIFIGDCKTDKDCKPKRGVNFRCRKGKCYPR

SEQ ID NO: 254 - protein, 36 aa, Medicago truncatula
IYFPVSRPCITDKDCPNMKHYKAKCRKGFCISSRVR

SEQ ID NO: 255 - protein, 24 aa, Medicago truncatula
RNGCIVDPRCPYQQCRRPLYCRRR

SEQ ID NO: 256 - protein, 31 aa, Medicago truncatula
MKNGCKHTGHCPRKMCGAKTTKCRNNKCQCV

SEQ ID NO: 257 - protein, 63 aa, Medicago truncatula
MLIGKKMVETLKLIYVIHLFLSIFLFTNSPFGQIIFRQCKTDKDCPKLGRANIRCREGYCVRI

SEQ ID NO: 258 - protein, 47 aa, Medicago truncatula
ISCDVKVLAIVIQHLCLLLKGYTSALKDIVMRCVKGYCKHLKVVKIR

SEQ ID NO: 259 - protein, 35 aa, Medicago truncatula
YLKCKTVHDCPKSQVVYRCVGNYCRAVKIRRWNLG

SEQ ID NO: 260 - protein, 37 aa, Medicago truncatula
NFECKSTNDCPKSVLRVWRCINNYCRPVRMKKIHNTM

SEQ ID NO: 261 - protein, 35 aa, Medicago truncatula
NRVVVTIGGNCIRDKDCLKFYGANIRCRKGKCVAI

SEQ ID NO: 262 - protein, 43 aa, Medicago truncatula
AFVPNSGPCTTDKDCKQVKGYIARCRKGYCMQSVKRTWSSYSR

SEQ ID NO: 263 - protein, 38 aa, Medicago truncatula
IYFRKPPPCITDKDCPQMKINNVRCRKGFCIQIHKFTP

SEQ ID NO: 264 - protein, 39 aa, Medicago truncatula
CDSIYYPISRPCKTDKDCPNRKNYKGKCRKGFCMSSRLR

SEQ ID NO: 265 - protein, 33 aa, Medicago truncatula
MKYGCKHTGHCPRKMCGAKTTKCRNNKCQCVQL

SEQ ID NO: 266 - protein, 36 aa, Medicago truncatula
AYLPFSRSCISDKDCPQVKNFSARCRKGYCQFRVVR

SEQ ID NO: 267 - protein, 40 aa, Medicago truncatula
SSPIRVTDVSRPCTTDKDCSKVEYGYKLRCRKGRCVHIPR

SEQ ID NO: 268 - protein, 35 aa, Medicago truncatula
FPWKLYPCVTDKDCPRKNRHVVKCRKGYCVGVQII

SEQ ID NO: 269 - protein, 36 aa, Medicago truncatula
APLMLKPCITDKDCPKHRGVNGKCRKGYCVGVGRNY

SEQ ID NO: 270 - protein, 90 aa, Medicago truncatula
YVIIIFLSLFLVATNIEGKLFYRFQISFFYFGHNILLYFCYLILFSFFITTAFFRCKNDFDCVHKRCRGPMRAKCISKAICKCRLAFTLK

SEQ ID NO: 271 - protein, 36 aa, Medicago truncatula
YKTKTPCKSLNDCPKAIKPIFVKCLGNICQYSIGRI

SEQ ID NO: 272 - protein, 77 aa, Medicago truncatula
ARRKNFFFIILKFSSLLTQYFMALNKMALFLFYYIGIRRLNCNSDDGCPNNYCTPPHFGKCVSKQCVCMKLAWYTKP

SEQ ID NO: 273 - protein, 52 aa, Medicago truncatula
KKVYVTIFCYLFFVTAYHLCKTRFDCPRTYLLFFPRMWKCINRRCRYVYFFE

SEQ ID NO: 274 - protein, 37 aa, Medicago truncatula
KPGSPNLRNHSCLRDKDCPQLKFHNIRCRNGFCVDIR

SEQ ID NO: 275 - protein, 36 aa, Medicago truncatula
TYLPTTRICITDKDCPSVKNYIGRCRKGYCQASKLR

SEQ ID NO: 276 - protein, 50 aa, Medicago truncatula
KNIDGRVSYNSFIALPVCQTAADCPEGTRGRTYKCINNKCRYPKLLKPIQ

SEQ ID NO: 277 - protein, 30 aa, Medicago truncatula
QVKCKTVKDCPIRRNRKYYCLFGICKYDVM

SEQ ID NO: 278 - protein, 31 aa, Medicago truncatula
QIMFSDCKTDKDCPQFRRANIRCRKGQCVKL

SEQ ID NO: 279 - protein, 34 aa, Medicago truncatula
SFSQIFNSACKTDKDCPKFGRVNVRCRKGNCVPI

SEQ ID NO: 280 - protein, 39 aa, Medicago truncatula
GGKKIYCENAASCPRLMYPLVYKCLDNKCVKFMMKSRFV

SEQ ID NO: 281 - protein, 39 aa, Medicago truncatula
LKKCITFEDCPISKTRVYKCLHGECRYTIPYIPKVPKVK

SEQ ID NO: 282 - protein, 33 aa, Medicago truncatula
FSKTFDRACKTDKDCPKLRGVNVRCRKDQCVTV

SEQ ID NO: 283 - protein, 34 aa, Medicago truncatula
SPGRLSFGDCKIDKDCPKLRGGSFRCRKGKCVLE

SEQ ID NO: 284 - protein, 36 aa, Medicago truncatula
SFLPNSRTCITDKDCPNGRNYIGRCRKGQCQQSPLR

SEQ ID NO: 285 - protein, 36 aa, Medicago truncatula
SFFPSSPVCKTDKDCPQLRGYTARCRKTQCLLIPRG

SEQ ID NO: 286 - protein, 26 aa, Medicago truncatula
VSKIICVLSQKPLCRNHICVCYKLNR

SEQ ID NO: 287 - protein, 34 aa, Medicago truncatula
TTPTPCRTDQDCPRKKKFSVTCRKGFCAEIRHVY

SEQ ID NO: 288 - protein, 39 aa, Medicago truncatula
SPLQRPCVTVADCPPVKKPLKMWCIRQTCFYGFGKRPDL

SEQ ID NO: 289 - protein, 34 aa, Medicago truncatula
FLGTFISSCKRDKDCPKLYGANFRCRKGTCVPPI

SEQ ID NO: 290 - protein, 31 aa, Medicago truncatula
QIIFSECKTDKDCPKYQRANIRCRKGQCVRI

SEQ ID NO: 291 - protein, 36 aa, Medicago truncatula
ITYFDVRCEKDKDCPKPPRINIRINIRCRKGFCVQI

SEQ ID NO: 292 - protein, 47 aa, Medicago truncatula
LNGHGKDRCFKDSDCPKYMCPSSLVAKCIKKLCSCRKPGLQIQLNPK

SEQ ID NO: 293 - protein, 32 aa, Medicago truncatula
FPLMPIPCRTDKDCPKKMGTVGKCRKGYCAQM

SEQ ID NO: 294 - protein, 36 aa, Medicago truncatula
NSAFSHFIPGCKTDKDCPKFYGSNVRCRKGKCVQLG

SEQ ID NO: 295 - protein, 35 aa, Medicago truncatula
YIPGIVNKPCKTDKDCPKKPPHNIRCRKGQCVEIL

SEQ ID NO: 296 - protein, 47 aa, Medicago truncatula
SNTLLAFRECVYDKDCPVMPRCNMRCRKGVCIPVRRKEYFKMNVSLL

SEQ ID NO: 297 - protein, 59 aa, Medicago truncatula
AYFECHSDSACETTVKCVLPRIPRCIKYKCLCGNGVGKRWSTTPKRIEKGSTVRNGFLH

SEQ ID NO: 298 - protein, 44 aa, Medicago truncatula
SFTRCFRDSNCPKSLCHPPTKPKCMYKSICKCIREFSKRDYVHT

SEQ ID NO: 299 - protein, 38 aa, Medicago truncatula
TPQKKVTDVSHPCTTDKDCPKVQHGYKLRCRKGQCVHI

SEQ ID NO: 300 - protein, 36 aa, Medicago truncatula
FYSHVPNPCITDKDCIRRAGMNIRCRKGYCVNLILR

SEQ ID NO: 301 - protein, 36 aa, Medicago truncatula
SFIFMPCITDKDCPALKKNKGKGRCRKGYCVDGLIG

SEQ ID NO: 302 - protein, 91 aa, Medicago truncatula
VRLLQIVKSISIKCRSNQCLFLRYGFRLASIKYIFITNSEIWLFQTTEYSHICRLMYFTILGYEHECYEFVRKFIIFVFSEFVLYRRTLTI

SEQ ID NO: 303 - protein, 36 aa, Medicago truncatula
TYIPITRTCITDKDCPSWKNYTGRCRKGFCILNRDR

SEQ ID NO: 304 - protein, 69 aa, Medicago truncatula
GIKFNGECRNINDCYRKYTLVPYGYMRCIRGYCKLQRDVKVVFSVKEWKEIPYMKTLFHVFYIWGCGHL

SEQ ID NO: 305 - protein, 36 aa, Medicago truncatula
SYIPISHPCTTVKDCPEVKNYKSRCLKGLCISGRLR

SEQ ID NO: 306 - protein, 38 aa, Medicago truncatula
DSFNPLIRQYCVTDKDCPKFKKYNIRCRKGFCVQVNGG

SEQ ID NO: 307 - protein, 39 aa, Medicago truncatula
DGVKNINRECTQTSDCYKKYPFIPWGKVRCVKGRCRLDM

SEQ ID NO: 308 - protein, 45 aa, Medicago truncatula
KFHRCFKDSECLNLLYCRTPLKPKCMYRTFCKCKVVFTSNVYVLT

SEQ ID NO: 309 - protein, 31 aa, Medicago truncatula
RVCKSDKDCKDIIIYRYILKCRNGECVKIKI

SEQ ID NO: 310 - protein, 32 aa, Medicago truncatula
YVVCITNNDCPPRTRLILYKCRNRKCVSYSII

SEQ ID NO: 311 - protein, 41 aa, Medicago truncatula
YPPCETVADCPESYFRIYRCENNFCRYREAVRRLRPPLRKK

SEQ ID NO: 312 - protein, 60 aa, Medicago truncatula
MVETLKLVYVLNLFYSIFLGIIVCNTSLIKYFDVPWEKDKDCPKPLRFNIRCRKGFCVRI

SEQ ID NO: 313 - protein, 44 aa, Medicago truncatula
YYYKCFKDSDCVKLLCRIPLRPKCMYRHICKCKVVLTQNNYVLT

SEQ ID NO: 314 - protein, 35 aa, Medicago truncatula
AYLPLSRSCITDKDCSRVKNYNARCRKGYCQYLQY

SEQ ID NO: 315 - protein, 51 aa, Medicago truncatula
GCPSLLRVGRKSMLARITICTGERDCRSYHCIPHEFPRCIRGMCRCKYSGN

SEQ ID NO: 316 - protein, 36 aa, Medicago truncatula
HKRCRVDFDCRMRMCVYPTVSVCIDRLCRCRRPPNM

SEQ ID NO: 317 - protein, 41 aa, Medicago truncatula
INKCSQDSHCPKDMCKKPSKPRCVVSPKLPLSSKSGVCTCV

SEQ ID NO: 318 - protein, 49 aa, Medicago truncatula
QNIDSGGNRRCFRDSDCPKNMCPSYLVVKCLRSNCKCVRPGLQVRLNPN

SEQ ID NO: 319 - protein, 44 aa, Medicago truncatula
QKRRRSTECRNDSDCEKMVKCVLPRIARCIKYRCQCRNFLESFE

SEQ ID NO: 320 - protein, 34 aa, Medicago truncatula
AVRKPECRQNSDCPPYFCIKPTVPKCIKFKCLCK

SEQ ID NO: 321 - protein, 59 aa, Medicago truncatula
YDDCYNHTECTNKIKCVPPRIAQCFRFKCDCIRLNNGPKTPWSATPKRVHISPTRKNDF

SEQ ID NO: 322 - protein, 61 aa, Medicago truncatula
AHVECHNDSACEKTVKCMLPRIPRCIKYQCLCGYSDDPGNRWSTRPKRIQKGSTERKGFLY

SEQ ID NO: 323 - protein, 45 aa, Medicago truncatula
ERIYRCLDHSHCPTFMCSPGLKPKCMNPKVCKCVPVQSRKYYALT

SEQ ID NO: 324 - protein, 74 aa, Medicago truncatula
NQFLIFLTFLSYSVHNIIYYFNNIFLFSFFITAINKCSQDSHCPKDMCKKPSKPRCVVSPKLPLSSKSGVCTCV

SEQ ID NO: 325 - protein, 59 aa, Medicago truncatula
AYYECSNDSACQATTKCVLPRVPRCIKYKCLCGNSNGSGNRWSTRPNRIQKGSTESNYF

SEQ ID NO: 326 - protein, 37 aa, Medicago truncatula
SFSQMINFRGCKRDKDCPQFRGVNIRCRSGFCTPIDS

SEQ ID NO: 327 - protein, 36 aa, Medicago truncatula
NSSFSHFFEGACKSDKDCPKLHRSNVRCRKGQCVQI

SEQ ID NO: 328 - protein, 38 aa, Medicago truncatula
NQNNNPKNNNPWACISDKDCQKVHGVNIRCRKGFCTVI

SEQ ID NO: 329 - protein, 38 aa, Medicago truncatula
VSNSSFSEIIDSACKTDKDCPKLHKVNVRCRKGKCVAI

SEQ ID NO: 330 - protein, 36 aa, Medicago truncatula
AFVANTETCITDKDCPNGRNYIGRCRKGHCQQRLVR

SEQ ID NO: 331 - protein, 34 aa, Medicago truncatula
NNSPPCVTDKDCPRPQFRKSNVRCRNGYCVNLGN

SEQ ID NO: 332 - protein, 40 aa, Medicago truncatula
KRNLPECSTDDHCKQVSSKFYRHICHNNRCKIVMFMPPNV

SEQ ID NO: 333 - protein, 33 aa, Medicago truncatula
NPYIINILCKTDKDCPKVQGANIRCRSGKCVQV

SEQ ID NO: 334 - protein, 23 aa, Medicago truncatula
KVLKCTPPEVVKCTCLCGKRTLY

SEQ ID NO: 335 - protein, 64 aa, Medicago truncatula
MQRAKKMVETLKFIYVMILFVSIFLVMIVCDFSNLHIHVPCLTDKDCPWAKNYVLKCRKGYCVF

SEQ ID NO: 336 - protein, 37 aa, Medicago truncatula
SNYSPTPFPCLTDKDCTRRKGFSVTCRKGFCVEFKHF

SEQ ID NO: 337 - protein, 33 aa, Medicago truncatula
LNLADYSCIKDKDCPKVKSFNIRCRKGFCVNIL

SEQ ID NO: 338 - protein, 35 aa, Medicago truncatula
FIFLPCITDKDCPSLEKNKGKGRCRKGYCVHGLIG

SEQ ID NO: 339 - protein, 31 aa, Medicago truncatula
APKPCVTVKDCPKTVFPIGYKCIKNLCILVI

SEQ ID NO: 340 - protein, 36 aa, Medicago truncatula
TYLPSTRSCITDKDCPNVQNYIGRCRKGFCISSWVR

SEQ ID NO: 341 - protein, 60 aa, Medicago truncatula
NPQGTCYTFVHCYIKNGWPFKGVWRCNKGLLLVQLIMTMCVLLIHIRIVLEKMLLLDVTP

SEQ ID NO: 342 - protein, 49 aa, Medicago truncatula
LILFISTFLVIIVYDSKTFYFSLPCKIDKDCPRNPPLNIRCRKSFCVQI

SEQ ID NO: 343 - protein, 36 aa, Medicago truncatula
YFNDPRPCVSDKDCPRPKFQKSNVRCRKGYCVNLDG

SEQ ID NO: 344 - protein, 47 aa, Medicago truncatula
IRTYRECENASDCYSIYWRAPYGTMRCVKGHCKQIKDVKVMKFLYCV

SEQ ID NO: 345 - protein, 32 aa, Medicago truncatula
FPFMPIPCLTDKDCPKKNGYVGKCRKGYCVGI

SEQ ID NO: 346 - protein, 43 aa, Medicago truncatula
VYDSKYFNIAPPCVIDKDCPQFKNNNVRCRKGFCVNLSVNWIK

SEQ ID NO: 347 - protein, 46 aa, Medicago truncatula
FIDCETVADCPTHWAYIYVCEKNKCRYHFKSGRVRPDHQKNRHNRV

SEQ ID NO: 348 - protein, 49 aa, Medicago truncatula
VSITGDISGNLARASRKKPVDVIPCIYDHDCPRKLYFLERCVGRVCKYL

SEQ ID NO: 349 - protein, 40 aa, Medicago truncatula
SVYDSIPYVNSGPCVTDKDCPKVSQYNIRCRKGQCARIRV

SEQ ID NO: 350 - protein, 36 aa, Medicago truncatula
IHFHVSRPCMTDNDCAPEKYYNIRCRKGFCVQIRKY

SEQ ID NO: 351 - protein, 40 aa, Medicago truncatula
WSWGLTTECVTELDCYKKYRLPAEKKMKCIRGSCYRVAHF

SEQ ID NO: 352 - protein, 57 aa, Medicago truncatula
ISIYVRCASTNECYTTFKFAPLGSMRCVEGYCKHLKDFKVKTPLQIKEITPLLLHFP

SEQ ID NO: 353 - protein, 54 aa, Medicago truncatula
DKPRYTPRNAVKIAECVSYTDCQGGCPACYMRLTWFFKYILFFLSVHGLPSRNS

SEQ ID NO: 354 - protein, 39 aa, Medicago truncatula
IRIHCKDDFDCIENRLQVGCRLQREKPRCVNLVCRCLRR

SEQ ID NO: 355 - protein, 43 aa, Medicago truncatula
GIRCVFPSDCPRTMRCLSEFHLTCKKKQCKCVKMFDPINFVTA

SEQ ID NO: 356 - protein, 46 aa, Medicago truncatula
LNGGGKDKCFRDSDCPKHMCPSSLVAKCINRLCRCRRPELQVQLNP

SEQ ID NO: 357 - protein, 40 aa, Medicago truncatula
LPISCKDHFECRRKINILRCIYRQEKPMCINSICTCVKLL

SEQ ID NO: 358 - protein, 56 aa, Medicago truncatula
LLEKGRRRNMVEIVKCNVDSDCPKNIWYCETGRINRCIRHQCRCVKLISPVDFVST

SEQ ID NO: 359 - protein, 38 aa, Medicago truncatula
QIFPKWCLYDKDCPQNMCRPGRIPKCIFGHCNCVKQRS

SEQ ID NO: 360 - protein, 33 aa, Medicago truncatula
SATTRCVRNSDCRHHICMYPLVPRCKYPLCRCV

SEQ ID NO: 361 - protein, 42 aa, Medicago truncatula
IIRCFHDADCVHKICHPPQIRKCVSKICKCRLMITQKDYVLT

SEQ ID NO: 362 - protein, 39 aa, Medicago truncatula; identical to SEQ ID NO: 229

SEQ ID NO: 363 - protein, 31 aa, Medicago truncatula
TTKCKNNFECRFLICMNPLVPKCKVPYCRCM

SEQ ID NO: 364 - protein, 37 aa, Medicago truncatula
YFPLTVHILCKHDRNCPKNMCLLTQIPKCFKNVCKCV

SEQ ID NO: 365 - protein, 37 aa, Medicago truncatula
GNYKCQTNYDCLRMWCPIGISPRCIKRRCKCIETLVQ

SEQ ID NO: 366 - protein, 42 aa, Medicago truncatula
GIRCRNVYDCPKATYCRAGSHRVQCIKHQCKCVRIFESIDPA

SEQ ID NO: 367 - protein, 51 aa, Medicago truncatula
VYDSKYFQIASPCVNDKDCPQFKNNNVRCRRGFCVNSGGATQKCLGCPSLK

SEQ ID NO: 368 - protein, 40 aa, Medicago truncatula
TAIHECRAHSHCVARINCVLPRKPQCRNYACGCYDSNKYR

SEQ ID NO: 369 - protein, 41 aa, Medicago truncatula
YKNRCFRDSDCPKEMCNHPKIPKCVNNAYCKCVVAMYFPPK

SEQ ID NO: 370 - protein, 39 aa, Medicago truncatula
ARYECREDSHCVTKVKCGLPRTPKCRNYICFCHNPNKYI

SEQ ID NO: 371 - protein, 68 aa, Medicago truncatula
RVKYKEDYGGQNKWNEFPLKPHIRTYRIKCKDDSGCEGNNLCEGRWIPKCLKPYFLFLTTKEGFCACV

(19) United States
(12) Patent Application Publication
(10) Pub. No.: US 2026/0297146 A1
(43) Pub. Date: Oct. 1, 2026

(54) ANTIMICROBIAL NODULE-SPECIFIC CYSTEINE-RICH PEPTIDE FOLDING VARIANTS

(71) Applicant: Donald Danforth Plant Science Center, St. Louis, MO (US)

(72) Inventors: Dilip M. Shah, Chesterfield, MO (US); Meenakshi Tetorya, St. Louis, MO (US)

(21) Appl. No.: 19/477,288

(22) PCT Filed: Apr. 23, 2024
(86) PCT No.: PCT/US2024/025777

§ 371 Date: Oct. 21, 2025

Related U.S. Application Data
(60) Provisional application No. 63/498,059, filed on Apr. 25, 2023.

(51) Int. Cl. C07K 14/47; A01N 37/46; A01P 1/00; A61K 38/00; A61L 27/22; C12N 9/76; C12P 21/02

(52) U.S. Cl. CPC C07K 14/4723; A01N 37/46; A01P 1/00; A61L 27/227; C12N 9/6427; C12P 21/02; A61K 38/00

(57) ABSTRACT

Provided are compositions and methods employing antimicrobial nodule-specific cysteine-rich (NCR) peptides and variants thereof, including NCR peptide folding variants (NCR_PFV) including folding variants of antimicrobial NCR peptides and proteins (NCR_PFV) that exhibit favorable antifungal properties and a desirable resistance or susceptibility to protease cleavage, which antimicrobial NCR_PFV can be applied ex vivo directly to a plant, human, or animal or can be administered in vivo to a plant, human, or animal.

### CROSS-REFERENCE TO RELATED APPLICATIONS

This application claims priority under 35 U.S.C. § 119 to provisional patent application U.S. Ser. No. 63/498,059, filed Apr. 25, 2023. The provisional patent application is herein incorporated by reference in its entirety, including without limitation, the specification, claims, and abstract, as well as any figures, tables, appendices, or drawings thereof.

### SEQUENCE LISTING

The instant application contains a Sequence Listing which has been submitted electronically in XML format and is herein incorporated by reference in its entirety. Said XML copy, created on Apr. 19, 2024 is named “P13860WO01_SequenceListing.xml” and is 333,911 bytes in size.

### TECHNICAL FIELD

The present disclosure relates, generally, to antimicrobial peptides and proteins for use in controlling pathogenic microbes. Provided herein are compositions and methods employing antimicrobial nodule specific cysteine rich (NCR) peptides and proteins, including folding variants of antimicrobial NCR peptides and proteins (NCR_PFV) that exhibit favorable antifungal properties and/or a desirable resistance or susceptibility to protease cleavage, which antimicrobial NCR_PFV can be applied ex vivo directly to a plant, human, or animal or can be administered in vivo to a plant, human, or animal.

### BACKGROUND OF THE DISCLOSURE

Animals and plants are both susceptible to microbial infections that, if left untreated, can result in substantial morbidity and mortality in human and veterinary patients and loss of food crops, which are particularly susceptible to contamination and spoilage during storage. Protection of agriculturally important crops from pathogenic microbes is crucial in improving crop yields. Fungal infections are a particular problem in damp climates and can become a major concern during crop storage, where such infections can result in spoilage and contamination of food or feed products with fungal toxins. Unfortunately, modern growing methods, harvesting and storage systems can promote plant pathogen infections.

The control of pathogens in humans, veterinary animals, and plants is complicated by the need for simultaneous control of multiple pathogenic microbes of distinct genera, including fungi, dimorphic fungi, yeast, molds, and oomycetes. For example, plants are susceptible to infection by a wide range of microbes including Alternaria, Ascochyta, Botrytis, Cercospora, Colletotrichum, Diplodia, Erysiphe, Fusarium, Gaeumanomyces, Helminthosporium, Macrophomina, Magnaporthe, Nectria, Peronospora, Phoma, Phakopsora, Phymatotrichum, Phytophthora, Plasmopara, Podosphaera, Puccinia, Pythium, Pyrenophora, Pyricularia, Rhizoctonia, Sclerotium, Sclerotinia, Septoria, Thielaviopsis, Uncinula, Venturia, and Verticillium species are all recognized plant pathogens.

Consequently, antimicrobial agents that control a limited subset of microbial pathogens provide inadequate or ineffective protection under conditions where multiple pathogens are present. Moreover, in many cases, plant pathogenic microbes have become resistant to existing antimicrobial agents.

Antimicrobial peptides are reviewed in Hancock, AAC 43(6):1317 (1999) and Yount, PNAS 101(19):7363 (2004). Antimicrobial peptides include nodule-specific cysteine-rich (NCR) peptides and Defensin peptides.

Defensins are small cysteine-rich peptides of about 45-54 amino acids that constitute an important component of the innate immunity of plants (Sathoff, Phytopathology 109:402 (2019)). PCT Patent Publication No. WO 2010/146,067 describes antimicrobial nodule specific cysteine rich (NCR) peptides from Medicago truncatula (Barrel Medic). Montiel et al., Molec. Plant Microb. Inter. 29:210-219 (2016) describes NCR peptides from Cicer arietinum (Chickpea) and implicates those NCR peptides in the terminal differentiation of endosymbiotic bacteria. Anti-microbial and anti-oomycete activity of certain NCR peptides has been disclosed in WO2020/146360. Certain NCR peptides exhibit antimicrobial properties when applied to free-living bacteria and can mediate bacterial cell death and early nodule senescence. Yang, PNAS 114:6848-6853 (2017) and Wang, PNAS 114:6854-6859 (2017). Antimicrobial NCRs (AMPs) are cationic and have conserved cysteine residues that form intramolecular disulfide bonds. Cysteine substitutions or disulfide bond modifications can influence the antimicrobial activity of certain NCR peptides having just 4 conserved cysteine residues. Haag, J. Biol. Chem. 287(14):10791-8 (2012) and Isozumi, Nature Sci. Rep. 11: 9923 (2021).

Nodule-specific cysteine-rich (NCR) peptides are normally expressed in the root nodules of leguminous plants, including Cicer arietinum, Medicago truncatula, Galega orientalis, Medicago sativa, Astragalus canadensis, Pisum sativum, Ononis spinosa, Onobrychis viciifolia, and Oxytropis lambertii where they mediate the differentiation of root nodule bacteria into nitrogen-fixing bacteroids thereby maintaining bacterial survival. Wang, Mol. Plant-Microbe Int. 31(2):240-8 (2018), Van de Velde, Science 327:1122-1126 (2010), Kim, PNAS 112:15238-15243 (2015), and Horvath, PNAS 112:15232-15237 (2015).

Despite recent advancements in the control and treatment of microbial infections and the recognition that certain antimicrobial peptides exhibit substantial antimicrobial properties and activities, there remains a substantial unmet need for therapeutically effective modalities for the prevention, treatment, and control of pathogenic microorganisms.

### SUMMARY OF THE DISCLOSURE

The present disclosure is based upon the discovery that certain cationic antimicrobial nodule-specific cysteine-rich (NCR) peptides and proteins can form alternative disulfide bonding pairs and, consequently, can fold into multiple distinct conformations each having a unique secondary (e.g., alpha helices and beta sheets) and tertiary structure, and which NCR peptide and protein structural variants possess distinct antimicrobial activities and resistance to proteolytic cleavage. As disclosed herein, an NCR peptide folding variant that exhibit a desired level of antimicrobial activity and protease resistance can be purified from a heterogeneous mixture containing multiple (i.e. two or more) NCR peptide folding variants and the purified NCR peptide folding variant can be used advantageously in the compositions and methods disclosed herein. Thus, within various aspects, the present disclosure provides nodule-specific cysteine rich NCR peptide folding variants 1 (NCR_PFV1), compositions comprising antimicrobial NCR_PFV1, methods for making antimicrobial NCR_PFV1 compositions, methods for using such antimicrobial NCR_PFV1 compositions, and devices, including medical devices, which comprise an antimicrobial NCR_PFV1.

In certain embodiments, provided herein are compositions comprising cationic 6-Cys nodule-specific cysteine-rich (NCR) peptide folding variants 1 (NCR_PFV1) wherein the NCR peptide comprises in contiguous amino acid sequence from its N-terminal end (1) a first cysteine pair motif including C₁ and C₂, (2) a first intervening sequence (IS1), (3) a second cysteine pair motif including C₃ and C₄, (4) a second intervening sequence (IS2), and (5) a third cysteine pair motif including C₅ and C₆, wherein the NCR peptide has less than 60%, 70%, 80%, 85%, 90%, 92%, 95%, 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4), wherein the cysteine residues C₁, C₂, C₃, C₄, C₅, and C₆ of NCR_PFV1 form a first set of disulfide bonds and NCR_PFV1 thereby adopts a first conformation that exhibits greater antimicrobial activity as compared to a nodule-specific cysteine rich NCR peptide folding variant 2 (NCR_PFV2) that has the same amino acid sequence as NCR_PFV1 but forms a distinct second set of disulfide bonds between cysteine residues C₁, C₂, C₃, C₄, C₅, and C₆ and adopts a distinct second conformation, and wherein the composition is substantially free of NCR_PFV2, optionally wherein at least about 80%, 90%, 95%, 98%, 99%, or 99.5% of the total NCR peptide in the composition by weight is NCR_PFV1.

In related embodiments, provided herein are compositions comprising cationic 4-Cys nodule-specific cysteine-rich (NCR) peptide folding variants 1 (NCR_PFV1) wherein the NCR peptide comprises in contiguous amino acid sequence from its N-terminal end (1) a first cysteine pair motif including C₁ and C₂, (2) a first intervening sequence (IS1), and (3) a second cysteine pair motif including C₃ and C₄, wherein the NCR peptide has less than 60%, 70%, 80%, 85%, 90%, 92%, 95%, 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4), wherein the NCR_PFV1 cysteine residues C₁, C₂, C₃, and C₄ form a first set of disulfide bonds and NCR_PFV1 thereby adopts a first conformation that exhibits greater antimicrobial activity as compared to a nodule-specific cysteine rich NCR peptide folding variant 2 (NCR_PFV2) that has the same amino acid sequence as NCR_PFV1 but forms a distinct second set of disulfide bonds between cysteine residues C₁, C₂, C₃, and C₄ and adopts a distinct second conformation, and wherein the composition is substantially free of NCR_PFV2, optionally wherein at least about 80%, 90%, 95%, 98%, 99%, or 99.5% of the total NCR peptide in the composition by weight is NCR_PFV1.

In other embodiments, provided herein are methods for making compositions comprising cationic antimicrobial 6-Cys or 4-Cys nodule-specific cysteine-rich (NCR) peptide folding variants 1 (NCR_PFV1), which methods comprise: (a) separating a fraction comprising NCR peptide folding variant 1 (NCR_PFV1) from one or more fractions comprising NCR peptide folding variant 2 (NCR_PFV2), or NCR_PFV2 peptide fragments thereof, from a mixture comprising NCR_PFV1 and NCR_PFV2, or NCR_PFV2 peptide fragments thereof; and (b) combining the fraction comprising NCR_PFV1, or a preparation further purified from the NCR_PFV1 fraction, with an agriculturally, pharmaceutically, or veterinary practicable carrier, diluent, or excipient, wherein the NCR peptide has less than 60%, 70%, 80%, 85%, 90%, 92%, 95%, 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4), thereby making the composition.

In further embodiments, provided herein are methods for preventing or reducing crop damage or post-harvest loss by a plant pathogenic microbe, the method comprising contacting a plant, a plant seed, pre- or post-harvest grain, pre- or post-harvest fruit, or pre- or post-harvest vegetable with an effective amount of a composition comprising a cationic antimicrobial 6-Cys or 4-Cys nodule-specific cysteine-rich (NCR) peptide folding variant 1 (NCR_PFV1) under conditions that are suitable for preventing or reducing crop damage or post-harvest loss, wherein the NCR peptide has less than 60%, 70%, 80%, 85%, 90%, 92%, 95%. 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4).

In yet other embodiments, provided herein are medical devices in operable combination with a composition comprising a cationic antimicrobial 6-Cys or 4-Cys nodule-specific cysteine-rich (NCR) peptide folding variant 1 (NCR_PFV1), wherein the medical device comprises at least one surface that is topically coated or impregnated with the composition and wherein the NCR peptide has less than 60%, 70%, 80%, 85%, 90%, 92%, 95%, 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4).

In still further embodiments, provided herein are methods for treating, preventing, or inhibiting a microbial infection in a subject (including a plant, human, or non-human animal) in need thereof, which methods comprise administering (ex vivo or in vivo) to the subject a therapeutically effective amount of a composition comprising a cationic antimicrobial 6-Cys or 4-Cys nodule-specific cysteine-rich (NCR) peptide folding variant 1 (NCR_PFV1) under conditions and for a duration suitable for treating, preventing, or inhibiting the microbial infection, wherein the NCR peptide has less than 60%, 70%, 80%, 85%, 90%, 92%, 95%, 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4).

In related embodiments, provided herein are compositions comprising a cationic antimicrobial 6-Cys or 4-Cys nodule-specific cysteine-rich (NCR) peptide folding variant 1 (NCR_PFV1) for use in a method for treating, preventing, or inhibiting microbial infection in a subject in need thereof, wherein the NCR peptide has less than 60%, 70%, 80%, 85%, 90%, 92%, 95%, 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4).

In other related embodiments, provided herein are plant parts that are at least partly coated with a composition comprising a cationic antimicrobial 6-Cys or 4-Cys nodule-specific cysteine-rich (NCR) peptide folding variant 1 (NCR_PFV1), wherein the NCR peptide has less than 60%, 70%, 80%, 85%, 90%, 92%, 95%, 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4).

These and other aspects of the present disclosure will be better understood in light of the following drawings and detailed description, which exemplify certain aspects of the various embodiments.

### DETAILED DESCRIPTION

In certain embodiments, the present disclosure provides compositions enriched for cationic and antimicrobial 6-Cys and 4-Cys nodule-specific cysteine rich (NCR) peptide folding variants (NCR_PFV) having a defined set of disulfide bonding pairs, adopting a distinct conformation having unique tertiary structures, possessing improved antimicrobial activities, and, optionally, exhibiting a desired resistance or sensitivity to proteolytic cleavage in comparison to other NCR_PFV with different disulfide bonding pairs. In certain embodiments, the present disclosure provides cationic and antimicrobial 6-Cys and 4-Cys nodule-specific cysteine rich (NCR) peptide folding variants (NCR_PFV) having a defined set of disulfide bonding pairs, exhibiting a desired resistance or sensitivity to proteolytic cleavage in comparison to other NCR_PFV with different disulfide bonding pairs. As disclosed herein, cationic and antimicrobial 6-Cys and 4-Cys NCR_PFV can be purified from heterogeneous mixtures containing multiple (i.e. two or more) NCR folding variants and the desired purified NCR_PFV can be used advantageously in the compositions, methods, and devices as disclosed herein. The aforementioned and after mentioned antimicrobial 6-Cys and 4-Cys nodule-specific cysteine rich (NCR) peptides which are the object of this disclosure are peptides which have less than less than 60%, 70%, 80%, 85%, 90%, 92%, 95%, 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4).

These and other aspects of the present disclosure can be better understood by reference to the following non-limiting definitions.

### Definitions

As used herein, the terms “microbe,” “microbes,” and “microbial” are used to refer to bacteria, fungi (including yeast), and oomycetes.

As used herein, the phrases “susceptible microbe (or microbes),” “susceptible microbial infection,” and the like refer to microbes that infect plants, humans, and non-human animals, or microbial infections thereof, that are subject to inhibition of microbial growth by antimicrobial peptides, including nodule-specific cysteine-rich (NCR) and Defensin peptides, variants, and multimers thereof.

As used herein, the phrases “combating microbial damage,” “combating or controlling microbial damage,” or “controlling microbial damage” refer to reduction in damage to a plant, such as an ornamental plant or a crop plant, or to an ornamental plant or crop plant product due to infection by a microbial pathogen. More generally, these phrases refer to reduction in the adverse effects caused by the presence of a pathogenic microbe in the crop plant. Adverse effects of microbial growth are understood to include any type of plant tissue damage or necrosis, any type of plant yield reduction, any reduction in the value of the crop plant product, and/or production of undesirable microbial metabolites or microbial growth by-products including to mycotoxins.

As used herein, the phrases “inhibiting growth of a plant pathogenic microbe,” “inhibit microbial growth,” and the like refer to methods that result in any measurable decrease in microbial growth, where microbial growth includes but is not limited to any measurable decrease in the numbers and/or extent of microbial cells, spores, conidia, or mycelia. As used herein, “inhibiting growth of a plant pathogenic microbe” is also understood to include any measurable decrease in the adverse effects cause by microbial growth in a plant. Adverse effects of microbial growth in a plant include any type of plant tissue damage or necrosis, any type of plant yield reduction, any reduction in the value of the crop plant product, and/or production of undesirable microbial metabolites or microbial growth by-products including but not limited to mycotoxins. As used herein, the phrase “inhibition of microbial growth” and the like, unless otherwise specified, can include inhibition in a plant, human or animal.

As used herein, the term “antimicrobial peptide” as used herein refer to peptides, in particular nodule-specific cysteine-rich (NCR) peptides, which exhibit any one or more of the following characteristics of inhibiting the growth of microbial cells, killing microbial cells, disrupting or retarding stages of the microbial life cycle such as spore germination, sporulation, or mating, and/or disrupting microbial cell infection, penetration or spread within a plant or other susceptible subject, including a human, livestock, poultry, fish, or a companion animal (e.g., dog or cat).

As used herein, the phrase “cation-tolerant” refers to an NCR peptide or a variant thereof that exhibits equivalent in vitro antifungal or antimicrobial activity or no more than about a 1.5-, 2-, 3-, or 4-fold decrease in in vitro antifungal or antimicrobial activity in the presence of 100 mM KCl or 100 mM NaCl as compared to the antifungal activity of the NCR peptide or the variant thereof in the absence of KCl or NaCl.

As used herein, the phrase “consensus sequence” refers to an amino acid sequence created by aligning two or more homologous sequences and deriving a new sequence having the conserved set of alternative amino acid residues of the homologous sequences at each position in the peptide sequence.

As used herein, the term “peptide variant” refers to any peptide with antimicrobial activity comprising one or more non-conservative amino acid substitutions in a nodule-specific cysteine-rich (NCR) peptide. An NCR peptide variant can, in addition to such non-conservative substitutions, further comprise a peptide having conservative amino acid substitutions, deletions of one to five amino acids from the N-terminus, and internal deletions of one or more amino acid residues in an NCR peptide variant provided herein.

The phrases “percent identity” or “sequence identity” as used herein refer to the number of elements (i.e., amino acids or nucleotides) in a sequence that are identical within a defined length of two protein segments in an alignment resulting in the maximal number of identical elements, and is calculated by dividing the number of identical elements by the total number of elements in the defined length of the aligned segments and multiplying by 100.

As used herein, the term “endoproteinase” refers to a peptidase that cleaves a peptide bond between two internal amino acid residues in a peptide sequence. Endoproteinases can also be referred to as “endoproteases” or “endopeptidases.” The proteolytic activity of an endoproteinase, endoprotease, or endopeptidase is thus different that the proteolytic activity of an “exopeptidase,” which cleaves peptide bonds of terminal amino acid residues in a peptide.

As used herein, the term “heterologous peptide” or “heterologous protein” refers to peptide, such a nodule-specific cysteine-rich (NCR) peptide that is not found in a cell or organism as the cell or organism occurs in nature. As such, heterologous peptides include peptides that are localized in a subcellular location, extracellular location, or expressed in a tissue that is distinct from the subcellular location, extracellular location, or tissue where the peptide or protein is found in a cell or organism as it occurs in nature.

As used herein, the term “amino acid” refers to an organic compound that contains amino (—NH₃) and carboxylate (—CO₂) functional groups, along with a side chain (R group) specific to each amino acid. In polypeptides are in certain instance referred to herein by one letter amino acid codes as follows: G—Glycine (Gly); P—Proline (Pro); A—Alanine (Ala); V—Valine (Val); L—Leucine (Leu); I—Isoleucine (Ile); M—Methionine (Met); C—Cysteine (Cys); F—Phenylalanine (Phe); Y—Tyrosine (Tyr); W—Tryptophan (Trp); H—Histidine (His); K—Lysine (Lys); R—Arginine (Arg); Q—Glutamine (Gln); N—Asparagine (Asn); E—Glutamic Acid (Glu); D—Aspartic Acid (Asp); S—Serine (Ser); or T—Threonine (Thr).

As used herein, the terms “acidic” or “anionic” are used interchangeably to refer to amino acids such as aspartic acid and glutamic acid.

As used herein, the terms “basic” and “cationic” are used interchangeably to refer to amino acids such as arginine, histidine, and lysine.

The phrases “percent identity” or “sequence identity” as used herein refer to the number of amino acids in a sequence that are identical within a defined length of two peptide or protein segments in an alignment resulting in the maximal number of identical elements, and is calculated by dividing the number of identical elements by the total number of elements in the defined length of the aligned segments and multiplying by 100.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

The practice of the present disclosure will employ, unless indicated specifically to the contrary, conventional methodology and techniques that are in common use in the fields of microbiology, molecular biology, structural biology, and enzymology, which methodology and techniques are well known by and readily available to those having skill of the art. Such methodology and techniques are explained fully in laboratory manuals as well as the scientific and patent literature. See, e.g., Sambrook, et al., “Molecular Cloning: A Laboratory Manual” (2nd Edition, Cold Spring Harbor Laboratory Press, Cold Spring Harbor, N.Y., 1989); Maniatis et al., “Molecular Cloning: A Laboratory Manual” (1982); “DNA Cloning: A Practical Approach, vol. I& II” (Glover, ed.); “Oligonucleotide Synthesis” (Gait, ed., 1984); Ausubel et al. (eds.), “Current Protocols in Molecular Biology” (John Wiley & Sons, 1994); “Nucleic Acid Hybridization” (Hames & Higgins, eds., 1985); “Transcription and Translation” (Hames & Higgins, eds., 1984); “Animal Cell Culture” (Freshney, ed., 1986); and Perbal, “A Practical Guide to Molecular Cloning” (1984). All publications, patents and patent applications cited herein, whether supra or infra, are hereby incorporated by reference in their entirety.

### Antimicrobial Nodule-Specific Cysteine-Rich Peptide Folding Variant (NCR_PFV) Compositions

Within certain embodiments, the present disclosure provides compositions comprising a cationic antimicrobial nodule-specific cysteine-rich (NCR) peptide folding variant. The compositions according to these embodiments (1) include an NCR peptide folding variant 1 (NCR_PFV1) having a desired antimicrobial activity and/or resistance/sensitivity to proteolysis and (2) are substantially free of an NCR peptide folding variant 2 (NCR_PFV2) having decreased antimicrobial activity and/or resistance/sensitivity to proteolysis.

Exemplary cationic antimicrobial 6-Cys nodule-specific cysteine-rich peptides that can be used advantageously in the compositions disclosed herein are presented in Table 1 and include NCR peptides from the plants Cicer arietinum, Medicago truncatula, Galega orientalis, Medicago sativa, Astragalus canadensis, Pisum sativum, Ononis spinosa, Onobrychis viciifolia, and Oxytropis lambertii. Full length sequences for these NCR peptides are also provided in the sequence listing. Variants of such NCR peptides comprising amino acid additions (e.g., N- or C-terminal additions of one or more amino acid residues), insertions, deletions, or substitutions can also be used in the compositions provided herein.

TABLE 1
NODULE-SPECIFIC CYSTEINE-RICH (NCR) PEPTIDES
6-CYSTEINE

| | | | | | |
|---|---|---|---|---|---|
| | | Intervening | C3-5- | Intervening | |
| 6-Cys | C1-5AA-C2 | Sequence | 10AA-C4 | Sequence | C5-1AA-C6 |
| NCR Peptides | Motif | (1-8AA) | Motif | (4-6AA) | Motif |

Cicer arietinum

| | | | | | |
|---|---|---|---|---|---|
| Ca_NCR63 | C₁KTRVDC₂ | KKYR | C₃PRSKIKDC₄ | VKGY | C₅RC₆ |
| Ca_NCR62 | C₁KSHKDC₂ | KTHP | C₃PPSKVKQC₄ | VNGY | C₅RC₆ |
| Ca_NCR06 | C₁KSRVDC₂ | KTYP | C₃PIPKVKSC₄ | LNGY | C₅KC₆ |
| Ca_NCR61 | C₁KSWNDC₂ | KTYP | C₃PRPNGRNC₄ | VNGY | C₅RC₆ |
| Ca_NCR59 | C₁KSHNDC₂ | KTYP | C₃PTSKVKQC₄ | VNGY | C₅RC₆ |
| Ca_NCR11 | C₁TKDEDC₂ | PKDM | C₃KYPGSYPRC₄ | VWGW | C₅EC₆ |

Medicago truncatula

| | | | | | |
|---|---|---|---|---|---|
| Mt_NCR408 | C₁FRDSDC2 | PKVM | C3PSYLVTKC4 | FKKH | C5RC6 |
| Mt_NCR192 | C₁KHTGHC2 | PRKM | C3GAKTTKC4 | RNNK | C5QC6 |
| Mt_NCR667 | C₁KHTGHC2 | PRKM | C₃GAKTTKC₄ | RNNK | C₅QC₆ |
| Mt_NCR029 | C₁NSDDGC₂ | PNNY | C₃TPPHFGKC₄ | VSKQ | C₅VC₆ |
| Mt_NCR389 | C₁FKDSDC₂ | PKYM | C₃PSSLVAKC₄ | IKKL | C₅SC₆ |
| Mt_NCR569 | C₁HSDSAC₂ | ETTVK | C₃VLPRIPRC₄ | IKYK | C₅LC₆ |
| Mt_NCR703 | C₁FRDSNC₂ | PKSL | C₃HPPTKPKC₄ | MYKSI | C₅KC₆ |
| Mt_NCR492 | C₁FKDSEC₂ | LNLLY | C₃RTPLKPKC₄ | MYRTF | C₅KC₆ |
| Mt_NCR093 | C₁FKDSDC₂ | VKLL | C₃RIPLRPKC₄ | MYRHI | C₅KC₆ |
| Mt_NCR421 | C₁RVDFDC₂ | RMRM | C₃VYPTVSVC₄ | IDRL | C₅RC₆ |
| Mt_NCR407 | C₁FRDSDC₂ | PKNM | C₃PSYLVVKC₄ | LRSN | C₅KC₆ |
| Mt_NCR273 | C₁RNDSDC₂ | EKMVK | C₃VLPRIARC₄ | IKYR | C₅QC₆ |
| Mt_NCR388 | C₁RQNSDC₂ | PPYE | C₃IKPTVPKC₄ | IKFK | C₅LC₆ |
| Mt_NCR238 | C₁YNHTEC₂ | TNKIK | C₃VPPRIAQC₄ | FRFK | C₅DC₆ |
| Mt_NCR438 | C₁HNDSAC₂ | EKTVK | C₃MLPRIPRC₄ | IKYQ | C₅LC₆ |
| Mt_NCR098 | C₁LDHSHC₂ | PTEM | C₃SPGLKPKC₄ | MNPKV | C₅KC₆ |
| Mt_NCR384 | C₁SNDSAC₂ | QATTK | C₃VLPRVPRC₄ | IKYK | C₅LC₆ |
| Mt_NCR055 | C₁KDDFDC₂ | IENRLQVG | C₃RLQREKPRC₄ | VNLV | C₅RC₆ |
| Mt_NCR593 | C₁VFPSDC₂ | PRTMR | C₃LSEFHLTC₄ | KKKQ | C₅KC₆ |
| Mt_NCR189 | C₁FRDSDC₂ | PKHM | C₃PSSLVAKC₄ | INRL | C₅RC₆ |
| Mt_NCR317 | C₁KDHFEC₂ | RRKINILR | C₃IYRQEKPMC₄ | INSI | C₅TC₆ |
| Mt_NCR10189 | C₁NVDSDC₂ | PKNIWY | C₃ETGRINRC₄ | IRHQ | C₅RC₆ |
| Mt_NCR463 | C₁LYDKDC₂ | PQNM | C₃RPGRIPKC₄ | IFGH | C₅NC₆ |
| Mt_NCR420 | C₁VRNSDC₂ | RHHI | C₃MYPLVPRC₄ | KYPL | C₅RC₆ |
| Mt_NCR102 | C₁FHDADC₂ | VHKI | C₃HPPQIRKC₄ | VSKI | C₅KC₆ |
| Mt_NCR027 | C₁FRDSDC₂ | PKTL | C₃HSPGKAKC₄ | MHHSI | C₅KC₆ |
| Mt_NCR610 | C₁KNNFEC₂ | RFLI | C₃MNPLVPKC₄ | KVPY | C₅RC₆ |
| Mt_NCR666 | C₁KHDRNC₂ | PKNM | C₃LLTQIPKC₄ | FKNV | C₅KC₆ |
| Mt_NCR417 | C₁QTNYDC₂ | LRMW | C₃PIGISPRC₄ | IKRR | C₅KC₆ |
| Mt_NCR133 | C₁RNVYDC₂ | PKATY | C₃RAGSHRVQC₄ | IKHQ | C₅KC₆ |
| Mt_NCR308 | C₁RAHSHC₂ | VARIN | C₃VLPRKPQC₄ | RNYA | C₅GC₆ |
| Mt_NCR212 | C₁FRDSDC₂ | PKEM | C₃NHPKIPKC₄ | VNNAY | C₅KC₆ |
| Mt_NCR510 | C₁REDSHC₂ | VTKVK | C₃GLPRTPKC₄ | RNYI | C₅FC₆ |

Galega orientalis

| | | | | | |
|---|---|---|---|---|---|
| Go_NCR280 | C₁YADSDC₂ | LKTM | C₃DQRPWNQWC₄ | INNK | CsVC₆ |
| Go_NCR274 | C₁KTKRQC₂ | PS | C₃FHPHKVDC₄ | VKGF | C₅RC₆ |
| Go_NCR269 | C₁HHHKDC₂ | ANHK | C₃TIRHRVPKC₄ | LFYW | C₅AC₆ |
| Go_NCR252 | C₁KHKRDC₂ | TI | C₃YPPLKPDC₄ | IKRK | C₅HC₆ |
| Go_NCR253 | C₁RNKRDC₂ | PT | C₃YHPKKRDC₄ | IKHE | C₅GC₆ |
| Go_NCR257 | C₁RMNSEC₂ | PKKM | C₃TPFMIRKC₄ | IWNR | CSEC₆ |
| Go_NCR250 | C₁KSYLDC₂ | MDF | C₃ILPQYRRC₄ | IWDK | CSIC₆ |
| Go_NCR254 | C₁SFTWDC₂ | YYL | C₃ PVGKVAVC₄ | NWGR | C₅TC₆ |
| Go_NCR244 | C₁KKYTDC₂ | REML | C₃LPLQVRQC₄ | KNSY | C₅QC₆ |
| Ms_NCR445 | C₁FRDSDC₂ | PKHM | C₃PSNMVAKC₄ | IHRL | C₅KC₆ |
| Ms_NCR441 | C₁KENYGC₂ | PIFF | C₃ATWIPKC₄ | IKNK | C₅YC₆ |
| Ms_NCR435 | C₁KAAIDC₂ | GKNE | C₃IRPLKAKC₄ | IMFT | C₅FC₆ |
| Ms_NCR453 | C₁RVDFDC₂ | RMRM | C₃VYPTVPVC₄ | IDRL | CsRC₆ |
| Ms_NCR454 | C₁RVDFDC₂ | RMRM | C₃VYPTVPVC₄ | IDRL | C₅RC₆ |
| Ms_NCR432 | C₁VHVSKC₂ | PKMY | C₃NRGYELSC₄ | VQKY | CsQC₆ |
| Ms_NCR407 | C₁HDVSSC₂ | PKKMY | C₃KVGTHMQC₄ | VKHQ | C₅KC₆ |
| Ms_NCR415 | C₁FRDSDC₂ | PKTL | C₃HS PGKAKC₄ | MHHSI | C₅KC₆ |
| Ms_NCR408 | C₁KKNADC₂ | PKYL | C₃MLPLKPKC₄ | NETFW | CsTC₆ |
| Ms_NCR414 | C₁QTIVDC₂ | RYKA | C₃YRPLKPQC₄ | IDGK | CsVC₆ |
| Ms_NCR416 | C₁NLDDGC₂ | PNNY | C₃TPPHVGKC₄ | VSKH | CsVC₆ |

Astragalus canadensis

| | | | | | |
|---|---|---|---|---|---|
| Ac_NCR108 | C₁IRNSDC₂ | PKNM | C₃PLGKTPAC₄ | TKRR | CsRC₆ |
| Ac_NCR104 | C₁TNDTQC₂ | APLY | C₃LEEGTKSRC₄ | MKGR | CsRC₆ |
| Ac_NCR105 | C₁TNDTQC₂ | APLY | C₃LEEGTKSRC₄ | MKGR | CsRC₆ |
| Ac_NCR106 | C₁KQDFDC₂ | QQLR | C₃LSGYKPVC₄ | HRRRV | C₅KC₆ |
| Ac_NCR107 | C₁FTAGDC₂ | PVDE | C₃ENKVGSKIVC₄ | SFLYV | CsRC₆ |
| Ac_NCR099 | C₁FQDSDC₂ | PPDM | C₃LPSLTPKC₄ | IMGY | CsVC₆ |
| Ac_NCR103 | C₁IRGSNC₂ | PRNM | C₃PSGSRTKC₄ | IRHV | CSEC₆ |
| Ac_NCR098 | C₁KRGVDC₂ | PKNM | C₃PPWSKPMC₄ | ERRI | C₅KC₆ |
| Ac_NCR100 | C₁RQDSDC₂ | TKNM | C₃RSGFKPAC₄ | IRRV | C₅KC₆ |
| Ac_NCR097 | C₁KQDSDC₂ | QQLR | C₃LSGYKPVC₄ | SSWRV | C₅KC₆ |
| Ac_NCR096 | C₁RYHFHC₂ | KNMY | C₃FQEPKC₄ | VLGI | CSEC₆ |

Pisum sativum

| | | | | | |
|---|---|---|---|---|---|
| Ps_NCR349 | C₁TNDKDC₂ | PTMM | C₃FPPDVSKC₄ | VWKT | C₅YC₆ |
| Ps_NCR347 | C₁VRDGDC₂ | FTHL | C₃QPPRMVKC₄ | DNKK | C₅TC₆ |
| Ps_NCR348 | C₁RTDSDC₂ | PKQM | C₃RFPTYNKC₄ | VIRNH | C₅QC₆ |
| Ps_NCR346 | C₁LEHSHC₂ | YRVL | C₃RRPAIPKC₄ | DFRRGL | C₅KC₆ |
| Ps_NCR343 | C₁SSDSDC₂ | RLNK | C₃ERPRISKC₄ | VRNW | CsRC₆ |
| Ps_NCR344 | C₁SSDSDC₂ | RLNK | C₃ERPRISKC₄ | VRNW | CsRC₆ |
| Ps_NCR338 | C₁FKDSDC₂ | PKDW | C₃RKPKIMTC₄ | VSHSL | C₅KC₆ |
| Ps_NCR340 | C₁SRDSDC₂ | RINML | C₃RKPKIAKC₄ | VARM | CSNC₆ |
| Ps_NCR331 | C₁VHDREC₂ | K | C₃PGHPVSRC₄ | VKGF | CsRC₆ |
| Ps_NCR339 | C₁FKDSHC₂ | PDS | C₃RRPKIMKC₄ | VFQSY | C₅KC₆ |
| Ps_NCR335 | C₁FDDKDC₂ | VKRLY | C₃RIPTKAKC₄ | VSIF | C₅KC₆ |
| Ps_NCR329 | C₁SKDLDC₂ | PKSM | C₃KSPMRVKC₄ | WYSSF | CRC₆ |
| Ps_NCR336 | C₁QTKTDC₂ | RDKA | C₃YRPLKRKC₄ | ISYR | CSEC₆ |
| Ps_NCR337 | C₁EKDIHC₂ | PRSS | C₃PPYLYAKC₄ | VRER | C₅YC₆ |
| Ps_NCR326 | C₁IRTKDC₂ | PKSW | C₃YPPQKPKC₄ | IRSK | CDC₆ |
| Ps_NCR321 | C₁LTDSDC₂ | NKNK | C₃RKPTIIKC₄ | VDRL | C₅TC₆ |
| Ps_NCR323 | C₁FGDSDC₂ | KNRMF | C₃RSPFKVTC₄ | INNNH | C₅KC₆ |
| Ps_NCR328 | C₁VTVDDC₂ | SRRV | C₃GFGLKRIC₄ | YRDE | CsRC₆ |
| Ps_NCR333 | C₁FRDSDC₂ | PPNL | C₃QGPYVAKC₄ | VGRR | CsRC₆ |
| Ps_NCR308 | C₁QTDVDC₂ | RNRA | C₃HRPLKRKC₄ | VFFY | CSEC₆ |
| Ps_NCR324 | C₁RNDSDC₂ | ENRA | C₃QPPRIVKC₄ | DNKQ | C₅AC₆ |
| Ps_NCR309 | C₁SGDHDC₂ | PKNW | C₃PLSMKHKC₄ | KSGY | C₅KC₆ |
| Ps_NCR315 | C₁YKVEHC₂ | DSSI | C₃RSPYVLKC₄ | RMER | CSIC₆ |
| Ps_NCR307 | C₁NDDHDC₂ | PKKW | C₃PFSMKHKC₄ | KGGY | C₅KC₆ |
| Ps_NCR314 | C₁FDDSDC₂ | ENKIY | C₃RPPYRGKC₄ | MNRTN | C₅KC₆ |
| Ps_NCR318 | C₁HNNHGC₂ | TRYLS | C₃PPPLVAKC₄ | YNFY | C₅KC₆ |
| Ps_NCR306 | C₁IHDRQC₂ | R | C₃TQHTVSKC₄ | INGF | C₅KC₆ |
| Ps_NCR310 | C₁YEDKDC₂ | LQMR | C₃IVPNSPTC₄ | VGSV | C₅RC₆ |
| Ps_NCR312 | C₁KEHSDC₂ | ANYE | C₃VTPYVPKC₄ | LYLK | CsRC₆ |
| Ps_NCR313 | C₁KKRYDC₂ | PRDM | C₃IPRITPKC₄ | MNGI | C₅MC₆ |

Ononis spinosa

| | | | | | |
|---|---|---|---|---|---|
| Os_NCR228 | C₁VIHDHC₂ | PKEI | C₃SEDRTPKC₄ | YDGI | CSYC₆ |
| Os_NCR230 | C₁YENKNC₂ | PQKL | C₃PYNLIAAC₄ | RSHV | C₅KC₆ |
| Os_NCR229 | C₁IRDGDC₂ | PRRV | C₃IFPFKVRC₄ | VRTN | C₅KC₆ |
| Os_NCR221 | C₁YSVNDC₂ | MERLPFP | C₃ERGRSDQLVKC₄ | QNMQ | CsVC₆ |
| Os_NCR220 | C₁VKDSDC₂ | RKVM | C₃PVGSRPRC₄ | YFNY | C₅KC₆ |
| Os_NCR222 | C₁RDSRNC₂ | PPFF | C₃KY PKVAKC₄ | IQFK | C₅TC₆ |
| Os_NCR225 | C₁TNNKEC₂ | KGYYR | C₃RSRESPVC₄ | FMGL | CsDC₆ |
| Os_NCR219 | C₁VIHDHC₂ | PKEI | C₃SDDRTPKC₄ | YDGI | C₅YC₆ |

Onobrychis viciifolia

| | | | | | |
|---|---|---|---|---|---|
| Ov_NCR171 | C₁IKASDC₂ | PKKM | C₃PRSLKRKC₄ | IERK | CSIC₆ |
| Ov_NCR170 | C₁FKAWNC₂ | PKKM | C₃PNGVIPWC₄ | MKRK | C₅AC₆ |
| Ov_NCR169 | C₁KTATNC₂ | PLAM | C₃TAPHSRWC₄ | YKRR | CsRC₆ |
| Ov_NCR165 | C₁VVHNDC₂ | KHSGGF | C₃TYFRKC₄ | VRGE | CsRC₆ |
| Ov_NCR166 | C₁KMDSHC₂ | PGP | C₃PYLHYPSC₄ | RNHY | CsVC₆ |

Oxytropis lambertii

| | | | | | |
|---|---|---|---|---|---|
| O1_NCR36 | C₁RNNKQC₂ | APLI | C₃VDRKPWC₄ | IKGG | C₅RC₆ |
| O1_NCR35 | C₁RWESDC₂ | PKNM | C₃FTGFTPRC₄ | IKFV | CsTC₆ |

Within certain aspects of these compositions, the cationic antimicrobial nodule-specific cysteine-rich (NCR) peptide is a 6-Cys NCR peptide having (1) a first cysteine pair motif including C₁ and C₂, (2) a first intervening sequence (IS1), (3) a second cysteine pair motif including C₃ and C₄, (4) a second intervening sequence (IS2), and (5) a third cysteine pair motif including C₅ and C₆. Typically, cationic 6-Cys NCR according to these embodiments have an isoelectric point of between 8.0 and 12.0 and/or the NCR_PFV1 exhibits greater protease resistance than the NCR_PFV2. The aforementioned and after mentioned antimicrobial 6-Cys nodule-specific cysteine rich (NCR) peptides which are the object of this disclosure are peptides which have less than less than 60%, 70%, 80%, 85%, 90%, 92%, 95%, 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4).

According to these aspects, the cysteine residues C₁, C₂, C₃, C₄, C₅, and C₆ of the 6-Cys NCR peptide folding variant 1 (NCR_PFV1) form a first set of disulfide bonds to thereby adopt a first conformation that exhibits greater antimicrobial activity and preferred resistance/sensitivity to proteolysis as compared to an NCR peptide folding variant 2 (NCR_PFV2) having the same amino acid sequence as NCR_PFV1 but forming a distinct second set of disulfide bonds between cysteine residues C₁, C₂, C₃, C₄, C₅, and C₆ and adopting a distinct second conformation having an undesired reduction in antimicrobial activity and/or an undesired resistance/sensitivity to proteolysis.

Exemplified herein are 6-Cys NCR peptide folding variant 1 (NCR_PFV1) that form a the first set of disulfide bonds that comprise a C₁-C₄ disulfide bond, a C₂-C₅ disulfide bond, and a C₃-C₆ disulfide bond, wherein 6-Cys NCR peptide folding variant 2 (NCR_PFV1) forms a second set of disulfide bonds other than a C₁-C₄ disulfide bond and a C₂-C₅ disulfide bond such as, for example, a second set of disulfide bonds that comprise a C₁-C₂ disulfide bond, a C₃-C₆ disulfide bond, and a C₄-C₅ disulfide bond.

In certain aspects of these embodiments, 6-Cys NCR can include a first cysteine pair motif that is a C₁-5AA-C₂ motif having in contiguous sequence from its N-terminal end five amino acids (A₁, A₂, A₃, A₄, and A₅) flanked by an N-terminal C₁ and a C-terminal C₂ wherein A₅ can be an anionic amino acid selected from Asp (D) and Glu (E) or a cationic amino acid selected from His (H), Arg (R), and Lys (K) or an aliphatic amino acid selected from Gly (G), Ala (A), Val (V), Leu (L), Ile (I), and Pro (P); wherein A₄ can be a cationic amino acid selected from His (H), Arg (R), and Lys (K) or an aliphatic amino acid selected Gly (G), Ala (A), Val (V), Leu (L), Ile (I), and Pro (P) or an amide amino acid selected from Asn (N) and Gln (Q) or a polar amino acid selected from Ser (S) and Thr (T); and wherein A₃ can be a cationic amino acid selected from His (H), Arg (R), and Lys (K) or an anionic amino acid selected from Asp (D) and Glu (E) or a polar amino acid selected from Ser (S) and Thr (T).

In other aspects of these embodiments, 6-Cys NCR can include a second cysteine pair motif is a C₃-5-10AA-C₄ motif that comprises in contiguous sequence from its N-terminal end from five to ten amino acids (A₆, A₇, A₈, A₉, A₁₀ up through A₁₁, A₁₂, A₁₃, A₁₄, A₁₅) flanked by an N-terminal C₃ and a C-terminal C₄. Exemplified herein are 6-Cys NCR wherein the C₃-5-10AA-C₄ motif comprises in contiguous sequence from its N-terminal end seven amino acids (A₆, A₇, A₈, A₉, A₁₀, A₁₁, and A₁₂) flanked by an N-terminal C₃ and a C-terminal C₄ wherein the A₆ is an aliphatic amino acid selected from Gly (G), Ala (A), Val (V), Leu (L), Ile (I), and Pro (P) or a cationic amino acid selected from His (H), Arg (R), and Lys (K); wherein A₁₂ is an anionic amino acid selected from Asp (D) and Glu (E) or a cationic amino acid selected from His (H), Arg (R), and Lys (K) or an amide amino acid selected from Asn (N) and Gln (Q).

In further aspects of these embodiments, 6-Cys NCR can include a C₅-1AA-C₆ motif that comprises one amino acid (A₁₆) flanked by an N-terminal C₅ and a C-terminal C₆ wherein the A₁₆ is a cationic amino acid selected from His (H), Arg (R), and Lys (K) or an aliphatic amino acid selected from Gly (G), Ala (A), Val (V), Leu (L), Ile (I), and Pro (P) or an anionic amino acid selected from the group consisting of Asp (D) and Glu (E).

In related aspects of these embodiments, 6-Cys NCR can include a first intervening sequence (IS1) having from one to eight amino acids or from four to five amino acids and a second intervening sequence (IS2) having from four to six amino acids.

Representative 6-Cys NCR according to these embodiments can comprise an amino acid sequence having at least 60%, 70%, 75%, 80%, 85%, 90%, 95%, 97%, or 99% sequence identity to any one of NCRs presented in Table 1 or the corresponding NCRs in the sequence listing.

Depending upon the precise application contemplated, compositions according to these embodiments can comprise an agriculturally, pharmaceutically, or veterinary practicable carrier, diluent, or excipient.

Exemplary cationic antimicrobial 4-Cys nodule-specific cysteine-rich peptides that can be used advantageously in the compositions disclosed herein are presented in Table 2 and include NCR peptides from the plants Medicago truncatula, Galega orientalis, M. sativa, Astragalus canadensis, Pisum sativum, and Ononis spinosa. Full length sequences for these NCR peptides are also provided in the sequence listing. Variants of such NCR peptides comprising amino acid additions (e.g., N- or C-terminal additions of one or more amino acids), insertions, deletions, or substitutions can also be used in the compositions provided herein.

TABLE 2
4-CYSTEINE NODULE-SPECIFIC CYSTEINE-RICH (NCR) PEPTIDES
from Medicago truncatula

| | | | | Carboxy |
|---|---|---|---|---|
| 4-Cys | | Intervening | | terminal |
| NCR | C₁-5AA-C₂ | Sequence | C₃-4AA-C₄ | sequence |
| Peptides | Motif | (8-13AA) | Motif | (3-6AA) |
| Mt_NCR044 | C₁ISDKEC₂ | SIVKNYRAR | C₃RKGYC₄ | VRRRIR |
| Mt_NCR335 | C₁KTNKDC₂ | PKLRRANVR | C₃RKSYC₄ | VPI |
| Mt_NCR030 | C₁ITNKDC₂ | RQVRNYIAR | C₃RKGQC₄ | LQSPVR |
| Mt_NCR485 | C₁ITDKDC₂ | LQVRNYIAR | C₃RKGIC₄ | QQHPRR |
| Mt_NCR183 | C₁KTDKDC₂ | KQHRGENFR | C₃RSGNC₄ | IPIRR |
| Mt_NCR473 | C₁ITDKDC₂ | SPMKNYKAR | C₃RKGYC₄ | VSRRIR |
| Mt_NCR0181 | C₁QTAADC₂ | PEGTRGRTYK | C₃INNKC₄ | RYPKLL |
| Mt_NCR137 | C₁LTDKDC₂ | PRMPPHNIK | C₃RKGHC₄ | VPIGKP |
| Mt_NCR722 | C₁VTDKDC₂ | PKVAKINIR | C₃RRGQC₄ | VQVF |
| Mt_NCR723 | C₁VTDKDC₂ | PKVAKINIR | C₃RRGQC₄ | VQVE |
| Mt_NCR184 | C₁KTDKDC₂ | QNHRGENER | C₃RKGNC₄ | VAKIR |
| Mt_NCR032 | C₁KTDKDC₂ | KPKRGVNFR | C₃RKGKC₄ | YPR |
| Mt_NCR144 | C₁ITDKDC₂ | PNMKHYKAK | C₃RKGFC₄ | ISSRVR |
| Mt_NCR371 | C₁KTDKDC₂ | PKLGRANIR | C₃REGYC₄ | VRI |
| Mt_NCR207 | C₁KTVHDC₂ | PKSQVVYR | C₃VGNYC₄ | RAVKIR |
| Mt_NCR654 | C₁KSTNDC₂ | PKSVLRVWR | C₃INNYC₄ | RPVRMK |
| Mt_NCR603 | C₁IRDKDC₂ | LKFYGANIR | C₃RKGKC₄ | VAI |
| Mt_NCR020 | C₁TTDKDC₂ | KQVKGYIAR | C₃RKGYC₄ | MQSVKR |
| Mt_NCR358 | C₁ITDKDC₂ | PQMKINNVR | C₃RKGFC₄ | IQIHKF |
| Mt_NCR471 | C₁ISDKDC₂ | PQVKNFSAR | C₃RKGYC₄ | QFRVVR |
| Mt_NCR533 | C₁TTDKDC₂ | SKVEYGYKLR | C₃RKGRC₄ | VHIPR |
| Mt_NCR155 | C₁VTDKDC₂ | PRKNRHVVK | C₃RKGYC₄ | VGVQII |
| Mt_NCR609 | C₁ITDKDC₂ | PKHRGVNGK | C₃RKGYC₄ | VGVGRN |
| Mt_NCR00224 | C₁KSLNDC₂ | PKAIKPIFVK | C₃LGNIC₄ | QYSIGR |
| Mt_NCR042 | C₁LRDKDC₂ | PQLKFHNIR | C₃RNGFC₄ | QASKLR |
| Mt_NCR181 | C₁ITDKDC₂ | PSVKNYIGR | C₃RKGYC₄ | RYPKLL |
| Mt_NCR | C₁QTAADC₂ | PEGTRGRTYK | C₃INNKC₄ | RYPKLL |
| Mt_NCR619 | C₁KTVKDC₂ | PIRRNRKYY | C₃LFGIC₄ | VKL |
| Mt_NCR617 | C₁KTDKDC₂ | PQFRRANIR | C₃RKGQC₄ | VPI |
| Mt_NCR101 | C₁KTDKDC₂ | PKFGRVNVR | C₃RKGNC₄ | VKFMMK |
| Mt_NCR436 | C₁ENAASC₂ | PRLMYPLVYK | C₃LDNKC₄ | RYTIPY |
| Mt_NCR046 | C₁ITFEDC₂ | PISKTRVYK | C₃LHGEC₄ | VTV |
| Mt_NCR611 | C₁KTDKDC₂ | PKLRGVNVR | C₃RKDQC₄ | VLE |
| Mt_NCR474 | C₁KIDKDC₂ | PKLRGGSFR | C₃RKGKC₄ | QQSPLR |
| Mt_NCR047 | C₁ITDKDC₂ | PNGRNYIGR | C₃RKGQC₄ | LLIPRG |
| Mt_NCR511 | C₁KTDKDC₂ | PQLRGYTAR | C₃RKTQC₄ | YKLNR |
| Mt_NCR250 | C₁RTDQDC₂ | PRKKKESVT | C₃RKGFC₄ | FYGFGK |
| Mt_NCR035 | C₁VTVADC₂ | PPVKKPLKMW | C₃IRQTC₄ | VPPI |
| Mt_NCR515 | C₁KRDKDC₂ | PKLYGANFR | C₃RKGTC₄ | VRI |
| Mt_NCR614 | C₁KTDKDC₂ | PKYQRANIR | C₃RKGQC₄ | VQI |
| Mt_NCR702 | C₁EKDKDC₂ | PKPPRINIRINIR | C₃RKGFC₄ | AQM |
| Mt_NCR170 | C₁RTDKDC₂ | PKKMGTVGK | C₃RKGYC₄ | VQLG |
| Mt_NCR148 | C₁KTDKDC₂ | PKFYGSNVR | C₃RKGKC₄ | VEIL |
| Mt_NCR490 | C₁KTDKDC₂ | PKKPPHNIR | C₃RKGQC₄ | VHI |
| Mt_NCR700 | C₁TTDKDC₂ | PKVQHGYKLR | C₃RKGQC₄ | VNLILR |
| Mt_NCR483 | C₁ITDKDC₂ | IRRAGMNIR | C₃RKGYC₄ | VDGLIG |
| Mt_NCR | C₁ITDKDC₂ | PALKKNKGKGR | C₃RKGYC₄ | VDGLIG |
| Mt_NCR143 | C₁ITDKDC₂ | PSWKNYTGR | C₃RKGFC₄ | ISGRLR |
| Mt_NCR377 | C₁TTVKDC₂ | PEVKNYKSR | C₃LKGLC₄ | VQVNGG |
| Mt_NCR300 | C₁VTDKDC₂ | PKFKKYNIR | C₃RKGFC₄ | RLDM |
| Mt_NCR021 | C₁TQTSDC₂ | YKKYPFIPWGKVR | C₃VKGRC₄ | VKIKI |
| Mt_NCR583 | C₁KSDKDC₂ | KDIIIYRYILK | C₃RNGEC₄ | VSYSII |
| Mt_NCR537 | C₁ITNNDC₂ | PPRTRLILYK | C₃RNRKC₄ | RYREAV |
| Mt_NCR613 | C₁ETVADC₂ | PESYFRIYR | C₃ENNFC₄ | VRI |
| Mt_NCR119 | C₁ITDKDC₂ | SRVKNYNAR | C₃RKGYC₄ | TPIDS |
| Mt_NCR043 | C₁KRDKDC₂ | PQFRGVNIR | C₃RSGFC₄ | VQI |
| Mt_NCR | C₁KSDKDC₂ | PKLHRSNVR | C₃RKGQC₄ | VQI |
| Mt_NCR | C₁ISDKDC₂ | QKVHGVNIR | C₃RKGFC₄ | TVI |
| Mt_NCR199 | C₁KTDKDC₂ | PKLHKVNVR | C₃RKGKC₄ | QQRLVR |
| Mt_NCR474 | C₁ITDKDC₂ | PNGRNYIGR | C₃RKGHC₄ | VNLGN |
| Mt_NCR034 | C₁VTDKDC₂ | PRPQFRKSNVR | C₃RNGYC₄ | VNLGN |
| Mt_NCR052 | C₁STDDHC₂ | KQVSSKFYRHI | C₃HNNRC₄ | VQV |
| Mt_NCR649 | C₁KTDKDC₂ | PKVQGANIR | C₃RSGKC₄ | GKRTLY |
| Mt_NCR038 | C₁LTDKDC₂ | TRRKGFSVT | C₃RKGFC₄ | VEFKHF |
| Mt_NCR | C₁IKDKDC₂ | PKVKSENIR | C₃RKGFC₄ | VNIL |
| Mt_NCR632 | C₁ITDKDC₂ | PSLEKNKGKGR | C₃RKGYC₄ | VHGLIG |
| Mt_NCR718 | C₁VTVKDC₂ | PKTVFPIGYK | C₃IKNLC₄ | ILVI |
| Mt_NCR472 | C₁ITDKDC₂ | PNVQNYIGR | C₃RKGFC₄ | ISSWVR |
| Mt_NCR521 | C₁KIDKDC₂ | PRNPPLNIR | C₃RKSFC₄ | VQI |
| Mt_NCR | C₁VSDKDC₂ | PRPKFQKSNVR | C₃RKGYC₄ | VNLDG |
| Mt_NCR | C₁LTDKDC₂ | PKKNGYVGK | C₃RKGYC₄ | VGI |
| Mt_NCR | C₁VIDKDC₂ | PQFKNNNVR | C₃RKGFC₄ | VNLSVN |
| Mt_NCR520 | C₁ETVADC₂ | PTHWAYIYV | C₃EKNKC₄ | RYHEKS |
| Mt_NCR303 | C₁IYDHDC₂ | PRKLYFLER | C₃VGRVC₄ | KYL |
| Mt_NCR361 | C₁VTDKDC₂ | PKVSQYNIR | C₃RKGQC₄ | ARIRV |
| Mt_NCR048 | C₁MTDNDC₂ | APEKYYNIR | C₃RKGFC₄ | VQIRKY |
| Mt_NCR246b | C₁VTELDC₂ | YKKYRLPAEKKMK | C₃IRGSC₄ | YRVAHF |
| Mt_NCR452 | C₁ASTNEC₂ | YTTFKFAPLGSMR | C₃VEGYC₄ | KHLKDE |

Within certain aspects of these compositions, the cationic antimicrobial nodule-specific cysteine-rich (NCR) peptide is a 4-Cys NCR peptide having (1) a first cysteine pair motif including C₁ and C₂, (2) a first intervening sequence (IS1), and (3) a second cysteine pair motif including C₃ and C₄. Typically, cationic 4-Cys NCR according to these embodiments have an isoelectric point of between 8.0 and 12.0 and/or the NCR_PFV1 exhibits greater protease resistance than the NCR_PFV2. The aforementioned and after mentioned antimicrobial 4-Cys nodule-specific cysteine rich (NCR) peptides which are the object of this disclosure are peptides which have less than 60% sequence identity to CaNCR7 (SEQ ID NO: 2), CaNCR13 (SEQ ID NO: 1), CaNCR14 (SEQ ID NO: 3), or CaNCR15 (SEQ ID NO: 4).

According to these embodiments, the cysteine residues C₁, C₂, C₃, and C₄ of the 4-Cys NCR peptide folding variant 1 (NCR_PFV1) form a first set of disulfide bonds to thereby adopt a first conformation that exhibits greater antimicrobial activity and preferred resistance/sensitivity to proteolysis as compared to an NCR peptide folding variant 2 (NCR_PFV2) having the same amino acid sequence as NCR_PFV1 but forming a distinct second set of disulfide bonds between cysteine residues C₁, C₂, C₃, and C₄ and adopting a distinct second conformation having decreased antimicrobial activity and/or undesired resistance or undesired sensitivity to proteolysis.

Exemplified herein are 4-Cys NCR peptide folding variant 1 (NCR_PFV1) that form a first set of disulfide bonds that comprise a C₁-C₂ disulfide bond and a C₃-C₄ disulfide bond, wherein 4-Cys peptide folding variant 2 (NCR_PFV2) forms a second set of disulfide bonds other than a C₁-C₂ disulfide bond and a C₃-C₄ disulfide bond such as, for example, a second set of disulfide bonds that comprise a C₁-C₃ disulfide bond and a C₂-C₄ disulfide bond or a second set of disulfide bonds that comprise a C₁-C₄ disulfide bond and a C₂-C₃ disulfide bond.

In certain aspects of these embodiments, 4-Cys NCR can include a first cysteine pair motif that is a C₁-5AA-C₂ motif having in contiguous sequence from its N-terminal end five amino acids (A₁, A₂, A₃, A₄, and A₅) flanked by an N-terminal C₁ and a C-terminal C₂ wherein A₅ can be an anionic amino acid selected from Asp (D) and Glu (E); wherein A₄ can be a cationic amino acid selected from the group consisting of His (H), Arg (R), and Lys (K); and/or wherein A₃ can be an anionic amino acid selected from the group consisting of Asp (D) and Glu (E).

In other aspects of these embodiments, 4-Cys NCR can include a second cysteine pair motif that is a C₃-4AA-C₄ motif that comprises in contiguous sequence from its N-terminal end four amino acids (A₆, A₇, A₈, and A₉) flanked by an N-terminal C₃ and a C-terminal C₄ wherein A₆ is a cationic amino acid selected from His (H), Arg (R), and Lys (K) or an aliphatic amino acid selected from Gly (G), Ala (A), Val (V), Leu (L), Ile (I), and Pro (P); wherein A₇ is a cationic amino acid selected from His (H), Arg (R), and Lys (K) or an amide amino acid selected from Asn (N) and Gln (Q) or an aliphatic amino acid selected from Gly (G), Ala (A), Val (V), Leu (L), Ile (I), and Pro (P); and/or wherein A₈ is an aliphatic amino acid selected from Gly (G), Ala (A), Val (V), Leu (L), Ile (I), and Pro (P) or an amide amino acid selected from Asn (N) and Gln (Q) or a cationic amino acid selected from His (H), Arg (R), and Lys (K).

In related aspects of these embodiments, 4-Cys NCR can include a first intervening sequence (IS1) having from five to thirteen amino acids or from five to eleven amino acids.

Representative 4-Cys NCR according to these embodiments can comprise an amino acid sequence having at least 60%, 70%, 75%, 80%, 85%, 90%, 95%, 97%, or 99% sequence identity to any one of NCRs presented in Table 2 (other than, in certain embodiments, M. truncatula NCR169 (SEQ ID NO: 6) or NCR247 (SEQ ID NO: 5)) or corresponding 4-Cys NCR peptides presented in the sequence listing.

Depending upon the precise application contemplated, compositions according to these embodiments can comprise an agriculturally, pharmaceutically, or veterinary practicable carrier, diluent, or excipient.

6-Cys and 4-Cys NCR_PFV1 can be separated from mixtures comprising the desired NCR_PFV1 and one or more undesired NCR_PFV2 to provide compositions that are enriched for the desired NCR_PFV1. Compositions comprising the desired NCR_PFV1 typically exhibit improved unit activity (e.g., units of anti-microbial activity per micrograms or micromoles of NCR protein) relative to compositions comprising a mixture of the desired NCR_PFV1 and one or more reduced activity NCR_PFV2. Depending upon the precise application contemplated for the 6-Cys and 4-Cys NCR_PFV1 compositions, it can be desirable to employ an NCR_PFV1 have an increased protease resistance or an increased protease sensitivity (e.g., to a serine protease including trypsin) in comparison to the undesired NCR_PFV2.

Compositions comprising a desired 6-Cys or 4-Cys NCR_PFV1 can be substantially free of an undesired NCR_PFV2. In certain embodiments, such compositions can comprise less than about 20%, 10%, 5%, 2%, 1%, or 0.5% by weight of an undesired 6-Cys or 4-Cys NCR_PFV2 and, respectively, at least about 80%, 90%, 95%, 98%, 99%, or 99.5% by weight of a desired 6-Cys or 4-Cys NCR_PFV1. In certain embodiments, the composition comprising the desired NCR_PFV1 which is substantially free of the undesired NCR_PFV2 can comprise less than about 20%, 10%, 5%, 2%, 1%, or 0.5% of the combined total of the desired NCR_PFV1 and undesired NCR_PFV2 by weight in the composition as the undesired NCR_PFV2. In certain embodiments, the composition comprising the desired NCR_PFV1 which is substantially free of the undesired NCR_PFV2 can comprise at least about 80%, 90%, 95%, 98%, 99%, or 99.5% of the total NCR peptide in the composition by weight (e.g., total NCR peptide in the form of the desired NCR_PFV and one or more undesired NCR_PFV1 and one or more undesired NCR_PFV2) as the desired NCR_PFV1. In certain embodiments, less than 20%, 10%, 5%, 2%, 1%, or 0.5% of the total NCR peptide in the composition by weight (e.g., total NCR peptide in the form of the desired NCR_PFV1 and one or more undesired NCR_PFV2) is the undesired NCR PFV2 and respectively at least about 80%, 90%, 95%, 98%, 99%, or 99.5% of the total NCR peptide in the composition by weight is the desired NCR_PFV1.

Without seeking to be limited by theory, it is contemplated that compositions comprising or enriched for a desired 6-Cys or 4-Cys NCR_PFV1 that exhibit an increased protease resistance relative to an undesired 6-Cys or 4-Cys NCR_PFV2 can be useful in certain of the methods disclosed herein where an extended shelf-life or half-life of the NCR_PFV1 is desirable. Alternatively, it is contemplated that compositions comprising or enriched for a desired 6-Cys or 4-Cys NCR_PFV1 that exhibit an increased protease sensitivity relative to an undesired 6-Cys or 4-Cys NCR_PFV2 can be useful in such methods where a decreased half-life of the NCR_PFV1 is desirable.

Suitable 6-Cys or 4-Cys NCR peptides for generating NCR_PFV1 peptides can include 6-Cys NCR peptides comprising an amino acid sequence as set forth in Table 1 or 4-Cys NCR peptides comprising an amino acid sequence as set forth in Table 2. Variants of these 6-Cys and 4-Cys NCR peptides are contemplated wherein, for example, one or more of the hydrophobic, basic, and/or acidic amino acid residue(s) is substituted with an alternative hydrophobic, basic, and/or acidic amino acid residues. In embodiments provided herein, the 6-Cys and 4-Cys NCR peptide variants comprise an amino acid sequence having at least 60%, 70%, 80%, 85%, 90%, 92%, 95%. 97%, 98%, 99%, or 100% sequence identity across the entire length of one or more NCR peptide(s) presented in Tables 1 and 2 and/or as set forth as SEQ ID NO: 8 to 371 but have less than 60% sequence identity to CaNCR7 (SEQ ID NO: 2), CaNCR13 (SEQ ID NO: 1), CaNCR14 (SEQ ID NO: 3), or CaNCR15 (SEQ ID NO: 4). Suitable 6-Cys or 4-Cys NCR peptides used in the compositions and methods provided herein do not include any of the 6-Cys NCR peptides or variants thereof having more than 60% sequence identity to CaNCR7 (SEQ ID NO: 2), CaNCR13 (SEQ ID NO: 1), CaNCR14 (SEQ ID NO: 3), or CaNCR15 (SEQ ID NO: 4), which are disclosed in US Patent Publication No. US 2022/0061333, which is incorporated herein by reference in its entirety.

Conserved cysteine residues in 6-Cys NCR peptides can be identified as C₁, C₂, C₃, C₄, C₅, and C₆ as depicted in Table 1 and conserved cysteine residues in 4-Cys NCR peptides can be identified as C₁, C₂, C₃, and C₄ as depicted in Table 2. For 6-Cys NCR peptides, the conserved cysteine residue closest to the amino terminus is C₁ and the cysteine residue closest to the carboxy terminus is C₆ as depicted in Table 1 while for 4-Cys NCR peptides, the conserved cysteine residue closest to the amino terminus is C₁ and the cysteine residue closest to the carboxy terminus is C₄ as depicted in Table 2.

Within certain aspects of these embodiments, 6-Cys or 4-Cys NCR peptides that can adopt alternative disulfide bonding pairs and that constitute NCR peptide folding variants can have a net positive charge at neutral pH. For example, certain NCR peptides have a net positive charge at neutral pH of at least +4, +5, +6, +7, +8, +9, or +10 to +12, +13, +14, or +15 or, alternatively, of at least +5, +6, +7, +8, +9, or +10. It will be understood that such net positive charges in 6-Cys or 4-Cys NCR peptides can be achieved by methods that include: (i) maintaining cationic (basic) amino acid residues found in the NCR peptides (including NCR peptides presented in Tables 1 and 2, respectively) or substituting such residues with another cationic amino acid residue; (ii) substituting anionic or polar amino acid residues found in NCR peptides (including NCR peptides presented in Tables 1 and 2, respectively) with a basic amino acid residue; or a combination of (i) and (ii). Such net positive charges in 6-Cys or 4-Cys NCR peptides can be achieved by preferentially selecting or substituting a cationic amino acid residue at variable positions in the NCR peptide that correspond to a variable position.

Within other aspects of these embodiments, 6-Cys or 4-Cys NCR peptides that can adopt alternative disulfide bonding pairs and that constitute NCR peptide folding variants can include a significant percentage of hydrophobic amino acid residues. For example, 6-Cys or 4-Cys NCR peptides can comprise at least about 25%, 26%, 28% 30%, 32%, 34%, or 36% to 37%, 38%, 40%, 42%, or 45% hydrophobic amino acid residues or at least about 25%, 26%, 28% 30%, 32%, 34%, 36%, 37%, or 38% hydrophobic amino acid residues. It will be understood that such percentages of hydrophobic amino acids in 6-Cys or 4-Cys NCR peptides can be achieved by methods that include: (i) maintaining hydrophobic amino acid residues found in 6-Cys or 4-Cys NCR peptides (including NCR peptides presented in Tables 1 and 2, respectively) or substituting such residues with another hydrophobic amino acid residue or neutral polar amino acid residue; (ii) substituting polar amino acid residues found in 6-Cys or 4-Cys NCR peptides (including NCR peptides presented in Tables 1 and 2, respectively) with a hydrophobic amino acid residue; (iii) substituting neutral polar amino acids for hydrophobic amino acids; or a combination of (i), (ii), and (iii)). Such percentages of hydrophobic amino acids in 6-Cys or 4-Cys NCR peptides can be achieved by preferentially selecting or substituting a hydrophobic amino acid residue at variable positions in the 6-Cys or 4-Cys NCR peptide that correspond to a variable position.

Within further aspects of these embodiments, 6-Cys or 4-Cys NCR peptides include 4, 5, or 6 of the conserved C₁, C₂, C₃, C₄, C₅, and C₆ cysteine residues set forth in Tables 1 and 2, respectively. 6-Cys NCR_PFV can comprise disulfide bonds corresponding to C₁ to C₄, C₂ to C₅, and C₃ to C₆ disulfide bonds in a 6-Cys NCR peptide presented in Table 1 while 4-Cys NCR_PFV can comprise disulfide bonds corresponding to C₁-C₂ and C₃-C₄ disulfide bonds in a 4-Cys NCR peptide presented in Table 2.

6-Cys NCR peptides can also comprise 4 of the conserved C₁, C₂, C₃, C₄, C₅, and C₆ cysteine residues. One or two of the cysteinyl residues in 6-Cys NCR peptide can, for example, be substituted with a distinct amino acid residue or deleted to provide an NCR peptide with only 4 of the 6 conserved C₁, C₂, C₃, C₄, C₅, and C₆ cysteine residues. Alternatively, one or two of the C₁, C₂, C₃, C₄, C₅, or C₆ cysteine residues in a 6-Cys NCR peptide can be substituted with another amino acid residue including a glycine, serine, threonine, cysteine, cystine, tyrosine, asparagine, or glutamine residue to provide an NCR peptide with only 4 of the 6 conserved C₁, C₂, C₃, C₄, C₅, and C₆ cysteine residues. For example, it can be desirable to substitute one or two of the conserved cysteine residues with a serine residue to provide an NCR peptide with only 4 of the 6 conserved C₁, C₂, C₃, C₄, C₅, and C₆ cysteine residues. Such NCR peptides having only 4 of the 6 conserved cysteine residues can comprise a deletion or a substitution of the C₁ and C₆ cysteine residues and will thus comprise the C₂, C₃, C₄, and C₅ cysteine residues. Alternatively, an NCR peptide with only 4 of the 6 conserved cysteine residues can comprise a deletion or a substitution of the C₂ or C₆ cysteine residues in an NCR peptide comprising only the C₂, C₃, C₄, C₅, and C₆ cysteine residues and thus comprise the C₃, C₄, C₅, and C₆ cysteine residues or the C₂, C₃, C₄, and C₅ cysteine residues. In certain embodiments, conserved cysteine residues corresponding to a C₁ and a C₄ cysteine residue are substituted with another amino acid residue (e.g. serine) to provide an NCR peptide with only 4 of the 6 conserved C₁, C₂, C₃, C₄, C₅, and C₆ cysteine residues. In certain embodiments, conserved cysteine residues corresponding to a C₂ and a C₅ cysteine residue are substituted with another amino acid residue (e.g., serine) to provide an NCR peptide with only 4 of the 6 conserved C₁, C₂, C₃, C₄, C₅, and C₆ cysteine residues.

Within yet further aspects of these embodiments, 6-Cys or 4-Cys NCR peptides comprise a core consensus sequence GX₁CKCVX₂R or a variant thereof comprising an insertion, deletion, and/or substitution of one or more amino acid residues. For example, X₁ can be F or Y and X₂ can be absent or V or X₁ can be F, Y, W, I, V, A, or M and/or X₂ is absent or F, Y, W, I, V, A, or M. Alternatively, an NCR peptide can also comprise of variant of the GX₁CKCVX₂R core consensus wherein one or both of the cationic residues (K and/or R) can be substituted with another cationic amino acid residue (K, R, or H). Such an NCR peptide can also comprise a variant of the core consensus GX₁CKCVX₂R wherein one or both of the conserved tyrosine or valine amino acid residues are substituted with a hydrophobic amino acid (F, W, I, A, or M) or with a tyrosine residue (Y).

Suitable NCR peptides for use in preparing compositions according to these embodiments can comprise an amino acid sequence that is at least 60%, 70%, 75%, 80%, 85%, 90%, 95%, 98%, 99%, or 100% identical to one or more of the 6-Cys NCR peptide sequences presented in Table 1 or one or more of the 4-Cys NCR peptide sequences presented in Table 2 wherein one or more of the hydrophobic, basic, and/or acidic amino acid residues are substituted with other hydrophobic, basic, and/or acidic amino acid residues, respectively.

One or more amino acids in any of the 6-Cys or 4-Cys NCR peptides presented in Table 1 and Table 2, respectively, or in a variant 6-Cys or 4-Cys NCR peptide can be substituted with other amino acid(s), the charge and polarity of which is similar to that of the original amino acid, i.e., a conservative amino acid substitution. Substitutes for an amino acid within the 6-Cys or 4-Cys NCR peptide sequence can be selected from other members of the class to which the originally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (anionic; negatively charged) amino acids such as aspartic acid and glutamic acid; (2) basic (cationic; positively charged) amino acids such as arginine, histidine, and lysine; (3) neutral polar amino acids such as glycine, serine, threonine, cysteine, cystine, tyrosine, asparagine, and glutamine; (4) neutral nonpolar (hydrophobic) amino acids such as alanine, leucine, isoleucine, valine, proline, phenylalanine, tryptophan, and methionine. Conservative amino acid changes within NCR peptide sequences can be made by substituting one amino acid within one of these groups with another amino acid within the same group.

Biologically functional equivalents of NCR peptides can have 10 or fewer conservative amino acid changes, seven or fewer conservative amino acid changes, or five, four, three, two, or one conservative amino acid changes. The encoding nucleotide sequence (e.g., gene, plasmid DNA, cDNA, or synthetic DNA) will thus have corresponding base substitutions, permitting it to encode biologically functional equivalent forms of the NCR peptides.

Certain semi-conservative substitutions in NCR peptides including: (i) the substitution of a neutral polar amino acid residue with a neutral nonpolar (hydrophobic) amino acid residue; or (ii) the substitution of a neutral nonpolar (hydrophobic) amino acid residue with a neutral polar amino acid residue are also provided. In particular, semi-conservative substitutions of a neutral polar tyrosine residue with a hydrophobic amino acid residue are provided. Semi-conservative substitutions of a hydrophobic amino acid residue with tyrosine residue are also provided. Biologically functional equivalents of NCR peptides can have 10 or fewer semi-conservative amino acid changes, seven or fewer semi-conservative amino acid changes, or five, four, three, two, or one semi-conservative amino acid changes.

Functional fragments of any of the 6-Cys or 4-Cys NCR disclosed herein can include amino terminal deletions, carboxy terminal deletions, internal deletions, or any combination thereof which leave intact the conserved cysteine residues of a 6-Cys or 4-Cys NCR or contain at least 4 or 5 of the six conserved C₁, C₂, C₃, C₄, C₅, and C₆ cysteine residues of a 6-Cys NCR. Functional fragment can contain at least one, two, three, four, five, six, or seven or more amino acid residue deletions from the amino terminus, the carboxy terminus, an internal region, or any combination thereof. For example, antimicrobial fragments of a 6-Cys or 4-Cys NCR peptide can comprise at least about 10, 14, 15, 18, or 20 to about 22, 24, 25, 26, 27, or 28 amino acid residues of the C-terminus of the NCR peptide. Chimeric NCR peptides comprising portions of any of the 6-Cys or 4-Cys NCR disclosed herein or comprising variants or fragments of those 6-Cys or 4-Cys NCR can also be used either alone or in the NCR proteins provided herein.

### Methods for Making Testing, and Using Antimicrobial NCR_PFV Compositions

Within related embodiments, the present disclosure provides methods for making, testing, and using antimicrobial NCR_PFV compositions comprising a cationic antimicrobial nodule-specific cysteine-rich (NCR) peptide folding variant (PFV), which compositions (1) include an NCR peptide folding variant 1 (NCR_PFV1) having a desired antimicrobial activity and/or resistance/sensitivity to proteolysis and (2) exclude an NCR peptide folding variant 2 (NCR_PFV2) having a decreased antimicrobial activity and/or an undesired resistance or sensitivity to proteolysis.

6-Cys or 4-Cys NCR_PFV1 can comprise an amino acid sequence at least 50%, 60%, 70%, 75%, 80%, 85%, 90%, 95%, 98%, 99%, or 100% identical to an amino acid sequence presented in Table 1 or Table 2, respectively, or a variant of the amino acid sequence an amino acid sequence presented in Table 1 or Table 2, wherein one or more of the hydrophobic, basic, and/or acidic amino acid residues are substituted with hydrophobic, basic, and/or acidic amino acid residues, respectively; a variant of the amino acid sequence presented in Table 1 or Table 2, wherein one or more neutral polar amino (e.g., tyrosine) acid residues is substituted with a hydrophobic amino acid residue; and/or wherein one or more hydrophobic amino acid residues is substituted with a neutral polar amino acid residue (e.g., tyrosine); functional fragments thereof, and chimeras thereof.

6-Cys or 4-Cys NCR peptides or proteins can synthesized de novo from an NCR peptide sequence disclosed herein or can be expressed from a nucleotide sequence encoding a 6-Cys or 4-Cys NCR peptide or protein. The sequence of the peptide or protein-encoding nucleotide sequence can be deduced from the NCR peptide sequence by reference to the genetic code. Computer programs such as “BackTranslate” (GCG™ Package, Acclerys, Inc. San Diego, CA) can be used to convert a peptide sequence to the corresponding nucleotide sequence that encodes the peptide.

Expression of 6-Cys and 4-Cys NCR peptides in yeast and filamentous fungi to produce NCR_PFV1 is specifically contemplated herein. The construction of expression vectors for production of heterologous proteins in various yeast genera is well established. In general, such expression vectors typically comprise a promoter that is operably linked to a sequence of interest which is operably linked to a polyadenylation or terminator region. Examples of yeast genera that have been used to successfully express heterologous genes include Candida, Kluveromyces, Hansuela, Pichia, Saccharomyces, Schizosaccharomyces, and Yarrowia. A general description of expression vectors and transformation systems for Saccharomyces is found in Kingsman et al (1985) Biotechnol Genet Eng Rev. 3:377-416. Expression vectors and transformation systems useful for yeasts other than Saccharomyces are described in Reiser et al (1990) Adv Biochem Eng Biotechnol.; 43:75-102. Other examples of fungal systems which can be adapted for use in expressing NCR peptides include filamentous fungal systems such as Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes and Trichoderma systems (e.g., U.S. Pat. Nos. 11,046,736 and 11,180,767, each incorporated herein by reference in its entirety). Other systems which can be adapted for use in expressing NCR peptides include Chrysosporium lucknowense systems (e.g., U.S. Pat. Nos. 8,871,493 and 9,175,296, each incorporated herein by reference in its entirety).

Expression of NCR peptides in bacterial cells including Escherichia sp. (e.g., E. coli) to produce NCR_PFV is also specifically contemplated herein. Systems for expressing proteins which comprise disulfide bonds can be adapted for expression of the NCR peptides in E. coli include those disclosed in US Patent Application US20200172915, which is incorporated herein by reference in its entirety, and in Berkmen, M. Protein Expr Purif 82(1):240-51 (2012). Other systems useful for expression of proteins which comprise disulfide bonds can be adapted for expression of the NCR peptides in E. coli include those disclosed in Kuddus, Biotechnol Prog 233:1520-1528 (2017); Kiedzierska, Protein Expr Purif 6:82-88 (2008); Chang, Amino Acids 47:579-587 (2015); Buchko, Protein Science 27:1611-1623 (2018); Marques, J Appl Microbiol 106:1640-1648 (2008); and Pazgier, Protein Expr Pur 49:1-8 (2006).

In general, the promoter and polyadenylation region are selected based on their operability in a given bacterial, yeast, or fungal host. For example, the AOX1 or AOX2 promoters of Pichia can be used in conjunction with the AOX1, AOX2, p40, or p76 polyadenylation sequences of Pichia to express a heterologous protein such as an NCR peptide. Both the AOX1 and AOX2 promoters are particularly useful in Pichia as both promoters provide for abundant expression of the linked heterologous gene when induced by addition of methanol to the growth medium. The use of these Pichia promoters and polyadenylation sequences is described in U.S. Pat. No. 4,855,231, which is expressly incorporated herein by reference in its entirety. Similarly, the Hansuela MOX, DHAS, or FMDH promoters can be used to express heterologous proteins such as NCR in Hansuela. The MOX, DHAS, or FMDH promoters are particularly useful in Hansuela as these promoters provide for abundant expression of the linked heterologous gene when induced by addition of methanol to the growth medium. The use of the MOX and DHAS promoters in Hansuela is described in U.S. Pat. No. 5,741,672, while the use of the FMDH promoter in Hansuela is described in U.S. Pat. No. 5,389,525, each of which is expressly incorporated herein by reference in its entirety. For Kluveromyces, a Lactase promoter and polyadenylation sequence can be used to express heterologous genes such as NCR. Expression of heterologous genes that are operably linked to the Lactase promoter and polyadenylation sequence is achieved by growing Kluveromyces in the presence of galactose. The use of the Lactase promoter and polyadenylation sequences in Kluveromyces is described in U.S. Pat. No. 6,602,682, which is expressly incorporated herein by reference in its entirety.

Yeast, bacterial, or fungal expression vectors that provide for secretion of heterologous proteins such as NCR into the growth medium by transformed yeast or fungi are also contemplated. Secretion of the mature NCR peptide is typically achieved by operable linkage of a signal peptide sequence or a signal peptide and propeptide sequence to the mature NCR protein- or peptide-encoding sequence. Examples of useful signal peptides for secretion of heterologous proteins in yeast include but are not limited to an alpha-factor signal peptide, an invertase signal peptide, and a PHOl signal peptide, all of which are derived from yeast. The alpha-factor signal peptide is typically derived from Saccharomyces, Kluveromyces, or Candida, while the PHOl signal peptide is derived from Pichia.

A particularly useful signal peptide sequence or signal peptide and propeptide sequence for secretion of proteins in yeast is derived from the S. cerevisiae alpha-factor, and is described in U.S. Pat. Nos. 4,546,082, 4,588,684, 4,870,008, and 5,602,034, each of which is incorporated herein by reference in its entirety. The S. cerevisiae alpha-factor signal peptide and propeptide sequence consist of amino acids 1-83 of the primary, unprocessed translation product of the S. cerevisiae alpha mating factor gene (GenBank Accession Number: P01149). In certain embodiments, the signal peptide sequence of the alpha-mating factor comprising amino acids 1 to about 19 to 23 of the alpha-mating factor proprotein can be directly linked to the N-terminus of the mature NCR protein to provide for secretion of mature NCR protein. In this case, the signal peptide is cleaved from the mature NCR protein in the course of the secretion process. Alternatively, the signal peptide and propeptide of the alpha mating factor can be operably linked to the mature NCR encoding sequence via a cleavage site sequence. This cleavage site sequence can comprise a variety of sequences that provide for proteolytic processing of the leader sequence and gene of interest.

In the native S. cerevisiae alpha mating factor gene the cleavage site sequence corresponds to amino acid residues 84-89 and is represented by the sequence Lys84-Arg85-Glu86-Ala87-Glu88-Ala 89 (SEQ ID NO: 7). The sequence Lys-Arg corresponds to a KEX2 protease recognition site while the Glu-Ala-Glu-Ala sequence corresponds to a duplicated dipeptidylaminopeptidase or STE13 recognition site. In certain embodiments, a DNA fragment encoding the 89 amino acid S. cerevisiae alpha factor signal, propeptide coding region, and entire native spacer coding region (i.e., the N-terminal 89 amino acid residues of the alpha mating factor precursor protein containing both the Lys-Arg KEX2 protease cleavage site at residues 84 and 85 as well as the Glu-Ala-Glu-Ala dipeptidylaminopeptidase or STE13 recognition site at residues 86-89) is operably linked to the sequence encoding the mature NCR protein.

When the N-terminal 89 amino acids of the alpha mating factor precursor protein are fused to the N-terminus of a heterologous protein such as NCR, the propeptide sequence is typically dissociated from the heterologous protein via the cleavage by endogenous yeast proteases at either the KEX2 or STE13 recognition sites. In other embodiments, a DNA fragment encoding the smaller 85 amino acid Saccharomyces cerevisiae alpha factor signal peptide, propeptide, and KEX2 spacer element (i.e., the N-terminal 85 amino acid residues of the alpha mating factor precursor protein containing just the Lys-Arg KEX2 protease cleavage site at residues 84 and 85) is operably linked to the sequence encoding the mature NCR protein. When the N-terminal 85 amino acids of the alpha mating factor precursor protein are fused to the N-terminus of a heterologous protein such as NCR, the propeptide sequence is typically dissociated from the heterologous protein via cleavage by endogenous yeast proteases at the KEX2 recognition site. The NCR protein can thus be expressed without the glu-ala repeats.

To obtain transformed yeast that express NCR peptides, the yeast NCR expression cassettes (e.g., yeast promoter, yeast signal peptide encoding sequence, mature NCR protein sequence, and polyadenylation sequence) are typically combined with other sequences that provide for selection of transformed yeast. Examples of useful selectable marker genes include genes encoding a ADE protein, a HIS5 protein, a HIS4 protein, a LEU2 protein, a URA3 protein, ARG4 protein, a TRP1 protein, a LYS2 protein, a protein conferring resistance to a bleomycin or phleomycin antibiotic, a protein conferring resistance to chloramphenicol, a protein conferring resistance to G418 or geneticin, a protein conferring resistance to hygromycin, a protein conferring resistance to methotrexate, an a ARO4-OFP protein, and a FZF1-4 protein. Similar electable marker cassettes that confer resistance to antibiotics or rescue auxotrophic traits can be used in bacterial or fungal systems.

DNA molecules comprising the yeast NCR expression cassettes and selectable marker genes are introduced into yeast cells by techniques such as transfection into yeast spheroplasts or electroporation. In certain embodiments, the DNA molecules comprising the yeast NCR expression cassettes and selectable marker genes are introduced as linear DNA fragments that are integrated into the genome of the transformed yeast host cell. Integration can occur either at random sites in the yeast host cell genome or at specific sites in the yeast host cell genome. Integration at specific sites in the yeast host cell genome is typically accomplished by homologous recombination between sequences contained in the expression vector and sequences in the yeast host cell genome. Homologous recombination is typically accomplished by linearizing the expression vector within the homologous sequence (for example, within the AOX1 promoter sequence of a Pichia expression vector when integrating the expression vector into the endogenous AOX1 gene in the Pichia host cell). In other embodiments, the yeast expression cassettes can also comprise additional sequences such as autonomous replication sequences (ARS) that provide for the replication of DNA containing the expression cassette as an extrachromosomal (non-integrated) element. Such extra-chromosomal elements are typically maintained in yeast cells by continuous selection for the presence of the linked selectable marker gene. Yeast artificial chromosomes (YACs) containing sequences that provide for replication and mitotic transmission are another type of vector that can be used to maintain the DNA construct in a yeast host.

Yeast, bacterial, or fungal cells transformed with the yeast or fungal NCR expression cassettes can be used to produce mixtures of NCR_PFV which can be used to obtain preparation enriched for a desired NCR_PFV. The desired NCR_PFV can be used as antimicrobial agents, to produce antimicrobial compositions that can be applied to plants or foodstuffs, or to produce antimicrobial compositions that can be administered to human or non-human subjects. Methods of producing mixtures of NCR_PFV can typically first comprise the step of culturing yeast, bacterial, or fungal cells transformed with NCR expression cassettes under conditions wherein the yeast cells express a mature NCR molecule. In general, the conditions where the yeast bacterial, or fungal cells express the mature NCR molecules are conditions that allow for or specifically induce expression of the yeast promoter that is operably linked to the NCR coding sequence in the yeast expression cassette. When the yeast is Pichia and the signal-peptide/MD gene is under the control of an AOX1 or AOX2 promoter, addition of methanol to the growth medium will provide for expression of mature NCR protein. Similarly, when the yeast is Hansuela and the signal-peptide/MD gene is under the control of a MOX, DHAS, or FMDH promoter, addition of methanol to the growth medium will provide for expression of mature NCR protein. Alternatively, when the yeast is Kluveromyces and the signal-peptide/De/5 gene is under the control of a Lactase promoter, addition of galactose to the growth medium will provide for expression of mature NCR protein.

Once the transformed yeast, bacterial, or fungal culture has been incubated under culture conditions that provide for expression of mature NCR peptide for a sufficient period of time, the desired NCR molecule PFV can be separated from any undesired NCR_PFV isolated from the culture as a mixture. A sufficient period of time can be determined by periodically harvesting portions or aliquots of the culture and assaying for the presence of desired and/or undesired NCR_PFV. Analytical assays such as HPLC be used to monitor desired NCR_PFV and undesired NCR_PFV production. In certain embodiments, culture incubation periods can be optimized for production of the desired NCR_PFV such that the culture is enriched for the desired NCR_PFV.

Methods according to these embodiments comprise (a) separating a fraction comprising a 6-Cys or 4-Cys NCR peptide folding variant 1 (NCR_PFV1) from one or more fractions comprising 6-Cys or 4-Cys NCR peptide folding variant 2 (NCR_PFV2), or NCR_PFV2 peptide fragments thereof, from a mixture comprising NCR_PFV1 and NCR_PFV2, or NCR_PFV2 peptide fragments thereof; and (b) combining said fraction comprising NCR_PFV1, or a preparation further purified from said NCR_PFV1 fraction, with an agriculturally, pharmaceutically, or veterinary practicable carrier, diluent, or excipient, thereby making the composition.

Mixtures comprising NCR_PFV1 and NCR_PFV2, or NCR_PFV2 peptide fragments thereof, can be prepared by (a) culturing in fermentation broth microorganisms expressing a recombinant polynucleotide comprising a transcriptional promoter operably linked to a polynucleotide encoding a signal peptide that is in frame with and upstream of a polynucleotide encoding NCR_PFV1, or a variant thereof, (b) separating (e.g., by size-exclusion chromatography) the microorganisms from the fermentation broth, which comprises the mixture, (c) disrupting the microorganisms to obtain a cell lysate, and (d) separating insoluble debris in the cell lysate from an aqueous fraction comprising said mixture.

Isolation of the desired NCR_PFV from the culture can be partial or complete. For NCR expression vectors where a yeast, bacterial, or fungal signal peptide is operably linked to the sequence encoding the mature NCR protein, a mixture comprising the desired NCR_PFV and any raNCR_PFV or other undesired NCR_PFV (e.g., increased or decreased protease resistance) can be recovered from the yeast cell culture medium, bacterial periplasm, or fungal cell culture medium. In certain embodiments, yeast or fungal cell culture medium that contains the mature NCR protein in the form of both a desired NCR_PFV and an undesired NCR_PFV can be separated from the yeast or fungal cells by centrifugation or filtration, thus providing a composition comprising a mixture of the desired and undesired NCR_PFV. In certain embodiments, preparations of bacterial periplasmic proteins that contains the mature NCR protein in the form of both a desired NCR_PFV and an undesired NCR_PFV can be obtained by methods including physical force, pressure, osmotic shock, or chemical (e.g., EDTA+heat or detergent) treatment, thus providing a composition comprising a mixture of the desired and undesired NCR_PFV.

Such methods for isolation of bacterial periplasmic proteins can be adapted from methods disclosed for isolation of other periplasmic proteins (e.g., as in Schimek et al., (2020) Biotechnology progress, 36(5), e2999. doi.org/10.1002/btpr.2999). Yeast or fungal cell culture medium or preparations of bacterial periplasmic proteins that contain the mature NCR protein can be further processed by any combination of dialysis and/or concentration techniques (e.g., precipitation, lyophilization, filtration) to produce a composition containing one or more NCR_PFV (e.g. a mixture of the desired and undesired NCR_PFV). In certain embodiments, filtration size-exclusion membranes can be used to enrich for a fraction containing a mixture of desired and undesired NCR_PFV.

Combinations of various isolation techniques can also be employed to produce a mixture of NCR_PFV. For example, the cell culture medium can be separated from the cells by centrifugation and dialyzed or adjusted. In certain embodiments, a buffer for dialysis or adjustment is a 25 mM sodium acetate buffer at about pH4.5-pH6.0. This dialysate is then subjected to ion-exchange chromatography. For example, a cation-exchange resin such as CM-Sephadex C-25 equilibrated with a 25 mM sodium acetate buffer at about pH6.0 can be used. NCR protein bound to the cation exchange resin is washed and then eluted. For example, the aforementioned column is washed with 25 mM sodium acetate buffer at about pH6.0 and subsequently eluted in 1M NaCl, 50 mM Tris, pH7.6. Fractions containing the NCR protein are identified by an assay or by UV absorbance and then concentrated by a size-cutoff filtration membrane. The concentrated NCR protein is then dialyzed to obtain an essentially or substantially pure NCR protein in a buffer. Buffers include buffers such as 10 mM Tris, pH 7.6.

Production of compositions comprising or enriched for the desired NCR_PFV can be obtained by methods which provide for separation of the desired NCR_PFV from the undesired NCR_PFV(s). In certain embodiments, such methods can comprise standard protein separation techniques which enrich for the desired NCR_PFV based on different chemical properties besides mass (e.g., the hydrodynamic radius of different NCR_PFV and/or the charge characteristics of the different NCR_PFV). In certain embodiments, chromatographic techniques such as sedimentation, size exclusion chromatography (SEC), ion-exchange chromatography, and/or affinity chromatography can be used to produce a composition enriched for the desired NCR_PFV. Methods for separating proteins having different conformations by SEC which have been disclosed can be adapted for use in separating a desired from one or more undesired NCR_PFV(s) (La Verde et al., Bio Protoc. 2017 Apr. 20; 7(8):e2230. doi: 10.21769/BioProtoc.2230).

In certain embodiments, the methods can provide for a composition comprising a desired NCR_PFV where the composition comprises less than 5%, 2%, 1%, 0.5%, 0.25%, or 0.1% of one or more undesirable NCR_PFV(s) by weight. In certain embodiments, the methods can provide for a composition comprising a desired NCR_PFV where less than 5%, 2%, 1%, or 0.5% of the combined total of the desired NCR_PFV and one or more undesired NCR_PFV(s) by weight in the composition is/are the undesired NCR_PFV(s). In certain embodiments, the methods can provide for a composition comprising a desired NCR_PFV where at least about 80%, 90%, 95%, 98%, 99%, or 99.5% of the total NCR peptide by weight or molarity in the composition is the desired NCR_PFV.

In certain embodiments, the methods can provide a composition where less than 20%, 10%, 5%, 2%, 1%, or 0.5% of the total NCR peptide in the composition by weight is undesired NCR_PFV or wherein at least about 80%, 90%, 95%, 98%, 99%, or 99.5% of the total NCR peptide in the composition by weight is the desired NCR_PFV. Total NCR peptide by weight in the composition can be determined by quantifying the combined total amount of desired and undesired NCR_PFV in the composition. Quantification of total NCR peptides and/or an isolated NCR_PFV can be accomplished by a variety of methods including immunoassays (e.g., ELISAs, RIA where a purified NCR peptide is used to establish a reference curve) or mass spectroscopy (Zhang et al. Methods Mol Biol. 2010; 673:211-222. doi:10.1007/978-1-60761-842-3_13).

In certain embodiments, compositions enriched for desired NCR_PFV can be obtained by methods which exploit the relative sensitivity of a desired NCR_PFV to a protease (e.g., a serine protease such as trypsin). In embodiments where the desired NCR_PFV exhibits increased resistance to the protease (e.g., at a given protease concentration), mixtures comprising the desired and undesired NCR_PFV can be treated with the protease at a concentration which will cleave the undesired NCR_PFV yet leave the desired NCR_PFV intact. The intact NCR_PFV can then be separated from the resultant fragments of the NCR peptide produced by cleavage of the undesired NCR_PFV by methods including size-exclusion membrane filtration, SEC, and/or IEC. Suitable proteases for such methods can include a trypsin family serine protease. In certain embodiments, the serine protease will cleave an undesired NCR_PFV at the carboxy terminus of an arginine or lysine residue in that undesired NCR_PFV under non-denaturing conditions. In certain embodiments, the trypsin family serine protease is a recombinant bovine, porcine, human, or microbial trypsin, optionally wherein the microbial trypsin is a Streptomycete trypsin or variant thereof. In certain embodiments, about 6.25, 10, or 12.5 to about 15, 20, or 25 BAEE units/mL of trypsin (e.g., Sigma-Aldrich Catalog No. T1426, St. Louis, MO) can be incubated with the NCR PFV for about 1, 2, or 3 to about 4, 5, or 6 hours at 37° C. in 60 mM monobasic sodium phosphate buffer at pH 7.5 to cleave the undesired NCR_PFV yet leave the desired NCR_PFV intact. One BAEE (Na-Benzoyl-L-arginine ethyl ester hydrochloride) trypsin substrate unit will produce a delta A253 (change in absorbance at 253 nm) of 0.001 per minute with BAEE as substrate at pH 7.6 at 25 deg C. in a reaction volume=3.2 ml (1 cm light path).

In some aspects of these methods a mixture comprising NCR_PFV1 and NCR_PFV2, or NCR_PFV2 peptide fragments is treated with a protease under non-denaturing conditions that are sufficient to produce NCR_PFV2 peptide fragments but insufficient to produce NCR_PFV1 peptide fragments. Suitable proteases include trypsin family serine proteases that cleave NCR peptides at the carboxy terminus of an Arg (R) or Lys (K). Trypsin family serine proteases include recombinant bovine, porcine, human, or microbial trypsin, and, optionally, wherein the microbial trypsin is a Streptomycete trypsin or variant thereof.

A 6-Cys or 4-Cys NCR_PFV1 according to the present embodiments can exhibit an increase in phospholipid binding in comparison to a corresponding a 6-Cys or 4-Cys NCR_PFV2. A desired 6-Cys or 4-Cys NCR_PFV1 provided herein can exhibit lower IC50 values against one or more microbial pathogens, improved binding to phospholipids, or any combination thereof in comparison to either: (i) mixtures comprising the desired 6-Cys or 4-Cys NCR_PFV1 and one or more corresponding undesired 6-Cys or 4-Cys NCR_PFV2 or (ii) the 6-Cys or 4-Cys NCR_PFV2 alone. The desired 6-Cys or 4-Cys NCR_PFV1 can be identified and/or selected for lower IC50 values against one or more microbial pathogens by selecting for 6-Cys or 4-Cys NCR_PFV1 that provide for improved phospholipid binding in comparison to in comparison to either: (i) mixtures comprising the desired 6-Cys or 4-Cys NCR_PFV1 and one or more corresponding undesired 6-Cys or 4-Cys NCR_PFV2 or (ii) the 6-Cys or 4-Cys NCR_PFV2 alone.

Suitable assays for determining improved phospholipid include protein-lipid overlay assays (e.g., Dowler et al., 2002, Sci STKE. 2002 Apr. 23; 2002(129):16), surface plasmon resonance assays (e.g., Baron and Pauron, 2014, Bio-protocol 4(18): e1237), biotin capture lipid affinity assays (e.g., Davidson et al., 2006, Lipid Research, 47, 440-449), titration calorimetry assays (e.g., Miller and Cistola, 1993 Molecular and Cellular Biochemistry, 123(1): 29-37), and the like.

Typically, the permeability of a microbial plasma membrane treated with a 6-Cys or 4-Cys NCR_PFV1 as described herein can increase in comparison to permeability of a microbial plasma membrane treated with a corresponding a 6-Cys or 4-Cys NCR_PFV2. Membrane permeability can be measured by a variety of techniques that include dye uptake. Convenient dye uptake assays that can be used to assess changes in in membrane permeability include assays for uptake of Hoechst 33342 (H0342), rhodamine 123, SYTOX™ Green, and the like. These dyes enter into microbial cells only if their plasma membrane has been permeabilized by an NCR peptide, defensin, or other membrane-permeabilizing agent. Without seeking to be limited by theory, in certain embodiments it is believed that the NCR protein comprising an NCR peptide and another antifungal peptide joined by a spacer peptide can provide improved microbial inhibition by increasing the permeability of treated microbial membranes in comparison to microbial membranes treated with an NCR peptide.

The use of microbial disease severity assays and colony formation assays in conjunction with expression assays to identify transgenic MsDefl-expressing potato plants that are resistant to Verticillium dahliae has been described (U.S. Pat. No. 6,916,970 and Gao et al., 2000, Nature Biotechnology 18, 1307-1310). It is similarly contemplated that a variety of 6-Cys or 4-Cys NCR_PFV1 treated plants that combat or control microbial pathogens can be identified by scoring treated plants for resistance to microbial pathogens that infect those plants. Examples of NCR_PFV1-conferred microbial resistance that can be assayed by observing reductions in disease symptoms or reductions in microbial growth include resistance of treated corn to Fusarium verticillioides, Fusarium moniliforme, Colletotrichum graminicola, Stenocarpella maydis, and/or Cercospora zeae-maydis; resistance of treated wheat to head blight (Fusarium graminearum), powdery mildew (Erysiphe graminis f. sp. tritici), stripe rust, stem rust or leaf rust (Puccinia tritici); resistance of treated cotton to Fusarium oxysporum and Verticillium dahlia; resistance of treated rice to Magnaporthe oryzae and Rhizoctonia solani, and resistance of treated soybean to Asian Soybean rust (Phakopsora pachyrhizi), Phytophthora Root Rot (Phytophthora sp.), White Mold (Sclerotinia sp.), Sudden Death Syndrome (Fusarium virguliforme) and/or Brown Stem Rot (Phialophora gregata).

Inhibitory amounts of a desirable 6-Cys or 4-Cys NCR_PFV1 against a pathogenic microbe in a given plant can also be identified by measuring decreases in the adverse effects cause by microbial growth in such plants. Such decreases can be ascertained by comparing the extent of the adverse effect in an NCR_PFV1 treated plant relative to an otherwise identical, control plant treated with a control composition lacking the NCR_PFV1 or comprising the corresponding NCR_PFV2. Adverse effects of microbial growth in a plant that can be measured include any type of plant tissue damage or necrosis, any type of plant yield reduction, any reduction in the value of the crop plant product, and/or production of undesirable microbial metabolites or microbial growth by-products including, but not limited to, mycotoxins. Mycotoxins comprise a number of toxic molecules produced by microbial species, including but not limited to polyketides (including aflatoxins, demethylsterigmatocystin, O-methylsterigmatocystin, etc.), fumonisins, alperisins (e.g., Als A2, Bls B2), sphingofungins (A, B, C and D), trichothecenes, fumifungins, and the like. Methods of quantitating mycotoxin levels are widely documented. Moreover, commercial kits for measurement of the mycotoxins such as aflatoxin, fumonisin, deoxynivalenol, and zearalenone are also available (VICAM, Watertown, MA, USA).

Infection of certain plants with certain plant pathogen microbes can result in distinctive effects on plant growth that are readily observed. Consequently, one can distinguish plants treated with desirable 6-Cys or 4-Cys NCR_PFV1 from a plant treated with a control lacking an NCR peptide or comprising the corresponding raNCR_PFV by simply challenging such plants with pathogenic plant microbes and observing reduction of the symptoms normally associated with such infections. Such observations are facilitated by co-infecting otherwise identical, control plants treated with a control lacking an NCR peptide or comprising the corresponding undesirable 6-Cys or 4-Cys NCR_PFV2. Identification of treated plants that control or combat microbial infection can be based on observation of decreased disease symptoms, measurement of the decreased microbial growth in the infected plant (e.g., by determining the numbers of colony forming units per gram of infected tissue) and/or by measurement of the amount of mycotoxin present in infected plant tissue.

Plants of interest include both food crop plants and biofuels or energy crop plants. The plants, parts thereof, and harvested parts thereof (e.g., grain, fruits, and vegetables) to which the methods and compositions disclosed herein can be applied can refer to plants, parts thereof, and harvested parts thereof that are either directly edible, or which produce edible products (e.g., customarily used to feed humans either directly, or indirectly through animals). Plants, parts thereof, and harvested parts thereof used herein include cereal crop (e.g., wheat, rice, maize (corn), barley, oats, sorghum, rye, and millet), legumes (e.g., peanuts, chickpeas, lentils, kidney beans, soybeans, lima beans); root or tuber crop (e.g., potatoes, sweet potatoes, and cassavas); oil crops (e.g., canola (rapeseed), wheat, peanuts, palm, coconuts, safflower, cottonseed, sunflower, flax, and olive); sugar crops (e.g., sugar cane and sugar beets); fruit crops (e.g., bananas, oranges, apples, pears, breadfruit, pineapples, strawberries, grapes, and cherries); vegetable crops and tubers (e.g., tomatoes, lettuce, carrots, melons, asparagus, Brassica sp.); nut crops (e.g., cashews, peanuts, walnuts, pistachio nuts, almonds); forage and turf grasses; forage legumes (e.g., alfalfa, clover); drug crops (e.g., Cannabis sp., coffee, cocoa, kola nut, poppy); spice and flavoring crops (e.g., vanilla, sage, thyme, anise, saffron, menthol, peppermint, spearmint, coriander), fiber crop (e.g., cotton or hemp), and biofuel crops (e.g., castor bean, Camelina, switchgrass, Miscanthus, and Jatropha) plants, parts thereof, and harvested parts thereof.

The analysis of different NCR_PFV to assign particular disulfide bonds can be performed by mass spectrometry-based methods (Weinfurtner, in Oxidative Folding of Proteins: Basic Principles, Cellular Regulation and Engineering, 2018, pp. 81-98 DOI: 10.1039/9781788013253-00081; Tang and Speicher, Current protocols in protein science vol. 96, 1 (2019): e86. doi: 10.1002/cpps.86).

Also provided are antimicrobial compositions for agricultural, pharmaceutical, or veterinary use comprising either an antimicrobial plant, or antimicrobial human or veterinary, pathogenic microbe inhibitory amount (“antimicrobial effective amount”) of one or more the present isolated, purified antimicrobial NCR_PFV peptides, or biologically functional equivalents thereof. Such compositions can comprise one, or any combination of, NCR peptides disclosed herein, and an agriculturally, pharmaceutically, or veterinary-practicably acceptable carrier, diluent, or excipient. As indicated below, other components relevant in agricultural and therapeutic contexts can be included in such compositions as well. The antimicrobial compositions can be used for inhibiting the growth of, or killing, NCR protein- or peptide-susceptible pathogenic microbes associated with plant, human or animal microbial infections. Such antimicrobial compositions can be formulated for topical administration, and applied topically to either plants, the plant environment (including soil), or humans or animals.

Agricultural compositions comprising any of the present NCR molecules alone, or in any combination, can be formulated as described in, for example, Winnacker-Kuchler (1986) Chemical Technology, Fourth Edition, Volume 7, Hanser Verlag, Munich; van Falkenberg (1972-1973) Pesticide Formulations, Second Edition, Marcel Dekker, N.Y.; and K. Martens (1979) Spray Drying Handbook, Third Edition, G. Goodwin, Ltd., London. Formulation aids, such as carriers, inert materials, surfactants, solvents, and other additives are also well known in the art, and are described, for example, in Watkins, Handbook of Insecticide Dust Diluents and Carriers, Second Edition, Darland Books, Caldwell, N.J., and Winnacker-Kuchler (1986) Chemical Technology, Fourth Edition, Volume 7, Hanser Verlag, Munich. Using these formulations, it is also possible to prepare compositions where a desired NCR_PFV is combined with other pesticidally active substances, fertilizers, and/or growth regulators, etc., in a composition (e.g., a finished formulation or tank mixes.

Whether alone or in combination with other active agents, the present antimicrobial NCR_PFV peptides can be applied at a concentration in the range of from about 0.1 g/ml to about 100 mg/ml, or from about 5 μg/ml to about 5 mg/ml, at a pH in the range of from about 3.0 to about 9.0. Such compositions can be buffered using, for example, phosphate buffers between about 1 mM and 1 M, about 10 mM to about 100 mM, or about 15 mM to about 50 mM. In the case of low buffer concentrations, a salt can be added to increase the ionic strength. In certain embodiments, NaCl in the range of from about 1 mM to about 1 M, or about 10 mM to about 100 mM, can be added.

Numerous conventional microbial antibiotics and chemical fungicides with which the present NCR_PFV can be combined are described in Worthington and Walker (1983) The Pesticide Manual, Seventh Edition, British Crop Protection Council. These include, for example, polyoxines, nikkomycines, carboxy amides, aromatic carbohydrates, carboxines, morpholines, inhibitors of sterol biosynthesis, and organophosphorous compounds. In addition, azole, triazole, and/or echinocandin fungicides can also be used. Other active ingredients which can be formulated in combination with the present antimicrobial peptides and proteins include, for example, insecticides, attractants, sterilizing agents, acaricides, nematicides, and herbicides. U.S. Pat. No. 5,421,839, which is incorporated herein by reference in its entirety, contains a comprehensive summary of the many active agents with which substances such as the present antimicrobial NCR peptides and proteins can be formulated.

The compositions provided herein can be applied in an antimicrobial effective amount, which will vary depending on such factors as, for example, the specific fungal pathogen to be controlled, the specific plant (and plant part or soil) to be treated, and the method of applying the compositions that comprise a desired NCR_PFV.

Desired NCR_PFV and biologically functional equivalents and compositions comprising the same provided herein (e.g., in the following numbered embodiments), can be used to inhibit the growth of a wide variety of susceptible microbes in plants. In certain embodiments, growth of microbes in the following genera or species can be inhibited: Alternaria (e.g., Alternaria brassicicola; Alternaria solani); Ascochyta (e.g., Ascochyta pisi); Aspergillus (e.g., Aspergillus flavus; Aspergillus fumigatus); Botrytis (e.g., Botrytis cinerea); Cercospora (e.g., Cercospora kikuchii; Cercospora zeae-maydis); Colletotrichum (e.g., Colletotrichum lindemuthianum); Diplodia (e.g., Diplodia maydis); Erysiphe (e.g., Erysiphe graminis fsp. graminis; Erysiphe graminis f.sp. hordei); Fusarium (e.g., Fusarium nivale; Fusarium oxysporum; Fusarium graminearum; Fusarium culmorum; Fusarium solani; Fusarium moniliforme; Fusarium roseum); Gaeumannomyces (e.g., Gaeumannomyces graminis fsp. tritici); Helminthosporium (e.g., Helminthosporium turcicum; Helminthosporium carbonum; Helminthosporium maydis); Macrophomina (e.g., Macrophomina phaseolina; Magnaporthe grisea); Nectria (e.g., Nectria heamatococca); Peronospora (e.g., Peronospora manshurica; Peronospora tabacina); Phakopsora (e.g., Phakopsora pachyrhizi); Phoma (e.g., Phoma betae); Phymatotrichum (e.g., Phymatotrichum omnivorum); Phytophthora (e.g., Phytophthora cinnamomi; Phytophthora cactorum; Phytophthora phaseoli; Phytophthora parasitica; Phytophthora citrophthora; Phytophthora sojae; Phytophthora infestans); Plasmopara (e.g., Plasmopara viticola); Podosphaera (e.g., Podosphaera leucotricha); Puccinia (e.g., Puccinia sorghi; Puccinia striiformis; Puccinia graminis f.sp. tritici; Puccinia asparagi; Puccinia recondita; Puccinia arachidis); Pythium (e.g., Pythium aphanidermatum; Pythium ultimum); Pyrenophora (e.g., Pyrenophora tritici-repentens); Pyricularia (e.g., Pyricularia oryzae); Rhizoctonia (e.g., Rhizoctonia solani; Rhizoctonia cerealis); Sclerotium (e.g., Sclerotium rolfsii); Sclerotinia (e.g., Sclerotinia sclerotiorum); Septoria (e.g., Septoria lycopersici; Septoria glycines; Septoria nodorum; Septoria tritici); Thielaviopsis (e.g., Thielaviopsis basicola); Uncinula (e.g., Uncinula necator); Venturia (e.g., Venturia inaequalis); and Verticillium (e.g., Verticillium dahliae; Verticillium albo-atrum).

Pharmaceutical or veterinary compositions that comprise an antimicrobial effective amount of a desired NCR_PFV and a pharmaceutically acceptable or veterinary practicable carrier are also provided. Such pharmaceutical or veterinary compositions can be used for inhibiting the growth of, or killing, susceptible pathogenic microbes that infect humans or animals, i.e., treating such fungal infections by administering to a patient or other subject in need thereof. In certain embodiments, compositions comprising NCR_PFV, can be formulated by methods adapted from those described in Remington: The Science and Practice of Pharmacy (2005), 21st Edition, University of the Sciences in Philadelphia, Lippincott Williams & Wilkins. In certain embodiments, the compositions can contain NCR_PFV at concentrations in the range of from about 0.1 μg per ml to about 100 mg per ml, or about 5 μg per ml to about 5 mg per ml, at a pH in the range of from about 3.0 to about 9.0. Such compositions can be buffered using, for example, phosphate buffers at a concentration of about 1 mM to about 1 M, about 10 mM to about 100 mM, or about 15 mM to 50 mM. In the case of low buffer concentrations, a salt can be added to increase the ionic strength. In certain embodiments, NaCl in the range of about 1 mM to about 1 M, or about 10 mM to about 100 mM, can be added.

The NCR_PFV can be formulated alone or can formulated in combination with other conventional antimicrobial therapeutic compounds such as, by way of non-limiting example, polyene antimicrobials; imidazole, triazole, and thiazole antimicrobials; allylamines; and echinocandins that are routinely used in human and veterinary medicine.

Administration of the compositions that comprise NCR_PFV to a human or animal subject in need thereof can be accomplished via a variety of routes that include topical application, enteral, parenteral, and/or intravenous introduction.

In further embodiments, the present disclosure provides methods for preventing or reducing crop damage or post-harvest loss by a plant pathogenic microbe, the method comprising contacting a plant, a plant seed, pre- or post-harvest grain, pre- or post-harvest fruit, or pre- or post-harvest vegetable with an effective amount of a composition comprising a cationic antimicrobial 6-Cys or 4-Cys nodule-specific cysteine-rich (NCR) peptide folding variant 1 (NCR_PFV1) under conditions that are suitable for preventing or reducing crop damage or post-harvest loss.

In other embodiments, the present disclosure provides medical devices in operable combination with a composition comprising a cationic antimicrobial 6-Cys or 4-Cys nodule-specific cysteine-rich (NCR) peptide folding variant 1 (NCR_PFV1), wherein the medical device comprises at least one surface that is topically coated or impregnated with the composition.

In still further embodiments, provided herein are methods for treating, preventing, or inhibiting a microbial infection in a subject (including a plant, human, or non-human animal) in need thereof, which methods comprise administering (ex vivo or in vivo) to the subject a therapeutically effective amount of a composition comprising a cationic antimicrobial 6-Cys or 4-Cys nodule-specific cysteine-rich (NCR) peptide folding variant 1 (NCR_PFV1) under conditions and for a duration suitable for treating, preventing, or inhibiting the microbial infection.

In related embodiments, provided herein are compositions comprising a cationic antimicrobial 6-Cys or 4-Cys nodule-specific cysteine-rich (NCR) peptide folding variant 1 (NCR_PFV1) for use in a method for treating, preventing, or inhibiting microbial infection in a subject in need thereof.

In other related embodiments, provided herein are plant parts that are at least partly coated with a composition comprising a cationic antimicrobial 6-Cys or 4-Cys nodule-specific cysteine-rich (NCR) peptide folding variant 1 (NCR_PFV1).

### EMBODIMENTS

The following numbered embodiments form part of the present disclosure.

1a. A composition comprising a nodule-specific cysteine rich NCR peptide folding variant 1 (NCR_PFV1), wherein the NCR peptide is a cationic 6-Cys NCR peptide comprising in contiguous amino acid sequence from its N-terminal end (1) a first cysteine pair motif including C₁ and C₂, (2) a first intervening sequence (IS1), (3) a second cysteine pair motif including C₃ and C₄, (4) a second intervening sequence (IS2), and (5) a third cysteine pair motif including C₅ and C₆, wherein the NCR peptide has less than 60%, 70%, 80%, 85%, 90%, 92%, 95%, 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4), wherein the cysteine residues C₁, C₂, C₃, C₄, C₅, and C₆ of NCR_PFV1 form a first set of disulfide bonds and NCR_PFV1 thereby adopts a first conformation that exhibits greater antimicrobial activity as compared to a nodule-specific cysteine rich NCR peptide folding variant 2 (NCR_PFV2) that has the same amino acid sequence as NCR_PFV1 but forms a distinct second set of disulfide bonds between cysteine residues C₁, C₂, C₃, C₄, C₅, and C₆ and adopts a distinct second conformation, and wherein the composition is substantially free of NCR_PFV2, optionally wherein at least about 80%, 90%, 95%, 98%, 99%, or 99.5% of the total NCR peptide in the composition by weight is DEF_PFV1.

1b. A composition comprising a nodule-specific cysteine rich NCR peptide folding variant 1 (NCR_PFV1), wherein the NCR peptide is a cationic 6-Cys NCR peptide comprising in contiguous amino acid sequence from its N-terminal end (1) a first cysteine pair motif including C₁ and C₂, (2) a first intervening sequence (IS1), (3) a second cysteine pair motif including C₃ and C₄, (4) a second intervening sequence (IS2), and (5) a third cysteine pair motif including C₅ and C₆, wherein the NCR peptide has less than 60%, 70%, 80%, 85%, 90%, 92%, 95%, 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4), wherein the cysteine residues C₁, C₂, C₃, C₄, C₅, and C₆ of NCR_PFV1 form a first set of disulfide bonds and NCR_PFV1 thereby adopts a first conformation that exhibits greater antimicrobial activity increased protease resistance, or increased protease sensitivity as compared to a nodule-specific cysteine rich NCR peptide folding variant 2 (NCR_PFV2) that has the same amino acid sequence as NCR_PFV1 but forms a distinct second set of disulfide bonds between cysteine residues C₁, C₂, C₃, C₄, C₅, and C₆ and adopts a distinct second conformation, and wherein the composition is substantially free of NCR_PFV2, optionally wherein at least about 80%, 90%, 95%, 98%, 99%, or 99.5% of the total NCR peptide in the composition by weight is DEF_PFV1.

2. The composition of embodiment 1a or 1b, wherein the first set of disulfide bonds comprises a C₁-C₄ disulfide bond, a C₂-C₅ disulfide bond, and a C₃-C₆ disulfide bond.

3. The composition of embodiment 2, wherein the second set of disulfide bonds comprises disulfide bonds other than a C₁-C₄ disulfide bond and a C₂-C₅ disulfide bond.

4. The composition of embodiment 3, wherein the second set of disulfide bonds comprises a C₁-C₂ disulfide bond, a C₃-C₆ disulfide bond, and a C₄-C₅ disulfide bond.

5. The composition of any one of embodiments 1a or 1b to 4, wherein the cationic 6-Cys NCR has an isoelectric point between 8.0 and 12.0.

6. The composition of any one of embodiments 1a or 1b to 5, wherein the NCR_PFV1 exhibits greater protease resistance than the NCR_PFV2.

7. The composition of any one of embodiments 1a or 1b to 6, wherein the first cysteine pair motif is a C₁-5AA-C₂ motif that comprises in contiguous sequence from its N-terminal end five amino acids (A₁, A₂, A₃, A₄, and A₅) flanked by an N-terminal C₁ and a C-terminal C₂.

8. The composition of any one of embodiments 1a or 1b to 6, wherein the A₅ is an anionic amino acid selected from the group consisting of Asp (D) and Glu (E) or a cationic amino acid selected from the group consisting of His (H), Arg (R), and Lys (K) or an aliphatic amino acid selected from the group consisting of Gly (G), Ala (A), Val (V), Leu (L), Ile (I), and Pro (P).

9. The composition of any one of embodiments 1a or 1b to 6, wherein the A₄ is a cationic amino acid selected from the group consisting of His (H), Arg (R), and Lys (K) or an aliphatic amino acid selected from the group consisting of Gly (G), Ala (A), Val (V), Leu (L), Ile (I), and Pro (P) or an amide amino acid selected from the group consisting of Asn (N) and Gln (Q) or a polar amino acid selected from the group consisting of Ser (S) and Thr (T).

10. The composition of any one of embodiments 1a or 1b to 6, wherein the A₃ is a cationic amino acid selected from the group consisting of His (H), Arg (R), and Lys (K) or an anionic amino acid selected from the group consisting of Asp (D) and Glu (E) or a polar amino acid selected from the group consisting of Ser (S) and Thr (T).

11. The composition of any one of embodiments 1a or 1b to 6, wherein the A₅ is an anionic amino acid selected from the group consisting of Asp (D) and Glu (E), wherein the A₄ is a cationic amino acid selected from the group consisting of His (H), Arg (R), and Lys (K) and wherein the A₃ is an anionic amino acid selected from the group consisting of Asp (D) and Glu (E).

12. The composition of any one of embodiments 1a or 1b to 6, wherein the second cysteine pair motif is a C₃-5-10AA-C₄ motif that comprises in contiguous sequence from its N-terminal end from five to ten amino acids (A₆, A₇, A₈, A₉, A₁₀ up through A₁₁, A₁₂, A₁₃, A₁₄, A₁₅) flanked by an N-terminal C₃ and a C-terminal C₄.

13. The composition of any one of embodiments 1a or 1b to 6, wherein the second cysteine pair motif is a C₃-5-10AA-C₄ motif that comprises in contiguous sequence from its N-terminal end seven amino acids (A₆, A₇, A₈, A₉, A₁₀, A₁₁, and A₁₂) flanked by an N-terminal C₃ and a C-terminal C₄.

14. The composition of any one of embodiments 1a or 1b to 6, wherein the A₆ is an aliphatic amino acid selected from the group consisting of Gly (G), Ala (A), Val (V), Leu (L), Ile (I), and Pro (P) or a cationic amino acid selected from the group consisting of His (H), Arg (R), and Lys (K).

15. The composition of any one of embodiments 1a or 1b to 6, wherein the A₁₂ is an anionic amino acid selected from the group consisting of Asp (D) and Glu (E) or a cationic amino acid selected from the group consisting of His (H), Arg (R), and Lys (K) or an amide amino acid selected from the group consisting of Asn (N) and Gln (Q).

16. The composition of any one of embodiments 1a or 1b to 6, wherein the A₆ and the A₁₂ are cationic amino acids selected from the group consisting of His (H), Arg (R), and Lys (K).

17. The composition of any one of embodiments 1a or 1b to 6, wherein the third cysteine pair motif is a C₅-1AA-C₆ motif that comprises one amino acid (A₁₆) flanked by an N-terminal C₅ and a C-terminal C₆.

18. The composition of any one of embodiments 1a or 1b to 6, wherein the A₁₆ is a cationic amino acid selected from the group consisting of His (H), Arg (R), and Lys (K) or an aliphatic amino acid selected from the group consisting of Gly (G), Ala (A), Val (V), Leu (L), Ile (I), and Pro (P) or an anionic amino acid selected from the group consisting of Asp (D) and Glu (E).

19. The composition of any one of embodiments 1a or 1b to 6, wherein the A₁₆ is a cationic amino acid selected from the group consisting of His (H), Arg (R), and Lys (K).

20. The composition of any one of embodiments 1a or 1b to 6, wherein the IS1 comprises from one to eight amino acids or from four to five amino acids.

21. The composition of any one of embodiments 1a or 1b to 6, wherein the IS2 comprises from four to six amino acids.

22. The composition of any one of embodiments 1a or 1b to 6, wherein the IS1 and the IS2 each consists of four amino acids.

23. The composition of any one of embodiments 1 to 22, wherein the NCR_PFV1 comprises an amino acid sequence having at least 70%, 75%, 80%, 85%, 90%, 95%, 97%, or 99% sequence identity to any one of SEQ ID NO: 8 to 371.

24. The composition of any one of embodiments 1 to 23, wherein the composition further comprises an agriculturally, pharmaceutically, or veterinary practicable carrier, diluent, or excipient.

25a. A composition comprising a nodule-specific cysteine rich NCR peptide folding variant 1 (NCR_PFV1), wherein the NCR peptide is a cationic 4-Cys NCR comprising in contiguous amino acid sequence from its N-terminal end (1) a first cysteine pair motif including C₁ and C₂, (2) a first intervening sequence (IS1), and (3) a second cysteine pair motif including C₃ and C₄, wherein the NCR peptide has less than 60%, 70%, 80%, 85%, 90%, 92%, 95%, 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4), wherein the NCR_PFV1 cysteine residues C₁, C₂, C₃, and C₄ form a first set of disulfide bonds and NCR_PFV1 thereby adopts a first conformation that exhibits greater antimicrobial activity as compared to a nodule-specific cysteine rich NCR peptide folding variant 2 (NCR_PFV2) that has the same amino acid sequence as NCR_PFV1 but forms a distinct second set of disulfide bonds between cysteine residues C₁, C₂, C₃, C₄, C₅, and C₆ and adopts a distinct second conformation, and wherein the composition is substantially free of NCR_PFV2, optionally wherein at least about 80%, 90%, 95%, 98%, 99%, or 99.5% of the total NCR peptide in the composition by weight is NCR_PFV1.

25b. A composition comprising a nodule-specific cysteine rich NCR peptide folding variant 1 (NCR_PFV1), wherein the NCR peptide is a cationic 4-Cys NCR comprising in contiguous amino acid sequence from its N-terminal end (1) a first cysteine pair motif including C₁ and C₂, (2) a first intervening sequence (IS1), and (3) a second cysteine pair motif including C₃ and C₄, wherein the NCR peptide has less than 60%, 70%, 80%, 85%, 90%, 92%, 95%, 97%, 98%, 99%, or 100% sequence identity to CaNCR7 (SEQ ID NO: 2), CaNCR13 (SEQ ID NO: 1), CaNCR14 (SEQ ID NO: 3), or CaNCR15 (SEQ ID NO: 4), wherein the NCR_PFV1 cysteine residues C₁, C₂, C₃, and C₄ form a first set of disulfide bonds and NCR_PFV1 thereby adopts a first conformation that exhibits greater antimicrobial activity, increased protease resistance, or increased protease sensitivity as compared to a nodule-specific cysteine rich NCR peptide folding variant 2 (NCR_PFV2) that has the same amino acid sequence as NCR_PFV1 but forms a distinct second set of disulfide bonds between cysteine residues C₁, C₂, C₃, C₄, C₅, and C₆ and adopts a distinct second conformation, and wherein the composition is substantially free of NCR_PFV2, optionally wherein at least about 80%, 90%, 95%, 98%, 99%, or 99.5% of the total NCR peptide in the composition by weight is NCR_PFV1.

26. The composition of embodiment 25a or 25b, wherein the first set of disulfide bonds comprises a C₁-C₂ disulfide bond and a C₃-C₄ disulfide bond.

27. The composition of embodiment 26, wherein the second set of disulfide bonds comprises disulfide bonds other than a C₁-C₂ disulfide bond and a C₃-C₄ disulfide bond.

28. The composition of embodiment 27, wherein the second set of disulfide bonds comprises a C₁-C₃ disulfide bond and a C₂-C₄ disulfide bond.

29. The composition of embodiment 25, wherein the first set of disulfide bonds comprises a C₁-C₃ disulfide bond and a C₂-C₄ disulfide bond.

30. The composition of embodiment 29, wherein the second set of disulfide bonds comprises disulfide bonds other than a C₁-C₃ disulfide bond and a C₂-C₄ disulfide bond.

31. The composition of embodiment 25a or 25b, wherein the first set of disulfide bonds comprises a C₁-C₄ disulfide bond and a C₂-C₃ disulfide bond.

32. The composition of embodiment 31, wherein the second set of disulfide bonds comprises disulfide bonds other than a C₁-C₄ disulfide bond and a C₂-C₃ disulfide bond.

33. The composition of any one of embodiments 25a or 25b to 32, wherein the cationic 4-Cys NCR has an isoelectric point from pH 8.0 to 12.0.

34. The composition of any one of embodiments 25a or 25b to 33, wherein the NCR_PFV1 exhibits greater protease resistance as compared to the NCR_PFV2.

35. The composition of any one of embodiments 25 a or 25b to 33, wherein the first cysteine pair motif is a C₁-5AA-C₂ motif that comprises in contiguous sequence from its N-terminal end five amino acids (A₁, A₂, A₃, A₄, and A₅) flanked by an N-terminal C₁ and a C-terminal C₂.

36. The composition of any one of embodiments 25 a or 25b to 33, wherein the second cysteine pair motif is a C₃-4AA-C₄ motif that comprises in contiguous sequence from its N-terminal end four amino acids (A₆, A₇, A₈, and A₉) flanked by an N-terminal C₃ and a C-terminal C₄, and

37. The composition of any one of embodiments 25a or 25b to 33, wherein the IS1 comprises from five to thirteen amino acids or from five to eleven amino acids.

38. The composition of any one of embodiments 25a or 25b to 33, wherein the A₅ is an anionic amino acid selected from the group consisting of Asp (D) and Glu (E).

39. The composition of any one of embodiments 25a or 25b to 33, wherein the A₄ is a cationic amino acid selected from the group consisting of His (H), Arg (R), and Lys (K).

40. The composition of any one of embodiments 25a or 25b to 33, wherein the A₃ is an anionic amino acid selected from the group consisting of Asp (D) and Glu (E).

41. The composition of any one of embodiments 25a or 25b to 33, wherein the A₆ is a cationic amino acid selected from the group consisting of His (H), Arg (R), and Lys (K) or an aliphatic amino acid selected from the group consisting of Gly (G), Ala (A), Val (V), Leu (L), Ile (I), and Pro (P).

42. The composition of any one of embodiments 25a or 25b to 33, wherein the A₇ is a cationic amino acid selected from the group consisting of His (H), Arg (R), and Lys (K) or an amide amino acid selected from the group consisting of Asn (N) and Gln (Q) or an aliphatic amino acid selected from the group consisting of Gly (G), Ala (A), Val (V), Leu (L), Ile (I), and Pro (P).

43. The composition of any one of embodiments 25a or 25b to 33, wherein the A₈ is an aliphatic amino acid selected from the group consisting of Gly (G), Ala (A), Val (V), Leu (L), Ile (I), and Pro (P) or an amide amino acid selected from the group consisting of Asn (N) and Gln (Q) or a cationic amino acid selected from the group consisting of His (H), Arg (R), and Lys (K).

44. The composition of any one of embodiments 25a or 25b to 33, wherein the NCR_PFV1 comprises an amino acid sequence having at least 60%, 70%, 75%, 80%, 85%, 90%, 95%, 97%, or 99% sequence identity to any one of SEQ ID NO: 8 to 371, optionally wherein the NCR_PFV1 does not comprise the amino acid sequence of NCR169 (SEQ ID NO:6) or NCR247 (SEQ ID NO: 5).

45. The composition of any one of embodiments 25a or 25b to 33, wherein the composition further comprises an agriculturally, pharmaceutically, or veterinary practicable carrier, diluent, or excipient.

46. A method for making a composition of any one of embodiments 1a or 1b to 4 or 25a or 25b to 45, the method comprising: (a) separating a fraction comprising NCR peptide folding variant 1 (NCR_PFV1) from one or more fractions comprising NCR peptide folding variant 2 (NCR_PFV2), or NCR_PFV2 peptide fragments thereof, from a mixture comprising NCR_PFV1 and NCR_PFV2, or NCR_PFV2 peptide fragments thereof; and (b) combining the fraction comprising NCR_PFV1, or a preparation further purified from the NCR_PFV1 fraction, with an agriculturally, pharmaceutically, or veterinary practicable carrier, diluent, or excipient, thereby making the composition.

47. The method of embodiment 46, further comprising obtaining the mixture comprising NCR_PFV1 and NCR_PFV2, or NCR_PFV2 peptide fragments thereof, by (a) culturing in fermentation broth microorganisms expressing a recombinant polynucleotide comprising a transcriptional promoter operably linked to a polynucleotide encoding a signal peptide that is in frame with and upstream of a polynucleotide encoding NCR_PFV1, or a variant thereof, and (b) separating the microorganisms from the fermentation broth, which comprises the mixture, wherein the microorganisms are optionally selected from the group consisting of Candida, Kluveromyces, Hansenula, Pichia, Saccharomyces, Schizosaccharomyces, or Yarrowia, Myceliophthora, and Escherichia.

48. The method of embodiment 46, further comprising obtaining the mixture comprising NCR_PFV1 and NCR_PFV2, or NCR_PFV2 peptide fragments thereof, by (a) culturing in fermentation broth microorganisms expressing a recombinant polynucleotide comprising a transcriptional promoter operably linked to a polynucleotide encoding NCR_PFV1, or a variant thereof, and (b) separating the microorganisms, which comprises the mixture, from the fermentation broth, (c) disrupting the microorganisms to obtain a cell lysate, and (d) separating insoluble debris in the cell lysate from an aqueous fraction comprising the mixture, wherein the microorganisms are optionally selected from the group consisting of Candida, Kluveromyces, Hansenula, Pichia, Saccharomyces, Schizosaccharomyces, or Yarrowia, Myceliophthora, and Escherichia.

49. The method of embodiment 47 or 48, further comprising treating the mixture comprising NCR_PFV1 and NCR_PFV2 with a protease under non-denaturing conditions that are sufficient to produce NCR_PFV2 peptide fragments but insufficient to produce NCR_PFV1 peptide fragments.

50. The method of embodiment 49, wherein the protease is a trypsin family serine protease that cleaves NCR_PFV2 at the carboxy terminus of an NCR_PFV2 Arg (R) or Lys (K).

51. The method of embodiment 50, wherein the trypsin family serine protease is a recombinant bovine, porcine, human, or microbial trypsin, and, optionally, wherein the microbial trypsin is a Streptomycete trypsin or variant thereof.

52. The method of embodiment 49, further comprising separating the NCR_PFV1 from the NCR_PFV2 peptide fragments by size-exclusion chromatography.

53. A method for preventing or reducing crop damage or post-harvest loss by a plant pathogenic microbe, the method comprising contacting a plant, a plant seed, pre- or post-harvest grain, pre- or post-harvest fruit, or pre- or post-harvest vegetable with an effective amount of the composition of any one of embodiments 1a or 1b to 24 or 25a or 25b to 44 and under conditions suitable for preventing or reducing crop damage or post-harvest loss.

54. The method of embodiment 53, wherein the plant pathogenic microbe is selected from the group consisting of Fusarium sp., Alternaria sp., Verticillium sp., Phytophthora sp., Colletotrichum sp., Botrytis sp., Cercospora sp., Phakopsora sp., Rhizoctonia sp., Sclerotinia sp., Pythium sp., Phoma sp., Leptosphaeria sp., Gaeumannomyces sp., Puccinia sp., Septoria sp., Penicillium sp., Lasiodiplodia sp., Phomopsis sp., Mycosphaerella sp., Golovinomyces sp., Erisyphe sp., Alhugo sp., Setosphaeria sp., Cochiobolus sp., Helminthosporium sp., Diplodia sp. or Stenocarpella sp.

55. The method of embodiment 53, wherein the plant, plant seed, pre- or post-harvest grain, pre- or post-harvest fruit, or pre- or post-harvest vegetable is selected from the group consisting of a cereal crop, a legume crop, a root or tuber crop, an oil crop, a fruit crop, a vegetable crop, a nut crop, a forage or turf grass crop, a forage legume, a drug crop, a spice or flavoring crop, a fiber crop, and a biofuel crop plant, a plant seed, pre- or post-harvest grain, pre- or post-harvest fruit, or pre- or post-harvest vegetable.

56. The method of embodiment 53, wherein the plant, a plant seed, pre- or post-harvest grain, pre- or post-harvest fruit, or pre- or post-harvest vegetable is selected from the group consisting of wheat, rice, corn, barley, oat, sorghum, rye, millet, peanut, chickpea, pigeon pea, lentil, kidney bean, soybean, lima bean, potato, sweet potato, cassava, canola, peanut, palm, coconut, safflower, cotton, sunflower, flax, olive, sugarcane, sugarbeet, banana, orange, apple, pear, breadfruit, pineapple, strawberry, grape, cherry, coffee, coco, tomato, lettuce, carrot, melon, asparagus, Brassica sp., cashew, walnut, pistachio, almond, alfalfa, clover, castor, Camelina, pennycress, switchgrass, Miscanthus, and Jatropha plant, plant seed, pre- or post-harvest grain, pre- or post-harvest fruit, or pre- or post-harvest vegetable.

57. A medical device having antimicrobial properties, the medical device comprising a medical device in operable combination with a composition of any one of embodiments 1a or 1b to 24 or 25a or 25b to 44, wherein the medical device comprises at least one surface that is topically coated or impregnated with the composition.

58. The medical device of embodiment 57, wherein the medical device is selected from the group consisting of a stent, a catheter, a contact lens, a condom, a patch, and a diaphragm.

59. A method for treating, preventing, or inhibiting a microbial infection in a subject in need thereof, the method comprising administering to the subject a therapeutically effective amount of a composition of any one of embodiments 1a or 1b to 4 or 25a or 25b to 44 under conditions and for a duration suitable for treating, preventing, or inhibiting the microbial infection.

60. The method of embodiment 59, wherein the administering comprises topical, enteral, parenteral, or intravenous introduction of the composition to the subject.

61. The method of embodiment 59, wherein the subject is selected from the group consisting of human, livestock, poultry, fish, and companion animal.

62. The method of embodiment 59, wherein the microbial infection is of a mucosal membrane, eye, skin, or a nail and the composition is applied to the mucosal membrane, eye, skin, or nail.

63. The method of embodiment 59, wherein the microbial infection is by a dermatophyte, and wherein the dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

64. The method of embodiment 59, wherein the microbial infection is by a microorganism selected from the group consisting of Aspergillus, Cryptococcus, Penicillium Rhizopus, Apophysomyces, Cunninghamella, Saksenaea, Rhizomucor, Syncephalostrum, Cokeromyces, Actinomucor, Pythium, Fusarium, Histoplasmosis, and Blastomyces species.

65. The method of embodiment 64, wherein the microbial infection is by a microorganism selected from the group consisting of Candida species, wherein the Candida species is selected from the group consisting of Candida albicans (C. albicans), C. auris, C. glabrata, C. parasilosis, C. tropicalis, and C. krusei.

66. The composition of any one of embodiments 1a or 1b to 24 or 25a or 25b to 44 for use in a method for treating, preventing, or inhibiting microbial infection in a subject in need thereof.

67. The composition of embodiment 66, wherein the subject is selected from the group consisting of human, livestock, poultry, fish, and companion animals.

68. A plant part that is at least partly coated with the composition of any one of embodiments 1a or 1b to 24 or 25a or 25b to 44.

69. The plant part of embodiment 68, wherein the plant part is selected from the group consisting of a seed and a pre- or post-harvest grain.

70. The plant part of embodiment 68, wherein the plant part is selected from the group consisting of pre- or post-harvest fruit, pre- or post-harvest vegetable, and pre- or post-harvest flower.

71. The plant part of embodiment 68, wherein the plant part is selected from the group consisting of cereal crop, legume crop, root or tuber crop, oil crop, fruit crop, vegetable crop, nut crop, forage or turf grass crop, forage legume, drug crop, spice or flavoring crop, fiber crop, and biofuel crop plant part.

72. The plant part of embodiment 68, wherein the plant part is selected from the group consisting of wheat, rice, corn, barley, oat, sorghum, rye, millet, peanut, chickpea, pigeon pea, lentil, kidney bean, soybean, lima bean, potato, sweet potato, cassava, canola, peanut, palm, coconut, safflower, cotton, sunflower, flax, olive, sugarcane, sugarbeet, banana, orange, apple, pear, breadfruit, pineapple, strawberry, grape, cherry, coffee, coco, tomato, lettuce, carrot, melon, asparagus, Brassica sp., cashew, walnut, pistachio, almond, alfalfa, clover, castor, Camelina, pennycress, switchgrass, Miscanthus, and Jatropha plant part.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where a term is provided in the singular, other embodiments described by the plural of that term are also provided. As used herein, the terms “include,” “includes,” and “including” are to be construed as at least having the features to which they refer while not excluding any additional unspecified features. It will be understood that, unless indicated to the contrary, terms intended to be “open” (e.g., the term “including” should be interpreted as “including but not limited to,” the term “having” should be interpreted as “having at least,” the term “includes” should be interpreted as “includes but is not limited to,” etc.). Phrases such as “at least one,” and “one or more,” and terms such as “a” or “an” include both the singular and the plural.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub-ranges and that language such as “between,” “up to,” “at least,” “greater than,” “less than,” and the like include the number recited in the range and includes each individual member.

All references cited herein, whether supra or infra, including, but not limited to, patents, patent applications, and patent publications, whether U.S., PCT, or non-U.S. foreign, and all technical and/or scientific publications are hereby incorporated by reference in their entirety.

### EXAMPLES

While various embodiments have been disclosed herein, other embodiments will be apparent to those skilled in the art. The various embodiments disclosed herein are for purposes of illustration and are not intended to be limiting, with the true scope and spirit being indicated by the claims. The present disclosure is further described with reference to the following examples, which are provided to illustrate certain embodiments and are not intended to limit the scope of the present disclosure or the subject matter claimed.

### Example 1: Heterologous Expression and Purification of NCR_PFV1 and NCR_PFV2

A synthetic NCR gene encoding a 6-Cys or 4-Cys NCR peptide (see, Tables 1 and 2, respectively) is cloned into a linearized pPICZa-A integration vector (Invitrogen, Carlsbad, CA) in-frame with the α-mating factor secretion signal sequence containing KEX2 cleavage site without the Glu-Ala repeats and expressed in Pichiapastoris.

6-Cys or 4-Cys NCR peptides are purified using CM-Sephadex C-25 cation-exchange chromatography and C18 reverse-phase HPLC as described, with minor modifications, in Sagaram, PLoS One 6(4):e18550 (2011). After induction, cells are harvested by centrifugation at 6,000 rpm at 4° C. for 20 minutes, and the pH of the supernatant is adjusted to 6.0. A cation-exchange resin (CM-Sephadex C-25, Sigma, Cat no: C25120) is equilibrated with binding buffer (25 mM sodium acetate, pH 6.0), added to the supernatant, and incubated overnight at 4° C. at 110 rpm. After collecting and washing the resin with a binding buffer, bound proteins are eluted with elution buffer (1M NaCl, 50 mM Tris, pH 7.6) using AKTA FPLC. An FPLC fraction containing 6-Cys or 4-Cys NCR peptide is concentrated using an Amicon Ultra-15 Centrifugal filter unit. Concentrated fractions are dialyzed against 10 mM Tris, pH 7.6 and further purified by reverse phase C18-HPLC.

Peaks are observed and fractions containing NCR_PFV1 (Peak1) and NCR_PFV2 (Peak2) are collected using the method provided in the Agilent HPLC system manual (Agilent Technologies Part No. G1380-90000 (1999)). HPLC fractions containing NCR_PFV1 (Peak1) and NCR_PFV2 (Peak2) peptides are lyophilized and resuspended in nuclease-free water. The concentration of NCR_PFV1 and NCR_PFV2 are determined using a BCA assay performed according to the manufacturer's protocol (Thermo-Fisher Scientific, Inc.® “Protein Assay Technical Handbook”). The purity and size of NCR_PFV1 and NCR_PFV2 are verified by sodium dodecyl sulfate-polyacrylamide gel electrophoresis (SDS-PAGE). NCR_PFV1 and NCR_PFV2 peptides are subjected to NMR analysis or protease treatment and LC-MS to determine the disulfide bonding pairs present in each peptide.

### Example 2: Fungal Cultures and Spore Suspensions

Fungal strains of Botrytis cinerea T-4 are cultured in 20% V8 growth medium. Alternaria alternata is grown on potato dextrose agar. Fungal spores are harvested by flooding fungal growth plates with sterile water. A spore suspension is filtered through two layers of Miracloth, centrifuged at 13,600 rpm for 1 min, washed, and re-suspended in low-salt Synthetic Fungal Medium (SFM) (see, U.S. Pat. No. 6,916,970). Spore suspensions are adjusted to equivalent spore density using a hemocytometer.

Cercospora sojina and Alternaria alternata conidia are harvested from a fully grown culture plate by gently dislodging the conidia with an L-spreader in the presence of 5 ml sterile water. To remove mycelial fragments, a spore suspension is filtered through three layers of miracloth and washed twice with sterile water at 13,000 rpm for 2 minutes. Spore concentration is adjusted to 1×10⁵ spores/ml using a hemocytometer.

Sclerotinia sclerotiorum (S. sclerotiorum) 555 fungal cultures are grown at room temperature on potato dextrose agar (PDA) medium (BD Diagnostics, Sparks, MD “Difco™ & BBLTMManual, Second Edition”).

### Example 3: Determination of Minimum Inhibitory Concentration (MIC) of 6-Cys or 4-Cys NCR_PFV1 and NCR_PFV2 in Absence and Presence of Cations

Antifungal activity of 6-Cys or 4-Cys NCR_PFV1 and NCR_PFV2 is assessed at different concentrations using a 2-fold dilution series of each peptide. Antifungal activity of each peptide is determined spectrophotometrically using the 96-well plate assay (Sagaram, PLoS One 6(4):e18550 (2011) and Sagaram, PLoS One 8(12):e82485 (2013)). Around 45 μL of each peptide at different concentrations is added to each well of the microtiter plate containing 45 μL of ~10⁵ spores/ml spore suspension. Quantitative fungal growth inhibition is determined by measuring the absorbance at 595 nm using a microplate reader (Tecan Infinite® M200 Pro, Tecan Systems Inc., San Jose, CA) after 48 h. Fungal cell viability is determined by a resazurin cell viability assay (see, Li, MPMI 32:1649-1664 (2019) and Velivelli, PNAS 117(27):16043 (2020).

After incubation of a pathogen/peptide mixture for 48 h, 10 μl of 0.1% resazurin solution is added to each well. After incubation of the mixture overnight, a change in the color of the resazurin dye from blue to pink or colorless indicates the presence of live fungal cells. The MIC for each peptide is the lowest concentration of each peptide at which no change in blue color occurs. Using this protocol, the MIC value of 6-Cys or 4-Cys NCR_PFV1 and NCR_PFV2 is determined in presence of 100 mM NaCl and 2 mM CaCl₂.

MIC assays of 6-Cys or 4-Cys NCR_PFV1 and NCR_PFV2 are carried out against B. cinerea in 2× SFM to determine antifungal activity. It has been hypothesized antifungal peptides significantly lose their antifungal activity in presence of cations since electrostatic interactions between a positively charged peptide and negatively charged fungal membranes are significantly weakened in presence of cations. Antifungal activity of 6-Cys or 4-Cys NCR_PFV1 and NCR_PFV2 can, therefore, be determined in SFM supplemented with 100 mM NaCl or 2 mM CaCl₂. Loss of antifungal activity in the presence of either NaCl or CaCl₂ indicates that antifungal activity is salt-sensitive.

### Example 4: Semi-In Planta Antifungal Activity of 6-Cys or 4-Cys NCR_PFV Against S. sclerotiorum 555

For semi-in planta antifungal assays, a 1 mm plug is taken from the leading edge of freshly cultured 2-day old fungal colony from PDA media and placed on approximately 3-4 weeks of age detached soybean leaves and pods (6 weeks of age). Then, 40 μL solution of NCR_PFV at various concentrations or water is applied immediately on the plug and allowed to incubate under high humidity for 2-3 days prior to assessment. The severity of disease lesions on each leaf is assessed using the CropReporter system as described in Li et al. Mol Plant Microbe Interact. 2019 December; 32(12):1649-1664. doi: 10.1094/MPMI-08-19-0224-R. Epub 2019 Oct. 28. PMID: 31425003. High-resolution fluorescence images can be taken using CropReporter (PhenoVation, Wageningen, Netherlands).

### Example 5: Antifungal Activity of 6-Cys or 4-Cys NCR_PFV1 and PFV2 Against Botrytis cinerea Infection in Tomato Fruits

Antifungal activity assays are performed on commercially available tomato fruits. Tomato fruits are washed with 0.01% bleach and rinsed three times with sterile distilled water. Carborundum Powder (Fisher Chemical, USA) is gently rubbed on the fruit surface to create microinjuries. The fruits are inoculated with 20 μl B. cinerea conidia (at about 1×10⁵ conidia ml-1) via drop inoculation. Conidia of B. cinerea are prepared as described above. Fruits are incubated in a Ziploc weather shield box containing moist paper towels to maintain high humidity after drop inoculation. 20 μl of peptide solution (0.75 μM of 6-Cys or 4-Cys NCR_PFV1 or NCR_PFV2) is applied to the drop inoculated conidial suspension at 12 hours post-inoculation (hpi) and incubated for 5 days. The fruits are observed for fungal growth daily.

### Example 6: NCR169 Expression in Pichia pastoris Generates Only One Disulfide Variant with No Antifungal Activity

Codon optimized synthetic gene of NCR169 (SEQ ID NO: 6) cloned into pPICZalphaA vector was received from GenScript. The encoded peptide has extra alanine residue at its N-terminus (i.e., comprises the polypeptide of SEQ ID NO: 6 with an extra alanine residue at its N-terminus. The recombinant plasmid was linearized with SacI and used for transformation in Pichia pastoris X-33. A single colony was inoculated into 50 mL of YPD broth and grown overnight at 30° C. on a rotary shaker at 225 rpm. The Pichia culture was then inoculated into 500 mL of buffered minimal glycerol media and grown overnight at 30° C. on a rotary shaker at 225 rpm After 48 hrs of growth, cells were pelleted by centrifugation at 3,500 rpm for 20 minutes at room temperature (RT) and re-suspended in 1,000 mL of buffered methanol complex medium. The culture was grown for 5 days at 25° C., and 5 mL of methanol was added to the culture every 24 hours to maintain induction of gene expression. After induction, cells were pelleted by centrifugation at 3,000 rpm for 10 minutes, and the supernatant was retained. The pH of the supernatant was adjusted to 6.0 using 10 M NaOH and CM-Sephadex C-25 cation-exchange resin was added to bind the protein and the culture was incubated in an incubator shaker at 4° C. and 110 rpm for 2 days. The slurry was poured through Miracloth® into a Buchner funnel and the resin was collected and packed into a FPLC (Fast protein liquid chromatography) column. The resin was extensively washed with a binding buffer and the bound protein was then eluted in 1 M NaCl, 50 mM Tris, pH 7.6. FPLC fractions were collected and concentrated at 4° C. in an Amicon Stirred Cell concentrator (Cole-Parmer, Vernon Hills, IL) with a membrane that had a molecular weight cut-off (MWCO) of 3,000. The concentrated fraction was dialyzed in tubing against 10 mM Tris, pH 7.6. The dialysate was used for HPLC analysis with retention time 32 min. HPLC elute was lyophilized. The lyophilized protein was re-suspended in nuclease-free water and the protein concentration was determined by using Nanodrop (NanoDrop™ 2000/2000c Spectrophotometers). The high-performance liquid chromatography of the Pichia pastoris-expressed NCR169 resulted in only one peak. The purified peptide at the concentration of 12 μM was found not to have any antifungal activity against Botrytis cinerea.

What is claimed is:

1. A composition comprising a nodule-specific cysteine rich NCR peptide folding variant 1 (NCR_PFV1),
wherein said NCR peptide is a cationic 6-Cys NCR peptide comprising in contiguous amino acid sequence from its N-terminal end (1) a first cysteine pair motif including C₁ and C₂, (2) a first intervening sequence (IS1), (3) a second cysteine pair motif including C₃ and C₄, (4) a second intervening sequence (IS2), and (5) a third cysteine pair motif including C₅ and C₆, wherein said NCR peptide has less than 60% sequence identity to CaNCR7 (SEQ ID NO: 2), CaNCR13 (SEQ ID NO: 1), CaNCR14 (SEQ ID NO: 3), or CaNCR15 (SEQ ID NO: 4),
wherein said cysteine residues C₁, C₂, C₃, C₄, C₅, and C₆ of NCR_PFV1 form a first set of disulfide bonds and NCR_PFV1 thereby adopts a first conformation that exhibits greater antimicrobial activity as compared to a nodule-specific cysteine rich NCR peptide folding variant 2 (NCR_PFV2) that has the same amino acid sequence as NCR_PFV1 but forms a distinct second set of disulfide bonds between cysteine residues C₁, C₂, C₃, C₄, C₅, and C₆ and adopts a distinct second conformation, and
wherein said composition is substantially free of NCR_PFV2.

2. The composition of claim 1, wherein said first set of disulfide bonds comprises a C₁-C₄ disulfide bond, a C₂-C₅ disulfide bond, and a C₃-C₆ disulfide bond.

3. The composition of claim 2, wherein said second set of disulfide bonds comprises disulfide bonds other than a C₁-C₄ disulfide bond and a C₂-C₅ disulfide bond.

4. The composition of claim 3, wherein said second set of disulfide bonds comprises a C₁-C₂ disulfide bond, a C₃-C₆ disulfide bond, and a C₄-C₅ disulfide bond.

5. The composition of any one of claims 1 to 4, wherein said cationic 6-Cys NCR has an isoelectric point between 8.0 and 12.0.

6. The composition of any one of claims 1 to 4, wherein said NCR_PFV1 exhibits greater protease resistance than said NCR_PFV2.

7. The composition of any one of claims 1 to 4, wherein said first cysteine pair motif is a C₁-5AA-C₂ motif that comprises in contiguous sequence from its N-terminal end five amino acids (A₁, A₂, A₃, A₄, and A₅) flanked by an N-terminal C₁ and a C-terminal C₂.

8. The composition of any one of claims 1 to 4, wherein said A₅ is an anionic amino acid selected from the group consisting of Asp (D) and Glu (E) or a cationic amino acid selected from the group consisting of His (H), Arg (R), and Lys (K) or an aliphatic amino acid selected from the group consisting of Gly (G), Ala (A), Val (V), Leu (L), Ile (I), and Pro (P).

9. The composition of any one of claims 1 to 4, wherein said A₄ is a cationic amino acid selected from the group consisting of His (H), Arg (R), and Lys (K) or an aliphatic amino acid selected from the group consisting of Gly (G), Ala (A), Val (V), Leu (L), Ile (I), and Pro (P) or an amide amino acid selected from the group consisting of Asn (N) and Gln (Q) or a polar amino acid selected from the group consisting of Ser (S) and Thr (T).

10. The composition of any one of claims 1 to 4, wherein said A₃ is a cationic amino acid selected from the group consisting of His (H), Arg (R), and Lys (K) or an anionic amino acid selected from the group consisting of Asp (D) and Glu (E) or a polar amino acid selected from the group consisting of Ser (S) and Thr (T).

11. The composition of any one of claims 1 to 4, wherein said A₅ is an anionic amino acid selected from the group consisting of Asp (D) and Glu (E), wherein said A₄ is a cationic amino acid selected from the group consisting of His (H), Arg (R), and Lys (K) and wherein said A₃ is an anionic amino acid selected from the group consisting of Asp (D) and Glu (E).

12. The composition of any one of claims 1 to 4, wherein said second cysteine pair motif is a C₃-5-10AA-C₄ motif that comprises in contiguous sequence from its N-terminal end from five to ten amino acids (A₆, A₇, A₈, A₉, A₁₀ up through A₁₁, A₁₂, A₁₃, A₁₄, A₁₅) flanked by an N-terminal C₃ and a C-terminal C₄.

13. The composition of any one of claims 1 to 4, wherein said second cysteine pair motif is a C₃-5-10AA-C₄ motif that comprises in contiguous sequence from its N-terminal end seven amino acids (A₆, A₇, A₈, A₉, A₁₀, A₁₁, and A₁₂) flanked by an N-terminal C₃ and a C-terminal C₄.

14. The composition of any one of claims 1 to 4, wherein said A₆ is an aliphatic amino acid selected from the group consisting of Gly (G), Ala (A), Val (V), Leu (L), Ile (I), and Pro (P) or a cationic amino acid selected from the group consisting of His (H), Arg (R), and Lys (K).

15. The composition of any one of claims 1 to 4, wherein said A₁₂ is an anionic amino acid selected from the group consisting of Asp (D) and Glu (E) or a cationic amino acid selected from the group consisting of His (H), Arg (R), and Lys (K) or an amide amino acid selected from the group consisting of Asn (N) and Gln (Q).

16. The composition of any one of claims 1 to 4, wherein said A₆ and said A₁₂ are cationic amino acids selected from the group consisting of His (H), Arg (R), and Lys (K).

17. The composition of any one of claims 1 to 4, wherein said third cysteine pair motif is a C₅-1AA-C₆ motif that comprises one amino acid (A₁₆) flanked by an N-terminal C₅ and a C-terminal C₆.

18. The composition of any one of claims 1 to 4, wherein said A₁₆ is a cationic amino acid selected from the group consisting of His (H), Arg (R), and Lys (K) or an aliphatic amino acid selected from the group consisting of Gly (G), Ala (A), Val (V), Leu (L), Ile (I), and Pro (P) or an anionic amino acid selected from the group consisting of Asp (D) and Glu (E).

19. The composition of any one of claims 1 to 4, wherein said A₁₆ is a cationic amino acid selected from the group consisting of His (H), Arg (R), and Lys (K).

20. The composition of any one of claims 1 to 4, wherein said IS1 comprises from one to eight amino acids or from four to five amino acids.

21. The composition of any one of claims 1 to 4, wherein said IS2 comprises from four to six amino acids.

22. The composition of any one of claims 1 to 4, wherein said IS1 and said IS2 each consists of four amino acids.

23. The composition of any one of claims 1 to 4, wherein said NCR_PFV1 comprises an amino acid sequence having at least 60%, 70%, 75%, 80%, 85%, 90%, 95%, 97%, or 99% sequence identity to any one of SEQ ID NO:8 to 371.

24. The composition of any one of claims 1 to 4, wherein said composition further comprises an agriculturally, pharmaceutically, or veterinary practicable carrier, diluent, or excipient.

25. A composition comprising a nodule-specific cysteine rich NCR peptide folding variant 1 (NCR_PFV1),
wherein said NCR peptide is a cationic 4-Cys NCR comprising in contiguous amino acid sequence from its N-terminal end (1) a first cysteine pair motif including C₁ and C₂, (2) a first intervening sequence (IS1), and (3) a second cysteine pair motif including C₃ and C₄, and wherein said NCR peptide has less than 60% sequence identity to CaNCR7 (SEQ ID NO: 2), CaNCR13 (SEQ ID NO: 1), CaNCR14 (SEQ ID NO: 3), or CaNCR15 (SEQ ID NO: 4),
wherein said NCR_PFV1 cysteine residues C₁, C₂, C₃, and C₄ form a first set of disulfide bonds and NCR_PFV1 thereby adopts a first conformation that exhibits greater antimicrobial activity as compared to a nodule-specific cysteine rich NCR peptide folding variant 2 (NCR_PFV2) that has the same amino acid sequence as NCR_PFV1 but forms a distinct second set of disulfide bonds between cysteine residues C₁, C₂, C₃, C₄, C₅, and C₆ and adopts a distinct second conformation, and
wherein said composition is substantially free of NCR_PFV2.

26. The composition of claim 25, wherein said first set of disulfide bonds comprises a C₁-C₂ disulfide bond and a C₃-C₄ disulfide bond.

27. The composition of claim 26, wherein said second set of disulfide bonds comprises disulfide bonds other than a C₁-C₂ disulfide bond and a C₃-C₄ disulfide bond.

28. The composition of claim 27, wherein said second set of disulfide bonds comprises a C₁-C₃ disulfide bond and a C₂-C₄ disulfide bond.

29. The composition of claim 25, wherein said first set of disulfide bonds comprises a C₁-C₃ disulfide bond and a C₂-C₄ disulfide bond.

30. The composition of claim 29, wherein said second set of disulfide bonds comprises disulfide bonds other than a C₁-C₃ disulfide bond and a C₂-C₄ disulfide bond.

31. The composition of claim 25, wherein said first set of disulfide bonds comprises a C₁-C₄ disulfide bond and a C₂-C₃ disulfide bond.

32. The composition of claim 31, wherein said second set of disulfide bonds comprises disulfide bonds other than a C₁-C₄ disulfide bond and a C₂-C₃ disulfide bond.

33. The composition of any one of claims 25 to 32, wherein said cationic 4-Cys NCR has an isoelectric point of from 8.0 to 12.0.

34. The composition of any one of claims 25 to 32, wherein said NCR_PFV1 exhibits greater protease resistance as compared to said NCR_PFV2.

35. The composition of any one of claims 25 to 32, wherein said first cysteine pair motif is a C₁-5AA-C₂ motif that comprises in contiguous sequence from its N-terminal end five amino acids (A₁, A₂, A₃, A₄, and A₅) flanked by an N-terminal C₁ and a C-terminal C₂.

36. The composition of any one of claims 25 to 32, wherein said second cysteine pair motif is a C₃-4AA-C₄ motif that comprises in contiguous sequence from its N-terminal end four amino acids (A₆, A₇, A₈, and A₉) flanked by an N-terminal C₃ and a C-terminal C₄, and

37. The composition of any one of claims 25 to 32, wherein said IS1 comprises from five to thirteen amino acids or from five to eleven amino acids.

38. The composition of any one of claims 25 to 32, wherein said A₅ is an anionic amino acid selected from the group consisting of Asp (D) and Glu (E).

39. The composition of any one of claims 25 to 32, wherein said A₄ is a cationic amino acid selected from the group consisting of His (H), Arg (R), and Lys (K).

40. The composition of any one of claims 25 to 32, wherein said A₃ is an anionic amino acid selected from the group consisting of Asp (D) and Glu (E).

41. The composition of any one of claims 25 to 32, wherein said A₆ is a cationic amino acid selected from the group consisting of His (H), Arg (R), and Lys (K) or an aliphatic amino acid selected from the group consisting of Gly (G), Ala (A), Val (V), Leu (L), Ile (I), and Pro (P).

42. The composition of any one of claims 25 to 32, wherein said A₇ is a cationic amino acid selected from the group consisting of His (H), Arg (R), and Lys (K) or an amide amino acid selected from the group consisting of Asn (N) and Gln (Q) or an aliphatic amino acid selected from the group consisting of Gly (G), Ala (A), Val (V), Leu (L), Ile (I), and Pro (P).

43. The composition of any one of claims 25 to 32, wherein said A₈ is an aliphatic amino acid selected from the group consisting of Gly (G), Ala (A), Val (V), Leu (L), Ile (I), and Pro (P) or an amide amino acid selected from the group consisting of Asn (N) and Gln (Q) or a cationic amino acid selected from the group consisting of His (H), Arg (R), and Lys (K).

44. The composition of any one of claims 25 to 32, wherein said NCR_PFV1 comprises an amino acid sequence having at least 60%, 70%, 75%, 80%, 85%, 90%, 95%, 97%, or 99% sequence identity to any one of SEQ ID NO: 8 to 371, optionally wherein said NCR_PFV1 does not comprise the amino acid sequence of NCR169 (SEQ ID NO: 6) or NCR247 (SEQ ID NO: 5).

45. The composition of any one of claims 25 to 32, wherein the composition further comprises an agriculturally, pharmaceutically, or veterinary practicable carrier, diluent, or excipient.

46. A method for making a composition of any one of claims 1-4 or 25-32, said method comprising:
(a) separating a fraction comprising NCR peptide folding variant 1 (NCR_PFV1) from one or more fractions comprising NCR peptide folding variant 2 (NCR_PFV2), or NCR_PFV2 peptide fragments thereof, from a mixture comprising NCR_PFV1 and NCR_PFV2, or NCR_PFV2 peptide fragments thereof, and
(b) combining said fraction comprising NCR_PFV1, or a preparation further purified from said NCR_PFV1 fraction, with an agriculturally, pharmaceutically, or veterinary practicable carrier, diluent, or excipient, thereby making the composition.

47. The method of claim 46, further comprising obtaining said mixture comprising NCR_PFV1 and NCR_PFV2, or NCR_PFV2 peptide fragments thereof, by
(a) culturing in fermentation broth microorganisms expressing a recombinant polynucleotide comprising a transcriptional promoter operably linked to a polynucleotide encoding a signal peptide that is in frame with and upstream of a polynucleotide encoding NCR_PFV1, or a variant thereof, and
(b) separating the microorganisms from the fermentation broth, which comprises the mixture,
wherein said microorganisms are optionally selected from the group consisting of Candida, Kluveromyces, Hansenula, Pichia, Saccharomyces, Schizosaccharomyces, or Yarrowia, Myceliophthora, and Escherichia.

48. The method of claim 46, further comprising obtaining said mixture comprising NCR_PFV1 and NCR_PFV2, or NCR_PFV2 peptide fragments thereof, by
(a) culturing in fermentation broth microorganisms expressing a recombinant polynucleotide comprising a transcriptional promoter operably linked to a polynucleotide encoding NCR_PFV1, or a variant thereof, and
(b) separating the microorganisms, which comprises the mixture, from the fermentation broth,
(c) disrupting the microorganisms to obtain a cell lysate, and
(d) separating insoluble debris in the cell lysate from an aqueous fraction comprising said mixture,
wherein said microorganisms are optionally selected from the group consisting of Candida, Kluveromyces, Hansenula, Pichia, Saccharomyces, Schizosaccharomyces, or Yarrowia, Myceliophthora, and Escherichia.

49. The method of claim 47 or 48, further comprising treating said mixture comprising NCR_PFV1 and NCR_PFV2 with a protease under non-denaturing conditions that are sufficient to produce NCR_PFV2 peptide fragments but insufficient to produce NCR_PFV1 peptide fragments.

50. The method of claim 49, wherein the protease is a trypsin family serine protease that cleaves NCR_PFV2 at the carboxy terminus of an NCR_PFV2 Arg (R) or Lys (K).

51. The method of claim 50, wherein the trypsin family serine protease is a recombinant bovine, porcine, human, or microbial trypsin, and, optionally, wherein the microbial trypsin is a Streptomycete trypsin or variant thereof.

52. The method of claim 49, further comprising separating the NCR_PFV1 from the NCR_PFV2 peptide fragments by size-exclusion chromatography.

53. A method for preventing or reducing crop damage or post-harvest loss by a plant pathogenic microbe, said method comprising contacting a plant, a plant seed, pre- or post-harvest grain, pre- or post-harvest fruit, or pre- or post-harvest vegetable with an effective amount of the composition of any one of claims 1-4 or 25-32 and under conditions suitable for preventing or reducing crop damage or post-harvest loss.

54. The method of claim 53, wherein the plant pathogenic microbe is selected from the group consisting of Fusarium sp., Alternaria sp., Verticillium sp., Phytophthora sp., Colletotrichum sp., Botrytis sp., Cercospora sp., Phakopsora sp., Rhizoctonia sp., Sclerotinia sp., Pythium sp., Phoma sp., Leptosphaeria sp., Gaeumannomyces sp., Puccinia sp., Septoria sp., Penicillium sp., Lasiodiplodia sp., Phomopsis sp., Mycosphaerella sp., Golovinomyces sp., Erisyphe sp., Albugo sp., Setosphaeria sp., Cochiobolus sp., Helminthosporium sp., Diplodia sp. or Stenocarpella sp.

55. The method of claim 53, wherein the plant, plant seed, pre- or post-harvest grain, pre- or post-harvest fruit, or pre- or post-harvest vegetable is selected from the group consisting of a cereal crop, a legume crop, a root or tuber crop, an oil crop, a fruit crop, a vegetable crop, a nut crop, a forage or turf grass crop, a forage legume, a drug crop, a spice or flavoring crop, a fiber crop, and a biofuel crop plant, a plant seed, pre- or post-harvest grain, pre- or post-harvest fruit, or pre- or post-harvest vegetable.

56. The method of claim 53, wherein the plant, a plant seed, pre- or post-harvest grain, pre- or post-harvest fruit, or pre- or post-harvest vegetable is selected from the group consisting of wheat, rice, corn, barley, oat, sorghum, rye, millet, peanut, chickpea, pigeon pea, lentil, kidney bean, soybean, lima bean, potato, sweet potato, cassava, canola, peanut, palm, coconut, safflower, cotton, sunflower, flax, olive, sugarcane, sugarbeet, banana, orange, apple, pear, breadfruit, pineapple, strawberry, grape, cherry, coffee, coco, tomato, lettuce, carrot, melon, asparagus, Brassica sp., cashew, walnut, pistachio, almond, alfalfa, clover, castor, Camelina, pennycress, switchgrass, Miscanthus, and Jatropha plant, plant seed, pre- or post-harvest grain, pre- or post-harvest fruit, or pre- or post-harvest vegetable.

57. A medical device having antimicrobial properties, said medical device comprising a medical device in operable combination with a composition of any one of claims 1-4 or 25-32, wherein said medical device comprises at least one surface that is topically coated or impregnated with said composition.

58. The medical device of claim 57, wherein said medical device is selected from the group consisting of a stent, a catheter, a contact lens, a condom, a patch, and a diaphragm.

59. A method for treating, preventing, or inhibiting a microbial infection in a subject in need thereof, said method comprising administering to said subject a therapeutically effective amount of a composition of any one of claims 1-4 or 25-32 under conditions and for a duration suitable for treating, preventing, or inhibiting said microbial infection.

60. The method of claim 59, wherein said administering comprises topical, enteral, parenteral, or intravenous introduction of the composition to the subject.

61. The method of claim 59, wherein the subject is selected from the group consisting of human, livestock, poultry, fish, and companion animal.

62. The method of claim 59, wherein the microbial infection is of a mucosal membrane, eye, skin, or a nail and the composition is applied to the mucosal membrane, eye, skin, or nail.

63. The method of claim 59, wherein the microbial infection is by a dermatophyte, and wherein the dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

64. The method of claim 59, wherein the microbial infection is by a microorganism selected from the group consisting of Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, and Blastomyces species.

65. The method of claim 64, wherein the microbial infection is by a microorganism selected from the group consisting of Candida species, wherein the Candida species is selected from the group consisting of Candida albicans (C. albicans), C. auris, C. glabrata, C. parasilosis, C. tropicalis, and C. krusei.

66. The composition of any one of claims 1-4 or 25-32 for use in a method for treating, preventing, or inhibiting microbial infection in a subject in need thereof.

67. The composition of claim 66, wherein the subject is selected from the group consisting of human, livestock, poultry, fish, and companion animals.

68. A plant part that is at least partly coated with the composition of any one of claims 1-4 or 25-32.

69. The plant part of claim 68, wherein said plant part is selected from the group consisting of a seed and a pre- or post-harvest grain.

70. The plant part of claim 68, wherein said plant part is selected from the group consisting of pre- or post-harvest fruit, pre- or post-harvest vegetable, and pre- or post-harvest flower.

71. The plant part of claim 68, wherein the plant part is selected from the group consisting of cereal crop, legume crop, root or tuber crop, oil crop, fruit crop, vegetable crop, nut crop, forage or turf grass crop, forage legume, drug crop, spice or flavoring crop, fiber crop, and biofuel crop plant part.

72. The plant part of claim 68, wherein the plant part is selected from the group consisting of wheat, rice, corn, barley, oat, sorghum, rye, millet, peanut, chickpea, pigeon pea, lentil, kidney bean, soybean, lima bean, potato, sweet potato, cassava, canola, peanut, palm, coconut, safflower, cotton, sunflower, flax, olive, sugarcane, sugarbeet, banana, orange, apple, pear, breadfruit, pineapple, strawberry, grape, cherry, coffee, coco, tomato, lettuce, carrot, melon, asparagus, Brassica sp., cashew, walnut, pistachio, almond, alfalfa, clover, castor, Camelina, pennycress, switchgrass, Miscanthus, and Jatropha plant part.
